(19) United States
(12) Patent Application Publication
(10) Pub. No.: US 2022/0378896 A1
(43) Pub. Date: Dec. 1, 2022

(54) VACCINE COMPOSITIONS AND METHODS FOR REDUCING TRANSMISSION OF STREPTOCOCCUS PNEUMONIAE

(71) Applicant: St. Jude Children's Research Hospital, Memphis, TN (US)

(72) Inventors: Jason W. Rosch, Memphis, TN (US); Hannah M. Rowe, Memphis, TN (US)

(73) Assignee: St. Jude Children's Research Hospital, Memphis, TN (US)

(21) Appl. No.: 17/602,414

(22) PCT Filed: Mar. 12, 2020
(86) PCT No.: PCT/IB2020/052250

§ 371 Date: Oct. 8, 2021

Related U.S. Application Data
(60) Provisional application No. 62/817,748, filed on Mar. 13, 2019.

(51) Int. Cl. A61K 39/09; A61P 31/04; C12Q 1/689; C12N 15/10

(52) U.S. Cl. CPC A61K 39/092; A61P 31/04; C12Q 1/689; C12N 15/1058; A61K 2039/55588

(57) ABSTRACT

Compositions and methods are provided for reducing the mammalian transmission of Streptococcus pneumoniae (S. pneumoniae) through the administration to mammalian subjects of vaccine compositions comprising at least one immunogenic polypeptide comprising a S. pneumoniae protein or a fragment or variant thereof that is required for or involved in transmission of the bacteria between mammalian hosts. These vaccine compositions also serve to reduce the incidence rate of at least one invasive disease caused by S. pneumoniae. Methods are also provided for identifying additional genetic factors involved in mammalian transmission of S. pneumoniae.

### FIELD OF THE INVENTION

The invention relates to the field of immunology and bacteriology. In particular, the invention relates to vaccines for reducing the transmission of Streptococcus pneumoniae (S. pneumoniae). The methods and compositions can be used to treat pneumonia and other invasive diseases associated with S. pneumonia infection.

### REFERENCE TO A SEQUENCE LISTING SUBMITTED ELECTRONICALLY AS A TEXT FILE

The instant application contains a Sequence Listing which has been submitted in ASCII format via EFS-Web and is hereby incorporated by reference in its entirety. Said ASCII copy, created on Mar. 12, 2020, is named S884351220WO_SeqList_ST25_3-12-20.txt, and is 653 KB in size.

### BACKGROUND OF THE INVENTION

Introduction of the pneumococcal conjugate vaccine has greatly reduced the burden of invasive disease by Streptococcus pneumoniae (S. pneumoniae), however rates of colonization and pneumonia (McLaughlin et al. (2018) Clin Infect Dis., published online May 24, 2018, DOI: 10.1093/cid/ciy312) remain largely equivalent due to serotype replacement (Azarian et al. (2018) PLoS Pathog. 14(4), e1006966; published online Apr. 5, 2018, DOI: 10.1371/journal.ppat.1006966) and limited efficacy of the vaccine at the mucosal surface. Critical to the success of S. pneumoniae is the capacity of the organism to initially colonize the human nasopharynx and subsequently transmit and colonize a new host. As such, both colonization and transmission dynamics reflect strong evolutionary pressures on this pathogen within populations and are key for understanding epidemiology. Despite the acknowledgement that transmission is a fundamental aspect of pneumococcal biology, there remains limited understanding of the bacterial and host factors that underlie this process when compared to our understanding of invasive disease.

Streptococcus pneumoniae (the pneumococcus) is a member of the human nasal microbiome, especially of children (van den Bergh (2012) PLoS One. 7(10), e47711, published online Oct. 20, 2012, DOI: 10.1371/journal.pone.0047711). Colonization can progress to invasive diseases such as otitis media, pneumonia, sepsis and meningitis. Pneumococcal transmission can be inferred from studies of human populations, by monitoring nasal colonization dynamics of children (Azarian et al. (2018)). Seasonal patterns of pneumococcal disease and colonization patterns support a role of respiratory viruses in promoting pneumococcal transmission, particularly the Influenza A virus (Althouse et al. (2017) Epidemiol Infect 145:2750-2758; Grijalva et al. (2014) Clin Infect Dis 58:1369-1376). An infant mouse model of pneumococcal transmission has been developed (Kono et al. (2016) PLoS Pathog 12, e1005887; Zafar et al. (2016) Infect Immun. 84(9) 2714-2722; Zafar et al. (2017a) MBio 8, e00989-17; Zafar et al. (2017b) Cell Host Microbe 21:73-83; Zangari et al. (2017) MBio. 8(2), published online Mar. 16, 2017, DOI: 10.1128/mBio.00188-17) and has shown valuable insights into the importance of capsule type (Zafar et al. (2017a)) and the contribution of pneumolysin (Zafar et al. (2017b)) for transmission but are not ideal for large scale genetic screens, as only a single bacteria is transmitted from donor to contact pup (Kono et al. (2016)). The present invention provides a model that is able to overcome these population bottlenecks to provide insight into factors required for mammalian transmission of S. pneumoniae.

### SUMMARY OF THE INVENTION

Compositions and methods are provided for reducing the mammalian transmission of Streptococcus pneumoniae (S. pneumoniae) through the administration to mammalian subjects of vaccine compositions comprising at least one immunogenic polypeptide comprising a S. pneumoniae protein or a fragment thereof that is required for or involved in transmission between mammalian hosts. Additional methods for reducing the mammalian transmissibility of S. pneumoniae comprise increasing levels or activity of proteins that decrease tolerance of desiccation stress or reducing levels or activity of proteins required for or involved in successful transmission. Thus, methods are provided for reducing the incidence rate of at least one invasive disease caused by S. pneumoniae, such as acute otitis media, pneumonia, sepsis, bacteremia, and meningitis.

Methods are also provided for identifying genetic factors involved in mammalian transmission of S. pneumoniae, wherein the methods comprise infecting an influenza co-infected ferret with a ferret-transmissible strain of S. pneumoniae comprising a gene mutant library, and analyzing members of the gene mutant library that are able to colonize, but exhibit a reduced transmission rate to contact ferrets.

### BRIEF DESCRIPTION OF THE FIGURES

FIG. 1 depicts pneumococcal transmission in ferrets. FIG. 1A shows the bacterial burden in ketamine-induced sneezes from donor and contact ferrets by days post-infection of donor animals. Each dot represents a single animal. In some iterations of the experiment, animals were sneezed daily, and in others, they were sneezed every other day. FIG. 1B shows donor-to-contact bottlenecks: percentage of inserts recovered from donor also recovered from cagemate contact. FIG. 1C shows the percent donor-to-contact bottleneck depending on the number of inserts in donor. FIG. 1D shows the percent donor-to-contact bottleneck depending on the bacterial burden (CFU) in donor.

FIG. 2 shows the fitness landscape of S. pneumoniae genes during mammalian transmission. Transposon insertions displaying reduced transmission in the ferret model as determined by absence in recipient animals while present in 5 or greater of 9 donor animals indicating not a colonization defect or enhanced transmission (pyruvate metabolism genes) as determined by recovery from all 21 recipient animals without being over-represented in donor animals. Gene designations for both BHN97 and TIGR4 are indicated. Predicted gene functionality is indicated by functional grouping. Genes with a homolog in either TIGR4 or D39 model strains are indicated by a solid border and genes without homology in either TIGR4 or D39 are indicated by a dotted border.

FIG. 3 shows that metabolic factors for transmission enhance environmental stability. FIG. 3A shows the confirmation of hyper-transmissibility of a targeted deletion of hyper transmitter spxB (SP_0730) in infant mice. Infant mice, when 4 days old, one half of each litter was infected intranasally with 2000 CFU of wild type or mutant pneumococcus. Pups were sampled daily for colonization by taping the nares on an agar plate with days until contact pups are colonized defined as two consecutive positive samples. Each panel represents data from between at least 10-20 contact pups were sampled per strain, from at least four independent litters. FIG. 3B shows the complementation of SpxB deletion on transmissibility. FIG. 3C provides the bacterial burden in nasal passages of donor pups 10 days post challenge. FIG. 3D shows the percent bacterial survival following desiccation and 24 hours incubation at room temperature compared to CFU/mL prior to desiccation. As deletion of SpxB in a serotype 4 strain eliminates capsule expression, a strain where a secondary mutation restores capsule expression was also examined for the TIGR4 strain (SpxB capsule+). FIG. 3E provides percent bacterial survival of SpxB complemented strain following desiccation and 24 hours incubation at room temperature compared to CFU/mL prior to desiccation. FIG. 3F shows the percent bacterial survival 24 hours post desiccation following two hour growth in carbohydrate free CY media. FIG. 3G provides the percent bacterial survival 24 hours post desiccation following two hour growth in metal depleted media. Statistics calculated by Mantel-Cox log-rank for transmission and for percent survival comparisons, Mann-Whitney test was used. A P-value <0.05 considered significant (*) with the respective P-values indicated for each panel.

FIG. 4 shows the confirmation in infant mice of genes required for mammalian transmission predicted by the ferret screen. C57/B16 mice were mated and one half of each litter of pups when 4 days old were infected intranasally with 2000 CFU wild type or mutant or complemented pneumococcal strain. FIG. 4A depicts the results for the targeted cppA (SP_1449) deletion. FIG. 4B depicts the results for the targeted comD (SP_2236) deletion. FIG. 4C depicts the results for the targeted piaA (SP_1032) deletion. Pups were sampled daily for colonization by taping the nares on an agar plate colonization of contact pups defined as two consecutive positive samples. Each panel represents data from between 10-20 contact pups that were sampled per strain, from at least 4 independent litters. FIG. 4D provides the bacterial burden in nasal passages of donors 10 days post challenge. For transmission, statistics were calculated by Mantel-Cox log-rank test and bacterial burden compared by Mann-Whitney using Prism 6. A P-value <0.05 was considered significant (*) with the respective P-values indicated for each comparison indicated in the respective panels, n.s.=non-significant.

FIG. 5 shows the purification of recombinant CppA (A) and recombinant PiaA (B). Western blots are provided with sera from immunized mice against cell fractions (C, D, E) and purified proteins (F, G, H) using sera from alum vaccinated (C, F), rCppA vaccinated (D, G), and rPiaA vaccinated (E, H) animals. ELISA results are provided against whole cell lysates (PCV-13) (I) or purified proteins (J), reported as log titer, inverse of last serum dilution with reading above background.

FIG. 6 shows that maternal vaccination with transmission factors blocks pneumococcal transmission in non-vaccinated offspring. Dams were vaccinated 2 weeks prior to mating, and every subsequent two weeks with recombinant protein vaccines based on transmission factors PiaA and CppA or 1:50 human dose PCV-13, and alum controls. One half of each litter was infected intranasally with 2000 CFU pneumococcal strain BHN97. FIG. 5A depicts bacterial burden in donor nasal passages 10 days post infection. FIGS. 5B-5E depict days until contact pups become colonized (two consecutive positive samples). FIG. 6B shows dam vaccinated with rPiaA, FIG. 6C shows dam vaccinated with rCppA, FIG. 6D shows dam vaccinated with both rCppA and rPiaA, and FIG. 6E shows dam vaccinated with PCV-13. FIG. 6F shows cross fostering, days until contact pups become colonized, placental=dam vaccinated with rCppA, foster dam unvaccinated. Lactation=dam unvaccinated, foster dam vaccinated with rCppA. For transmission, statistics were calculated by Mantel-Cox log-rank test and bacterial burden compared by Mann-Whitney using Prism 6. A P-value <0.05 was considered significant (*) with the respective P-values indicated for each comparison indicated in the respective panels, n.s.=non-significant.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### 1. Overview

Described herein is the first genetic screen for those factors involved in transmission of S. pneumoniae between mammalian hosts. Targeting one or more of these transmission factors with vaccines can be used to block pathogen spread in populations and reduce or eliminate invasive disease caused by S. pneumoniae in whole populations. Compositions and methods are provided herein for reducing the mammalian transmission of S. pneumoniae and/or reducing the incidence rate of at least one invasive disease caused by S. pneumonia (such as acute otitis media, pneumonia, sepsis, bacteremia, and meningitis) by administering to a mammalian subject infected with S. pneumoniae or at risk of infection by S. pneumoniae a vaccine composition comprising at least one immunogenic polypeptide comprising a S. pneumoniae protein (or an immunogenic fragment or variant thereof) that is required for or involved in transmission between mammalian hosts. Alternatively, methods are provided for reducing the levels or activity of one or more proteins required for or involved in transmission in order to reduce the mammalian transmissibility of S. pneumoniae and/or reducing the incidence rate of at least one invasive disease caused by S. pneumonia.

As described herein, when the expression of particular S. pneumoniae genes is reduced, the fitness of S. pneumoniae is enhanced and the bacteria are less prone to desiccation stress. Thus, additional methods for reducing the mammalian transmissibility of S. pneumoniae and/or reducing the incidence rate of at least one invasive disease caused by S. pneumoniae comprise increasing the levels or activity of proteins that decrease tolerance of desiccation stress.

Methods are also provided for identifying additional genetic factors involved in mammalian transmission of S. pneumoniae, wherein the methods comprise infecting an influenza co-infected ferret with a ferret-transmissible strain of S. pneumoniae comprising a gene mutant library, and analyzing members of the gene mutant library that are able to colonize, but exhibit a reduced transmission rate to contact ferrets.

2. Factors Involved in Mammalian Transmission of S. pneumoniae

A genetic screen in a ferret model of S. pneumoniae infection and transmission uncovered factors required for or involved in mammalian transmission of the bacteria. Specifically, Table 1 provides S. pneumoniae proteins (SEQ ID NOs: 1-87) that when genes encoding these proteins are disrupted or deleted, these bacteria were unable to transmit to recipient animals. Thus, these genes and encoded proteins are required for mammalian transmission of S. pneumoniae. An additional 118 S. pneumoniae proteins are provided in Table 2 (SEQ ID NOs: 88-205) that when genes encoding these proteins are disrupted or deleted, these bacteria exhibited a significantly reduced transmission rate to recipient animals. These genes and encoded proteins are required for normal or optimal levels of transmission of S. pneumoniae.

TABLE 1

| | | | Mean | Number | | | Identified | | |
|---|---|---|---|---|---|---|---|---|---|
| SEQ | BHN97 | TIGR4 | Probability | of | | | in Murine | | |
| ID | Locus | Locus | of | Donors | Functional | | Nasopharynx | | |
| NO: | ID | ID | Infection | Infected | Classification | Name | TnSeq* | KEGG prediction** | BHN97 protein sequence |
| 1 | peg.0181 | n/a | 0 | 8 | translation | | | Is required not only | MSIFIGGAWPYANGSLHIGHAAALLPGDILARYYR |
| | | | | | | | | for elongation of | QKGEEVLYVSGSDCNGTPISIRAKKENKSVKEIADF |
| | | | | | | | | protein synthesis but | YHKEFKETFEKLGFTYDLYSRTDSPLHHEIVQELFL |
| | | | | | | | | also for the initiation | QLYEKKFLYTKKIKQLYCTFDNQFLPDRFVEGKCP |
| | | | | | | | | of all mRNA | NCGTHSRGDQCDNCSAILDPIDLVDKRCSICSNEPE |
| | | | | | | | | translation through | VRETEHFYYVFSEFQNLLETYLNDAEETVRWRKN |
| | | | | | | | | initiator tRNA(fMet) | AINLTKRYLREGLPDRAVTRDLPNGIPVPIDGFRDK |
| | | | | | | | | aminoacylation (By | KIYVWFEAVAGYYTASVDWAQKLQNNITDFWNN |
| | | | | | | | | similarity) | RTKSYYVHGKDNIPFHTIIWPAILSGLEIEPLPEYIIS |
| | | | | | | | | | SEYLTLENKKISTSNNWAIWLNDIIKKYDADSIRYF |
| | | | | | | | | | LTINAPEMKDANFSWREFIYSHNSELLGSYGNFINR |
| | | | | | | | | | TLKFIEKYFESEIPTKYLEGEILYNLKELYTTVGNLV |
| | | | | | | | | | ESGHMKQALEEIFEYIRSANKFYDDMKPWALRESD |
| | | | | | | | | | IEKCKEVLATCVIIILNLGQMLNPFIPFSGKKIEDMF |
| | | | | | | | | | KTKLNTWNYISNLPNKLSDVSMLFDRIDLKKIDEE |
| | | | | | | | | | VLELQQTSSR |
| 2 | peg.1585 | SP_0252 | 0 | 8 | CHO | | | Transaldolase is | MEFMLDTLNLDEIKKWSEILPLAGVTSNPTIAKREG |
| | | | | | metabolism | | | important for the | SINFFERIKDVRELIGSTPSIHVQVISQDFEGILKDAH |
| | | | | | | | | balance of | EIRRQAGDDIFIKVPVTPAGLRAIKVLKKEGYHITA |
| | | | | | | | | metabolites in the | TAIYTVIQGLLAIEAGADYLAPYYNRMENLNIDSNS |
| | | | | | | | | pentose-phosphate | VIRQLALAIDRQNSPSKILAASFKNVAQVNNALAA |
| | | | | | | | | pathway (By | GAHAVTAGADVFESAFAMPSIQKAVDDFSDDWFV |
| | | | | | | | | similarity) | TQNSRSI |
| 3 | peg.0031 | SP0913 | 0 | 7 | bacteriocin | | | ABC transporter, | MFRLTNKLAVSNLIKNRKLYYPFALAVLLAVTVTY |
| | | | | | | | | permease | LFYSLTFNPKIAEIRGGTTIQATLGFGMFVVTLASAI |
| | | | | | | | | | IVLYANSFVMKNRSKELGIYGMLGLEKRHLISMTF |
| | | | | | | | | | KELVVFGILTVGAGIGIGALFDKLIFAFLLKLMKLK |
| | | | | | | | | | VELVATFQMNVVIAVLVVFGLIFLGLMFLNALRIA |
| | | | | | | | | | RMNALQLSREKASGEKRGRFLPLQTILGSISLGIGY |
| | | | | | | | | | YLALTVTDPLTALTTFFLAVLLVIFGTYLLFNAGIT |
| | | | | | | | | | VFLQILKKNKKYYYQPNNLISVSNLIFRMKKNAVG |
| | | | | | | | | | LATIAILSTMVLVTMSAATSIFNSAESFKKVLNPHD |
| | | | | | | | | | FGVSVQNVEKEDLDKLLSQFASDKGYSVKEKEVL |
| | | | | | | | | | RYSNFGIANQEGTKLTIFEKGQNRVQPTTVFMVFD |
| | | | | | | | | | QKDYENMTGQKLSLSGNEVGLFAKNDGLKGQKA |
| | | | | | | | | | LTLNDHQFSVKEEFNKDFIVNHVPNKFNILTTDYN |
| | | | | | | | | | YLVVPDLQAFLDQFPDSAIYNQFYGGMNVNVSEE |
| | | | | | | | | | EQLKVAEEYENYLNQFNAQLDTEGSYVYGSNLAD |
| | | | | | | | | | ASSQMSALFGGVFFIGIFLSIIFMVGTVLVIYYKQIS |
| | | | | | | | | | EGYEDRERFIILQKVGLDQKQIKQTINKQVLTVFFL |
| | | | | | | | | | PLLFAFIHLAFAYHMLSLILKVIGVLDTTMMLIVTL |
| | | | | | | | | | SICAIFLIAYVLIFMITSRSYRKIVQM |
| 4 | peg.0182 | n/a | 0 | 7 | signaling | | | protein tyrosine | LNNLTLLKEYNFRDLGNHLTQTGQKIKPKTLFRSS |
| | | | | | | | | serine phosphatase | KLFGISKIDVDLLQSYGITKVIDFRSANEIKKAPDPD |
| | | | | | | | | | IKNIKNIVIPIFYNDDSELTEFPIEFFNKSDAGFQHMI |
| | | | | | | | | | KTYDQMINQKQSKLGYKKFFKLLLSHPKDESLLFH |
| | | | | | | | | | CSMGKDRTGIASLFLLYILGVDMNDIFHDYLLSNK |
| | | | | | | | | | YLINVRKENIEYVNNHSGNVILMHNLLSLSSAKEE |
| | | | | | | | | | YINRVLNVLDKEYGGILRYINTELGISSQEIEELKDR |
| | | | | | | | | | YLF |
| 5 | peg.0843 | n/a | 0 | 7 | phage | | | NA | MAKAITDATFEQETKDGLVLVDFWATWCGPCRM |
| | | | | | | | | | QGPILDKLSEELSEDVLKIVKMDVDENPNTARAFGI |
| | | | | | | | | | MSIPTLLFKKDGQVVKQVAGVHTAEQIKAIIAELS |
| 6 | peg.1235 | SP2146 | 0 | 7 | CHO | | | Alpha-L-fucosidase | MKKIKPHGPLPSQTQLAYLGDELAAFIHFGPNTFY |
| | | | | | metabolism | | | | DQEWGTGQEDPERFNPSQLDAREWVRVLKETGFK |
| | | | | | | | | | KLILVVKHHDGFVLYPTAHTDYSVKVSPWRRGKG |
| | | | | | | | | | DLLLEVSQAATEFDMDMGVYLSPWDAHSPLYHV |
| | | | | | | | | | DREADYNAYYLAQLKEILSNPNYGNAGKFAEVW |
| | | | | | | | | | MDGARGEGAQKVNYEFEKWFETIRDLQGACLIFST |
| | | | | | | | | | EGTSIRWIGNERGYAGDPLWQKVNPDKLGTEAEL |
| | | | | | | | | | NYLQHGDPSGTIFSIGEADVSIRPGWFYHEDQDPKS |
| | | | | | | | | | LEELVEIYFHSVGRGTPLLLNIPPNQAGLFDAKDIE |
| | | | | | | | | | RLYEFATYRNELYKEDLALGAEVSGPALSADFACR |
| | | | | | | | | | HLTDGLETSSWANDAALPIQLELDLGSPKTFDVIEL |
| | | | | | | | | | REDLKLGQRIAAFHVQVEVDGVWQEFGSGHTVGY |
| | | | | | | | | | KRLLRGAVVEAQKIRVVITESQVLPLLTKISLYKTP |
| | | | | | | | | | RLSQTVAVQGLAFAEKSLAVAKGETLHFRIERSESS |
| | | | | | | | | | SSLEAKISIQPGTGVHGVAYQDEIYVLAFQAGETEK |
| | | | | | | | | | SLTLPTLYFAGDKTLDFYLNLTVDGQLVDQLQVQ |
| | | | | | | | | | VS |
| 7 | peg.1239 | SP2152 | 0 | 7 | transport | | | c4-dicarboxylate | MSEKAKKGFKMPSSYTVLLIIIAIMAVLTWFIPAGA |
| | | | | | | | | anaerobic carrier | FIEGIYETQPQNPQGIWDVLMAPIRAMLGTHPEEGS |
| | | | | | | | | | LIKETSAAIDVAFFILMVGGFLGIVNKTGALDVGIA |
| | | | | | | | | | SIVKKYKGREKMLILVLMPLFALGGTTYGMGEET |
| | | | | | | | | | MAFYPLLVPVMMAVGFDSLTGVAIILLGSQIGCLA |
| | | | | | | | | | STLNPFATGIASATAGVGTGDGIVLRLIFWVTLTAL |
| | | | | | | | | | STWFVYRYADKIQKDPTKSLVYSTRKEDLKHFNV |
| | | | | | | | | | EESSSVESTLSSKQKSVLFLFVLTFILMVLSFIPWTD |
| | | | | | | | | | LGVTIFDDFNAWLTGLPVIGNIVGSSTSALGTWYFP |
| | | | | | | | | | EGAMLFAFMGILIGVIYGLKEDKIISSFMNGAADLL |
| | | | | | | | | | SVALIVAIARGIQVIMNDGMITDTILNWGKEGLSGL |
| | | | | | | | | | SSQVFIVVTYIFYLPMSFLIPSSSGLASATMGIMAPL |
| | | | | | | | | | GEFVNVRPSLIITAYQSASGVLNLIAPTSGIVMGAL |
| | | | | | | | | | ALGRINIGTWWKFMGKLVVAIIVVTIALLLLGTFLP |
| | | | | | | | | | FL |
| 8 | peg.1635 | n/a | 0 | 7 | regulation | | | | MYHIKADKRSLASARLISQGLNRLLQAKDFDSITV |
| | | | | | | | | | TDIVNEAGVGRATFYRLFDNVTDVITYSYYIIAHPT |
| | | | | | | | | | LEKIQNKFISNILDFSKSLINDFYSQDSILLISTLLFSV |
| | | | | | | | | | QNGYHFLSMLNLYKNDLLVLLKNEKNLKTKSQDY |
| | | | | | | | | | KYLMEIYLMTLMVTVGSYIQKPEITRELLLEKVSN |
| | | | | | | | | | VWQDLTHLHINN |
| 9 | peg.1750 | SP0397 | 0 | 7 | CHO | | | mannitol-1-phosphate | MKHSVHFGAGNIGRGFIGEILFKNGFHIDFVDVNN |
| | | | | | metabolism | | | 5-dehydrogenase | QIIHALNEKGKYEIEIAQKGQSRIEVTNVAGINSKEH |
| | | | | | | | | | PEQVIEAIQKTDIITTAIGPNILPFIAELLAKGIEARRV |
| | | | | | | | | | AGNTQVLDVMACENMIGGSQFLYQEVKKYLSPEG |
| | | | | | | | | | LTFADNYIGFPNAAVDRIVPAQSHEDSLFVVVEPFN |
| | | | | | | | | | EWVVETKRLKNPDLRLKDVHYEEDLEPFIERKLFS |
| | | | | | | | | | VNSGHATSAYIGAHYGAKTILEALQNPNIKSRIESV |
| | | | | | | | | | LAEIRSLLIAKWNFDKKELENYHKVIIERFENPFIVD |
| | | | | | | | | | EVSRVARTPIRKLGYNERFIRPIRELKELSLSYKNLL |
| | | | | | | | | | KTVGYAFDYRDVNDEESIRLGELLAKQSVKDVVIQ |
| | | | | | | | | | VTGLDDQELIEQIVEYI |
| 10 | peg.0148 | SP1032 | 0 | 6 | Metal | PiaA | | Periplasmic binding | MKNKFFLIAILAMCIVFSACSSNSVKNEENTSKEHA |
| | | | | | transport | | | protein | PDKIVLDHAFGQTILDKKPERVATIAWGNHDVALA |
| | | | | | | | | | LGIVPVGFSKANYGVSADKGVLPWTEEKIKELNGK |
| | | | | | | | | | ANLFDDLDGLNFEAISNSKPDVILAGYSGITKEDYD |
| | | | | | | | | | TLSKIAPVAAYKSKPWQTLWRDMIKIDSKALGME |
| | | | | | | | | | KEGDELIKNTEARISKELEKHPEIKGKIKGKKVLFT |
| | | | | | | | | | MINAADTSKFWIYTSKDPRANYLTDLGLVFPESLK |
| | | | | | | | | | EFESEDSFAKEISAEEANKINDADVIITYGDDKTLE |
| | | | | | | | | | ALQKDPLLGKINAIKNGAVAVIPDNTPLAASCTPTP |
| | | | | | | | | | LSINYTIEEYLNLLGNACKNAK |
| 11 | peg.0152 | SP1035 | 0 | 6 | Metal | PiaD | | ABC transporter | MKGLWSNNLTCGYDEKIILENINIKIPEEKISVIIGSN |
| | | | | | transport | | | | GCGKSTLIKTLSRLIKPLEGEVLLDNKSINSYKEKD |
| | | | | | | | | | LAKHIAILPQSPIIPESITVADLVSRGRFPYRKPFKSL |
| | | | | | | | | | GKDDLEIINRSMVKANVEDLANNLVEELSGGQRQ |
| | | | | | | | | | RVWIALALAQDTSILLLDEPTTYLDISYQIELLDLLT |
| | | | | | | | | | DLNQKYKTTICMILHDINLTARYADYLFAIKEGKL |
| | | | | | | | | | VAEGKPEDILNDKLVKDIFNLEAKIIRDPISNSPLMI |
| | | | | | | | | | PIGKHHVNS |
| 12 | peg.0159 | SP1040 | 0 | 6 | recombination | | | Resolvase | MKAFAIYNDYEIVGEYEDAGKSGKSIEGRIQFNRM |
| | | | | | | | | | MEDIKSGKDGVSFVLVFKLSRFARNAADVLSTLQI |
| | | | | | | | | | MQDYGVNLICVEDGIDSSKDAGKLMISVLSAVAEI |
| | | | | | | | | | ERENIRIQTMEGCIQKAREGKWNGGFAPYGYKLED |
| | | | | | | | | | GKLFINEEEAVAIRTIFDQYVNTTIGANGISKYLENH |
| | | | | | | | | | GIRKIPRQNGKNPLFDAGLIRKILKNPVYHGKIAFG |
| | | | | | | | | | RRTLEKVHGTRNEYKQVEQDEYLISEGIHEAIVSDE |
| | | | | | | | | | VWQAAQVKLKSQAKKYEHVNKGKDTRTHLLSGI |
| | | | | | | | | | VKCPICGVGMFGNKCIKKKKDGTKYKDFYYYGCK |
| | | | | | | | | | HRQMIRGHKCTFSKQIREELLDDAVAEVIVKIVSNP |
| | | | | | | | | | KFASMMQEKINMKVDTSEIEKEIDNYQKELRKSHS |
| | | | | | | | | | TKFKLIEEIDNLDVEDKHYKRRKQDLDDRLYRMY |
| | | | | | | | | | DKIDELESSLIDAKAKKQTIEAEKLTGDNIYKVLIYF |
| | | | | | | | | | DKLYKVMNDVERRQLISALISEIQVYEEKQSNGQW |
| | | | | | | | | | LKSITFKLPIIEENLNIDLDNDEQVECVSLLEKRS |
| 13 | peg. 0186 | SP0638 | 0 | 6 | transport | | Yes | ABC transporter, | MKKYQRMHLIFIRQYLKQIMEYKADFLVGVVGVF |
| | | | | | | | | permease | LTQGLNMLFLNILFQHIPLLDGWSFHQVAFIYGFSL |
| | | | | | | | | | IPKGIDHLFFDNLWALGQHLIRKGEFDKYLTRPISP |
| | | | | | | | | | LFHILVETFQIDALGELLVGVLLLLMTITSLTWTWA |
| | | | | | | | | | KVFLFLISIPFATLIYTSLKIVTASIAFWTKQSGAIIYI |
| | | | | | | | | | FYMFNDFAKYPIAIYHSFLRWLISFIIPFAFTAYYPA |
| | | | | | | | | | SYFLKDKDGLFNIGGLILISLIFFTLSLKLWNKGLDA |
| | | | | | | | | | YESAGS |
| 14 | peg.0256 | SP1125 | 0 | 6 | CHO | | Yes | Glycerate kinase | MKIVIAPDSFKESLTAQQVAEAIKRGFQQSIADVEY |
| | | | | | metabolism | | | | LLCPVGDGGEGTVDAIRHSLDLKEKWIQVTDPFGQ |
| | | | | | | | | | KEAMCYFQKGELALFEVADLVGLGKIPLEKRNPLQ |
| | | | | | | | | | IQTCGIGELILHLISKGIKDIYIGVGSTASNDGGLGIA |
| | | | | | | | | | AGLGYQFYDRDGNVLPASSQSLLNLASVSTENCY |
| | | | | | | | | | KIPEGVQIHILADVVSPLCGHQGATYTFGNQKGLH |
| | | | | | | | | | PTMFAVVDQAIQDFYEKFSPATLEIKGAGAGGGLA |
| | | | | | | | | | GGLCAFAQASIVSGIDTLLGLNQL |
| 15 | peg.0384 | SP_1275 | 0 | 6 | CHO | | | carbamoyl-phosphate | MPKRTDIQKIMVIGSGPIIIGQAAEFDYAGTQACLS |
| | | | | | metabolism | | | synthetase ammonia | LKEEGYEVVLVNSNPATIMTDKEIADKVYIEPITLE |
| | | | | | | | | chain | FVTRILRKEGPDALLPTLGGQTGLNMAMELSKNGI |
| | | | | | | | | | LDELGVELLGTKLSAIDQAEDRDLFKQLMEELEQPI |
| | | | | | | | | | PESEIVNTVEEAVAFAATIGYPVIVRPAFTLGGTGG |
| | | | | | | | | | GMCANEKELREITENGLKLSPVTQCLIERSIAGFKEI |
| | | | | | | | | | EYEVMRDSADNALVVCNMENFDPVGIHTGDSIVF |
| | | | | | | | | | APAQTMSDYENQMLRDASLSIIRALKIEGGCNVQL |
| | | | | | | | | | ALDPNSFKYYVIEVNPRVSRSSALASKATGYPIAKL |
| | | | | | | | | | AAKIAVGLTLDEVINPVTGSTYAMFEPALDYVVAK |
| | | | | | | | | | IPRFPFDKFEKGERRLGTQMKATGEVMAIGRNIEES |
| | | | | | | | | | LLKACRSLEIGVHHNEIPELAAVSDDTLIEKVVKAQ |
| | | | | | | | | | DDRLFYVSEAIRRGYTPEEIAELTKIDIFYLDKLLHI |
| | | | | | | | | | FEIEQELGAHPQDLEVLKTAKLNGFSDRKIAELWG |
| | | | | | | | | | TTDDKVRQLRLENKIVPVYKMVDTCAAEFDSETP |
| | | | | | | | | | YFYSTYGWENESIKSDKESVLVLGSGPIRIGQGVEF |
| | | | | | | | | | DYATVHSVKAIQAAGYEAIIMNSNPETVSTDFSVS |
| | | | | | | | | | DKLYFEPLTFEDVMNVIDLEQPKGVIVQFGGQTAI |
| | | | | | | | | | NLAEPLAKAGVTILGTQVADLDRAEDRDLFEQALK |
| | | | | | | | | | ELDIPQPPGQTATNEEEAALAARKIGFPVLVRPSYV |
| | | | | | | | | | LGGRAMEIVENEEDLRSYMRTAVKASPDHPVLVD |
| | | | | | | | | | SYIVGQECEVDAISDGKNVLIPGIMEHIERAGVHSG |
| | | | | | | | | | DSMAVYPPQTLSQKVQETIADYTKRLAIGLHCLGM |
| | | | | | | | | | MNIQFVIKDEKVYVIEVNPRASRTVPFLSKVTNIPM |
| | | | | | | | | | AQVATKLILGQSLSELGYQNGLYPESTRVHIKAPVF |
| | | | | | | | | | SFTKLAKVDSLLGPEMKSTGEVMGSDATLEKALY |
| | | | | | | | | | KAFEASYLHLPTFGNVVFTIADDAKEEALNLARRF |
| | | | | | | | | | QNIGYGILATEGTAAFFASHGLQAQPVGKIGDDDK |
| | | | | | | | | | DIPSFVRKGRIQAIINTVGTKRTADEDGEQIRRSAIE |
| | | | | | | | | | HGVPLFTALDTANAMLKVLESRSFVTEAI |
| 16 | peg.0386 | SP_1277 | 0 | 6 | Amino acid | | Yes | aspartate | MSENQQALNHVVSMEDLTVDQVMKLIKRGIEFKN |
| | | | | | metabolism | | | transcarbamylase | GAQLPYEDHPIVSNLFFEDSTRTHKSFEVAEIKLGL |
| | | | | | | | | | ERLDFDVKTSSVNKGETLYDTILTLSALGVDVCVIR |
| | | | | | | | | | HPEVDYYRELIASPTITTSIINGGDGSGQHPSQSLLD |
| | | | | | | | | | LMTIYEEFGHFEGLKVAIAGDLDHSRVAKSNMQIL |
| | | | | | | | | | KRLGSELFFAGPEEWRSQEFADYGQFVTIDEIIDQV |
| | | | | | | | | | DVMMFLRVQHERHDSGAVFSKEDYHAQHGLTQE |
| | | | | | | | | | RYDRLKETAILMHPAPINRDVEIADHLVEAPKSRIV |
| | | | | | | | | | QQMTNGVFVRMAILESVLASRNAN |
| 17 | peg.0483 | SP1391 | 0 | 6 | Protein | | Yes | Putative membrane | MTIHIIITMLLLLAFLIGSIWFAKKKYQINLAVLGLG |
| | | | | | processing | | | peptidase family | AVAFFVSSQILEKLVHILILHPQKDGSIALLQDHPLI |
| | | | | | | | | (DUF2324) | YIIYGLAMAAFFEETARLVFFKWLEKKRSLEKADA |
| | | | | | | | | | LAYGLGHGGLELIFLGLTSLLNLYIVLSAVQTQNPQ |
| | | | | | | | | | VMQLLSENMLKTIQSLSVWQIYLLGFERILALGFQL |
| | | | | | | | | | LLTVWVYQAVRQKKWIYLLAAYGLHAFFDLAPSL |
| | | | | | | | | | FQVGWLTNPVLVEVILALELVLVAYGTKEIFCKKS |
| 18 | peg.0730 | SP1654 | 0 | 6 | CHO | | Yes | Alpha-L-fucosidase | MIRNKKQDYVLAYKQPASTTYMSWEEEALPIGNG |
| | | | | | metabolism | | | | SLGAKVFGLIGAERIQFNEKSLWSGGPLPDSSDYQ |
| | | | | | | | | | GGNLQDQYVFLAEIRQALEKRDYNLAKELAEQHLI |
| | | | | | | | | | GPKTSQYGTYLSFGDIHIEFSQQGTTLSQVTDYQRQ |
| | | | | | | | | | LNISKALVTTSYVYKGTRFEREAFASFPDDLLVQRF |
| | | | | | | | | | TKEGAETLDFTIELSLTCDLASDEKYEQEKSDYKEC |
| | | | | | | | | | KLDITD SHILMKGRVKDNDLRFASYLAWETDGDIR |
| | | | | | | | | | VWSDRVQISGASYANLFLAAKTDFAQNPASNYRK |
| | | | | | | | | | KLDLEQQVIDLVDTAKEKGYTQLKSRHIEDYQALF |
| | | | | | | | | | QRVQLDLEADVDASTTDDLLKNYKPQEGQALEEL |
| | | | | | | | | | FFQYGRYLLISSSRDCPDALPANLQGVWNAVDNPP |
| | | | | | | | | | WNSDYHLNVNLQMNYWPAYVTNLLEAVFPVINY |
| | | | | | | | | | VDDLRVYGRLAAVKYAGIVSQKGEENGWLVHTQ |
| | | | | | | | | | ATPFGWTAPGWDYYWGWSPAANAWMMQTVYE |
| | | | | | | | | | AYSFYRDQDYLREKIYPMLRETVRFWNAFLHKDQ |
| | | | | | | | | | QAQRWVSSPSYSPEHGPISIGNTYDQSLIWQLFHDF |
| | | | | | | | | | IQAAQELGLDEDLLTEVKEKSDLLNPLQITQSGRIR |
| | | | | | | | | | EWYEEEEQYFQNEKVEAQHRHASHLVGLYSGNLF |
| | | | | | | | | | SYKGQEYIEAARASLNDRGDGGTGWSKANKINLW |
| | | | | | | | | | ARLGDGNRAHKLLAEQLKTSTLPNLWCSHPPFQID |
| | | | | | | | | | GNFGATSGMAEMLLQSHAAYLVPLAALPDAWST |
| | | | | | | | | | GSVSGLMARGHFEVSMSWEDKKLLQLTILSRSGG |
| | | | | | | | | | DLRVSYPDIEKSVIKMNQEKIKAKCMGKDCISVAT |
| | | | | | | | | | AEGDLVQFYF |
| 19 | peg.0904 | SP1840 | 0 | 6 | transport | | | ABC transporter | MQNKQEQWAVLKRLMSYLKPYGLLTFLALSFLLA |
| | | | | | | | | | TTVIKSVIPLVASHFIDQYLSNLNQLAVTVLLVYYG |
| | | | | | | | | | LYILQTVVQYVGNLLFARVSYSIVRDIRRDAFANM |
| | | | | | | | | | EKLGMSYFDKTPAGSIVSRLTNDTETISDMFSGILSS |
| | | | | | | | | | FISAVFIFLTTLYTMLVLDFRLTALVLLFLPLIFLLV |
| | | | | | | | | | NLYRKKSVKIIEKTRSLLSDINSKLAENIEGIRIIQAF |
| | | | | | | | | | NQEKRLQAEFDEINQEHLVYANRSVALDALFLRPA |
| | | | | | | | | | MSLLKLLGYAVLMAYFGYRGFSIGITVGTMYAFIQ |
| | | | | | | | | | YINRLFDPLIEVTQNFSTLQTAMVSAGRVFALIDER |
| | | | | | | | | | TYEPLQENGQAKVQEGNIRFEHVCFSYDGKHPILD |
| | | | | | | | | | DISFSVNKGETIAFVGHTGSGKSSIINVLMRFYEFQS |
| | | | | | | | | | GRVLLDDVDIRDFSQEELRKNIGLVLQEPFLYHGTI |
| | | | | | | | | | KSNIAMYQEISDDQVQAAAAFVDADSFIQELPQGY |
| | | | | | | | | | DSPVSERGSSFSTGQRQLLAFARTVASQPKILILDE |
| | | | | | | | | | ATANIDSETESLVQASLAKMRQGRTTIAIAHRLSTI |
| | | | | | | | | | QDANCIYVLDKGRIIESGTHEELLTLGGTYHKMYS |
| | | | | | | | | | LQAGAMAYTL |
| 20 | peg.1016 | SP1962 | 0 | 6 | unknown | | | NA | MTPEEMYLTERLDVQIAHFLKKSVQHRRRYKVLKI |
| | | | | | | | | | TEIVAGFLIAVFCAIPMPGDRYRLISVALSSLGLLCE |
| | | | | | | | | | GIINLYNAKENWISYQKTAQLLEKEKFLYQCQTEK |
| | | | | | | | | | YAGKTKAFALFVKTCEGLISEEINQWESIQSKEVAA |
| | | | | | | | | | SADAPVKKE |
| 21 | peg.1044 | SP1956 | 0 | 6 | bacteriocin | | | bacteriocin- | MSQKLNHLDVGEFVLLLPEHLRSEEEHYKSVFEDD |
| | | | | | | | | associated integral | LTSRMSSQDERQQMTATVGYLESGQDRFVYNTTPI |
| | | | | | | | | membrane | SYQQFLKDPIIIVITPQSTGPQSILFWIDAVQNYVLF |
| | | | | | | | | | NQLSDAQELIQRQGIENWVSEMQTGYHNYITLLDN |
| | | | | | | | | | IQRERWVMLAGAVLGIATSILLFNTMNRLYFEEFR |
| | | | | | | | | | RAIFIKRIAGLRFLEIHRTYLFAQLGVFLLGFVASVF |
| | | | | | | | | | LQVEVGVAFLVLLLFTGLSLLQLHVQMQKENKMS |
| | | | | | | | | | MLVLKGG |
| 22 | peg.1101 | SP2013 | 0 | 6 | transport | | | abc transporter | MGKKRWARNGSESNDASYAQVVSLYEDTSISVSN |
| | | | | | | | | permease protein | NETDKVLAGSLYTDTNEQGLTIPSSLLKNWNEQTG |
| | | | | | | | | | KNLTANDLIGKSVSASIVESAAEASKIAQFQTKIVR |
| | | | | | | | | | VINDEDDMEDSNSFMLSYQMETILKEAGFTKAVSY |
| | | | | | | | | | FILELKDPSQTKVVTEELQKNKKYTVLSQQRVLDI |
| | | | | | | | | | VITFIRVIQGLLIVLSSQAIVVAAVMIGIIIYINIMQRS |
| | | | | | | | | | KEIGVMKAVGYQNRDVKGIFIYEAIWIVGIALLLAF |
| | | | | | | | | | LVAQGVGSLANAIVSHFYPSITKVFELNLLSVLGTL |
| | | | | | | | | | VFALLLGYVSAYFPARKISKMDPVESLRYE |
| 23 | peg.1190 | SP2103 | 0 | 6 | translation | | | Methyltransferase | MNTNLKPKLQRFASATAFACPICQENLTLLETNFK |
| | | | | | | | | | CCNRHSFDLAKFGYVNLAPQIKQSANYNKENFQN |
| | | | | | | | | | RQQILEAGFYQAILDAVSDLLASSKTTTTILDIGCGE |
| | | | | | | | | | GFYSRKLQESHSEKTFYAFDISKDSVQIAAKSEPNW |
| | | | | | | | | | AVNWFVGDLARLPIKDANMDILLDIFSPANYGEFR |
| | | | | | | | | | RVLSKDGILIKVIPTENHLKEIRQRVQDQLTNKEYS |
| | | | | | | | | | NQDIKEHFQEHFTILSSQTASLTKTITAEQLQALLS |
| | | | | | | | | | MTPLLFHVDQSKIDWSQLTEITIEAEILVGKAF |
| 24 | peg.1234 | SP2145 | 0 | 6 | CHO | | | Alpha-1,2- | MKPLLETIDTRFGTASKHAFSRGNTLPYTGVPFGM |
| | | | | | metabolism | | | mannosidase | NYFVPQTSDQDGSCFFDPHLPIFQGIRLTHQPSPWI |
| | | | | | | | | | GDYSWLLLTPVTSQLGGDSLFHRQSSYDRDKASFQ |
| | | | | | | | | | PHYLKIFSLRYQIETQLTPTCYGASIRLEQKQGKTLS |
| | | | | | | | | | LYLHVADELTVEQVDKRTLALRQEGKTETNKNSL |
| | | | | | | | | | TMFTALQMNTDILAISQEAGDWRIDLASSQTEMQL |
| | | | | | | | | | ATSFISPSQALINLPQEDFDSCKASAQADWENLLHR |
| | | | | | | | | | FDIIETGEADRTFFDHCLYRLFLFPQTFYEVDESGQ |
| | | | | | | | | | AIHMDLATGTVKPGVLFSNNGFWDTFRTTFPLFAL |
| | | | | | | | | | IIPEHYQRFLEGFLNSYRDTGFLPKWLAPDERGMM |
| | | | | | | | | | PGTLLDGIIADSACKDMAPDLEGELFQAMLKTASK |
| | | | | | | | | | ADPLGINGRHGLAQYQELGYLSTDHHESVSHTLDY |
| | | | | | | | | | AYSDFCIANCAKKLKNIEIAETYKAASQNYRQLFD |
| | | | | | | | | | AETGYMRARDNQGNFHPDFSPYSWGRDYAECSAI |
| | | | | | | | | | QATLGVLHDIPGLIQLMGGKETFSNYLLKACQDAP |
| | | | | | | | | | LFETTGYGYEIHEMSEMATAPFGQIAISNQPSFHIPY |
| | | | | | | | | | LFRYSDYPDYTALLIKTLRQKAFHPSWEAYPGDED |
| | | | | | | | | | NGSLSAWYIWSALGFYPTCPGKPSYDLGIPLFDHL |
| | | | | | | | | | RVYLAKENKWLDIHTEQNHSHFNFVKECRLDKTL |
| | | | | | | | | | VSTIQHQDLLKAEQLTFTLSWLPNH |
| 25 | peg.1334 | SP2236 | 0 | 6 | competence | ComD | Yes | Histidine kinase | MDFFLLVDLILYFLIISHSYRLICKDQINRKELFFFG |
| | | | | | | | | | AYTLLTEIVLDFPFYLLYLDGLGIATFLFPLGLYSYF |
| | | | | | | | | | RWMKQYERDRGLFLSLLLSLLYESTHNFLSVTFSSI |
| | | | | | | | | | TGDNFVLQYYGLFFFVVTVLTYFVTLKIIYYFHLEL |
| | | | | | | | | | AYFDKDYLYPFLKKVFFALLLLHIVSFVSDMVSTIK |
| | | | | | | | | | HLNSFGSILSSIVFISLLLTFFAMNSHKVQMEKEIAL |
| | | | | | | | | | KQKKFEQKHLQNYTDEIVGLYNEIRGFRHDYAGM |
| | | | | | | | | | LVSMQMAIDSGNLQEIDRVYNEVLVKANHKLRSD |
| | | | | | | | | | KYTYFDLNNIEDSALRSLVAQSIVYARNNGVEFTL |
| | | | | | | | | | EVKDTITKLPIELLDLVRIMSVLLNNAVEGSADSYK |
| | | | | | | | | | KQMEVAVIKMETETVIVIQNSCKMTMTPSGDLFAL |
| | | | | | | | | | GFSTKGRNRGVGLNNVKELLDKYNNIILETEMEGS |
| | | | | | | | | | TFRQIIRFKRKFE |
| 26 | peg.1455 | n/a | 0 | 6 | Unknown | | | | MKNKRYFFDTILIILLLISTIFCVSPVFIKLDILGTPSH |
| | | | | | | | | | AILTFVLAIPLFYILSQCLHTLLLLVSSIFCKLRPIYF |
| | | | | | | | | | YFIFVIIIGARKYYRILFHQLMGFSPGIAVFYKESQT |
| | | | | | | | | | TKNLFKFYYFLYFTTLISYYFFFTFVYDKPLLLPLIS |
| | | | | | | | | | FSIIIALVQKLYRIENQQLFLLKSKVLTILESKKNCEF |
| | | | | | | | | | NLQDYHEIWKLQSKSELPCVALSYISLIKPYLSESV |
| | | | | | | | | | REQIDLLEVKRFKKINHPISLYGMLDVIKLNLYLRH |
| | | | | | | | | | YNEKNKYESMLKKILEVRPDFVLIEQNIDDSLNSSQ |
| | | | | | | | | | PLSLSLAISEIQLLLEVYMGIKHVSIRR |
| 27 | peg.1463 | SP0117 | 0 | 6 | unknown | | | Choline binding | MNKKKMILTSLASVAILGAGFVTSQPTFVRAEEAP |
| | | | | | | | | protein A | VASQSKAEKDYDAAMKKSEAAKKAYEEAKKKAE |
| | | | | | | | | | DAQKKYDEGQKKTEEKARKAEEASKEIAKATSEV |
| | | | | | | | | | QNAYVKYQRVQRNSRLNEKERKKQLAEIDEEINK |
| | | | | | | | | | AKQILNEKNEDFKKVREEVIPEPTELAKDQRKAEE |
| | | | | | | | | | AKAEEKVAKRKYDYATLKVALAKSKVEAEEAELD |
| | | | | | | | | | NKAENLQNKVADLEKEIANAEKTVADLEKEVAKL |
| | | | | | | | | | EKDVEDFKNSNGEQAEQYLAAAEKDLVAKKAELA |
| | | | | | | | | | EAKIKAATKKAELEKAEAELENLLSTLDPEGKTQD |
| | | | | | | | | | ELDKEAAEAELNKKVEALQNQVAELEEELSKLED |
| | | | | | | | | | NLKDAETNNVEDYIKEGLEEAIATKKAELEKTQKE |
| | | | | | | | | | LDAALNELGPDGDEEETPAPAPQPEKPAPAPAPKP |
| | | | | | | | | | EQPAPAPKPEQPAPAPKPEKSADQQAEEDYARRSE |
| | | | | | | | | | EEYNRLTQQQPPKAEKPAPAPAPKPEQPAPAPKTG |
| | | | | | | | | | WKQENGMWYFYNTDGSMATGWLQNNGSWYYL |
| | | | | | | | | | NSNGAMATGWLQYNGSWYYLNANGAMATGWA |
| | | | | | | | | | KVNGSWYYLNANGSMATGWVKDGDTWYYLEAS |
| | | | | | | | | | GAMKASQWFKVSDKWYYVNSNGAMATGWLQY |
| | | | | | | | | | NGSWYYLNANGAMATGWAKVNGSWYYLNANGS |
| | | | | | | | | | MATGWVKDGDTWYYLEASGAMKASQWFKVSDK |
| | | | | | | | | | WYYVNGSGSLAVNTTVDGYTVNENGEWV |
| 28 | peg.1817 | n/a | 0 | 6 | unknown | | | NA | MDKMKPVFQALNKELIQENLTLTIICVGGYVLEYH |
| | | | | | | | | | GLRATQDVDAFYDQNQKINEIIARVGKQFNLNTHE |
| | | | | | | | | | ELWLNNHVANMNKQPPLSLCESLYSFENLTVLVV |
| | | | | | | | | | PIEYVLGMKMMSIREQDLKDIGAIIKYKNFHSPFDT |
| | | | | | | | | | FKYLKDMGFDTIDLSVLLEGFSYAYGMDWLEKFF |
| | | | | | | | | | KENQDKLREFY |
| 29 | peg.1823 | SP0480 | 0 | 6 | transport | | | potassium transporter | MKIILVGGGKVGFALCRSLVAEKHDVLLIEQDEAV |
| | | | | | | | | peripheral membrane | LNHIVSRFDIIGILGNGADFAILEQASVQDCDIFIALT |
| | | | | | | | | | EHDEVNMIAAVLAKKMGAKETIVRVRNPEYSNSY |
| | | | | | | | | | FKEKNILGFSLIVNPELLAARAIANIIDFPNALSVERF |
| | | | | | | | | | AGGRVSLMEFVVKSTSGLCQMPISDFRKKFGNVIV |
| | | | | | | | | | CAIERDHQIIIPSGDMTVQDKDRIFVTGNRVDMILF |
| | | | | | | | | | HNYFKSRAVKSLLIVGAGRIAYYLLGILKDSRIDTK |
| | | | | | | | | | VIEINPEIASFFSEKFPNLYIVQGDGTAKDILLEESAQ |
| | | | | | | | | | HYDAVATLTGVDEENLITSMFLDRVGVQKNITKV |
| | | | | | | | | | NRTSLLEIINAPDFSSIITPKSIAVDTIMHFIRGRVNA |
| | | | | | | | | | QYSDLQAMHHLANGQIETLQFHIKEANKMTAKPL |
| | | | | | | | | | SQLKLKKGVLIAAIIRKGKTIFPTGEDMLEVGDKLL |
| | | | | | | | | | VTTLLPNITKIYDLIAR |
| 30 | peg.2077 | SP_0752 | 0 | 6 | Amino acid | | | ABC transporter | MALLEVKQLTKHFGGLTAVGDVTLELNEGELVGLI |
| | | | | | transport | | | | GPNGAGKTTLFNLLTGVYEPSEGTVTLDGHLLNGK |
| | | | | | | | | | SPYKIASLGLGRTFQNIRLFKDLTVLDNVLIAFGNH |
| | | | | | | | | | HKQHVFTSFLRLPAFYKSEKELKDKALELLKIFDLD |
| | | | | | | | | | GDAETLAKNLSYGQQRRLEIVRALATEPKILFLDEP |
| | | | | | | | | | AAGMNPQETAELTELIRRIKDEFKITIMLIEHDMNL |
| | | | | | | | | | VMEVTERIYVLEYGRLIAQGTPDEIKTNKRVIEAYL |
| | | | | | | | | | GGEA |
| 31 | peg.0039 | SP0921 | 0 | 5 | Polyamine | | | Agmatine deiminase | MMDSPKKLGYHMPAEYEPHHGTLMIWPTRPGSW |
| | | | | | metabolism | | | | PFQGKAAKRAFTQIIETIAEGERVYLLVEQAYLSEA |
| | | | | | | | | | QSYLGDKVVYLDIPTNDAWARDTGPTILVNDKGK |
| | | | | | | | | | KLAVDWAFNAWGGTYDGLYQDYEEDDQVASRFA |
| | | | | | | | | | EALERPVYDAKPFVLEGGAIHSDGQGTILVTESCLL |
| | | | | | | | | | SPGRNPNLTKEEIENTLLESLGAEKVIWLPYGIYQD |
| | | | | | | | | | ETNEHVDNVAAFVGPAELVLAWTDDENDPQYAM |
| | | | | | | | | | SKADLELLEQETDAKGCHFTIHKLPIPAVRQVVTEE |
| | | | | | | | | | DLPGYIYEEGEEKRYAGERLAASYVNFYIANKAVL |
| | | | | | | | | | VPQFEDVNDQVALDILSKCFPDRKVVGIPARDILLG |
| | | | | | | | | | GGNIHCITQQIPE |
| 32 | peg.0074 | SP0954 | 0 | 5 | competence | ComEC | | Competence protein | MLQWIKNFSIPLIYLSFLLLWLYYAIFSASYLALLGF |
| | | | | | | | | | VFLLVCLFIQFPWKSAGKVLIICGIFGFWFVFQNWQ |
| | | | | | | | | | QSQASQNLADSVERVRILPDTIKVNGDSLSFRGKSN |
| | | | | | | | | | GRAFQVYYKLQSEEEKEAFQALTDLHEIGLEGKLS |
| | | | | | | | | | EPEGQRNFGGFNYQAYLKTQGIYQTLNIKTIQSLQ |
| | | | | | | | | | KIGSWDIGENLSSLRRKAVVWIKTHFPDPMGNYM |
| | | | | | | | | | TGLLLGHLDTDFEEMNELYSSLGIIHLFALSGMQV |
| | | | | | | | | | GFFMNGFKKLLLRLGLTQEKLKWLTYPFSLIYAGL |
| | | | | | | | | | TGFSASVIRSLLQKLLAQHGVKGLDNFALTVLVLFI |
| | | | | | | | | | VMPNFFLTAGGVLSCAYAFILTMTSKEGEGLKAVT |
| | | | | | | | | | SESLVISLGILPILSFYFAEFQPWSILLTFVFSFLFDLV |
| | | | | | | | | | FLPLLSILFVLSFLYPVIQLNFIFEWLEGIIRLVSQVA |
| | | | | | | | | | RRPLVFGQPNAWLLILLLISLALVYDLRKNIKGLTV |
| | | | | | | | | | LSLLITGLFFLTKYPLENEITMLDVGQGESIFLRDVT |
| | | | | | | | | | GKTILIDVGGKAESYKKIKKWQEKMTTSNAQRTLI |
| | | | | | | | | | PYLKSRGVAKIDQLILTNTDKEHVGDLSEMTKAFH |
| | | | | | | | | | VGEILVSKDSLKQKEFVAELQATQTKVRSMIVGEN |
| | | | | | | | | | LPIFGSQLEVLSPRKMGDGGHDDTLVLYGKFLDKQ |
| | | | | | | | | | FLFTGNLEEKGEKDLLKHYPDLKVNVLKASQHGN |
| | | | | | | | | | KKSSSPAFLEKLKPELTLISVGKSNRMKLPHQETLT |
| | | | | | | | | | RLEGINSKVYRTDQQGAIRFKGLDSWKIESVR |
| 33 | peg. 0166 | SP1046 | 0 | 5 | CHO | | | alpha amylase, | MYYKQQISEVMFNLLDSHDTERILWTANEDVQLV |
| | | | | | metabolism | | | catalytic region | KSALAFFFLQKGTPCIYYGTELALTGGPDPDCRRC |
| | | | | | | | | | MPWERVSSDNDMLNFMKRLIKIRKYASVIISHGKY |
| | | | | | | | | | SLQEIKSDLVALEWKYEGRILKVIFNQSTEDYLLEK |
| | | | | | | | | | EAVALASNCQELENQLVISPDGFVIF |
| 34 | peg.0169 | SP1051 | 0 | 5 | plasmid | | | zeta toxin | MEIQDYTDSEFKHALARNLRSLTRGKKSSKQPIAIL |
| | | | | | | | | | LGGQSGAGKTTIHRIKQKEFQGNIVIIDGDSFRSQH |
| | | | | | | | | | PHYLELQQEYGKDSVEYTKDFAGKMVESLVTKLS |
| | | | | | | | | | SLRYNLLIEGTLRTVDVPKKTAQLLKNKGYEVQLA |
| | | | | | | | | | LIATKPELSYLSTLIRYEELYIINPNQARATPKEHHD |
| | | | | | | | | | FIVNHLVDNTRQLEELAIFERIQIYQRDRSCVYDSK |
| | | | | | | | | | ENTTSAADVLQELLFGEWSQVEKEMLQVGEKRLN |
| | | | | | | | | | ELLEK |
| 35 | peg.0185 | SP0637 | 0 | 5 | transport | | | transporter, permease | MTKLWKRYKPFVSAGIQELITYRVNFFLYRIGDVM |
| | | | | | | | | | GAFVAFYLWKAVFDSSHQSLIQGFTLSDMTLYIIM |
| | | | | | | | | | SFVTNLLTKSDSSFMIGWEVKDGSIIMRLLRPVHFA |
| | | | | | | | | | MSYLFTEIGSRWLVFVSVGLPFVILIAGLKLLSGESF |
| | | | | | | | | | LQIVLITTVYLLSLILAFLINFFSIFALVFQLLCLKTY |
| | | | | | | | | | GDQIF |
| 36 | peg.0248 | SP_1118 | 0 | 5 | CHO | | | pullulanase | MYNYPMRIHYHRKNGEYDTCSFVKSQDQRIDLLT |
| | | | | | metabolism | | | | YKEDYFGALFSFEHPSSHVIESLNFVVHTGQTSKEY |
| | | | | | | | | | SIRFNHYPLLTEVWILEGDDRIYYSENPAIASPFYKN |
| | | | | | | | | | QNPFAFDKAINSASFDHHWGYQGELGCRVEDNQA |
| | | | | | | | | | HFSLWSPTATEVQVVVYESAANDAPVWKTFEMKR |
| | | | | | | | | | GNSYSYNHKENTIGVWSLDVEENLVGKTYQYQVQ |
| | | | | | | | | | FPHHQTLTRDPYTIATSPDGKRSAILSHVEKQVENF |
| | | | | | | | | | EVKHGSEATWRLENPCKAVICEMHIRDLTKSPTSG |
| | | | | | | | | | VDEHLRGTFLGAAQAGTVNQYGQSTAFDYIKKLG |
| | | | | | | | | | YNYVQLQPIADRHKEYDEDGNVTYNWGYDPQNY |
| | | | | | | | | | NAPETSFSTNPDDPAQVIRDLKVMVQAYHDAGIGV |
| | | | | | | | | | IMDVVYNHTFSVVDAPFQTTVPDYYYRMNPDGTF |
| | | | | | | | | | QNGTGVGNETASEHEMFRKYMIDSLLYWVQEYNI |
| | | | | | | | | | DGFRFDLMGIHDVKTMQMIRQSLDEIDSNIILYGEG |
| | | | | | | | | | WDMGTGLAPYDKAKKDNAYQMPNIGFFNDNQRD |
| | | | | | | | | | AVKGGEVYGAIKSGFVSGAATEPILAKAILGSRELG |
| | | | | | | | | | SYTHPNQVLNYVEAHDNYNLHDLLATLHPDQSSE |
| | | | | | | | | | QIMRKVETATAMNLLMQGMAFMEIGQEFGRTKLV |
| | | | | | | | | | ATGENGELTHDDRERAMNSYNAPDSVNQVNWNLI |
| | | | | | | | | | NERQDSIEFIRQVIRLKTKTGAFSYSSYDEIYHHVFV |
| | | | | | | | | | HSAIEHSGCLIYEVHGKEHLLVVVNAKSEPYQFEN |
| | | | | | | | | | AGNLAMLVTNSRSKEDNVLNDISLAVLSVL |
| 37 | peg.0249 | SP_1119 | 0 | 5 | CHO | | | Aldehyde | LTRYQNLVNGKWKSSEQEITIYSPINQEELGTVPA |
| | | | | | metabolism | | | dehydrogenase | MTQTEADEAMQAARAALPAWRALSAVERAAYLH |
| | | | | | | | | | KTAAILERDKEEIGTILAKEVAKGIKAAIGEVVRTA |
| | | | | | | | | | DLIRYAAEEGLRITGQAMEGGGFEAASKNKLAVV |
| | | | | | | | | | RREPVGIVLAIAPFNYPVNLSASKIAPALIAGNVVM |
| | | | | | | | | | FKPPTQGSISGLLLAKAFEEAGIPAGVFNTITGRGSE |
| | | | | | | | | | IGDYIIEHKEVNFINFTGSTPIGERIGRLAGMRPIMLE |
| | | | | | | | | | LGGKDAALVLEDADLEHAAKQIVAGAFSYSGQRC |
| | | | | | | | | | TAIKRVIVLESVADKLATLLQEEVSKLTVGDPFDN |
| | | | | | | | | | ADITPVIDNASADFIWSLIEDAQEKEAQALTPIKRE |
| | | | | | | | | | GNLLWPVLFDQVTKDMKVAWEEPFGPVLPIIRVAS |
| | | | | | | | | | VEEAIAFANESEFGLQSSVFTNDFKKAFEIAEKLEV |
| | | | | | | | | | GTVHINNKTQRGPDNFPFLGVKGSGAGVQGIKYSI |
| | | | | | | | | | EAMTNVKSIVFDVK |
| 38 | peg.0401 | n/a | 0 | 5 | unknown | | | NA | LANNRKTETLGVSYLSTFIDKHELLQSYFESNDKTP |
| | | | | | | | | | VWDGEIHVLKSPSEKKDEILGKVPVQIKTTRQKKD |
| | | | | | | | | | VLKSFSLDTRDLELYKPNGGVVLFVVWLNEDNGL |
| | | | | | | | | | RDIYYKSLPPLSIKNLLKKSKLKNKSTNRKKLSIEIF |
| | | | | | | | | | KLDEKKMYPMLVDFINNSQKQYSFINVEGISVEDIP |
| | | | | | | | | | DDKTLKFYFYGQEKEEIFNYQEEHDLFIYYLDPITG |
| | | | | | | | | | IEIPLENTIKIVETEEETDLIIKIGDYVFQDVKRHRFP |
| | | | | | | | | | DGSVQLHFGESFTMSFDIKKKQFKFNYTRPDLLSK |
| | | | | | | | | | AIKCTQVFQELGKIGYFTLNGNKIELDERSIKDISSL |
| | | | | | | | | | DLEADIKGLLKISNFMKKMGIQKDVDLSCFDKQSQ |
| | | | | | | | | | RNLNILYSGLVLKKKVALNYNESKLLHLNIANIHII |
| | | | | | | | | | TLYSFLSDKNGTMIDIFTETPWCREGETEDEDYLDI |
| | | | | | | | | | SIFEVFEPNDWLKIDNCKIDSVIASYQRLVDNKLKY |
| | | | | | | | | | EGADRTILKIVIAADMAEDKTKRELLLNWAQCLSD |
| | | | | | | | | | WNLKYSKNCEMAIINDLQIKSRVRKLNSKETETLT |
| | | | | | | | | | NILVNSNDNYELCFGSSVLLKSKPQADLFWNKLDN |
| | | | | | | | | | ETKERYKDFPIYTLYMKLS |
| 39 | peg.0403 | n/a | 0 | 5 | unknown | | | cell wall binding | MKKILLSTVALLSLVASLLANNPVSAQESSSQATYS |
| | | | | | | | | | KSSGSWIKSGNRWWYKHSDGSYTTNGWEKINGT |
| | | | | | | | | | WYYFDSEGWMKTGWIKEYGKWYYLDDSGAMKT |
| | | | | | | | | | GWCLVSGSWYYLNSSGVMQTGLQTINGKQYYLA |
| | | | | | | | | | AGGAMQTGWHNIGDDTYFFANSGENQNINRRALV |
| | | | | | | | | | LGETSTRAVPIADVNAMEKVFNNQNFSEVVRFPDR |
| | | | | | | | | | TKSEIIAKMQELFESSSEGDVNYLYFTCHGGRDGRI |
| | | | | | | | | | YIGSDGLAFSGWELASVLKQYKGKFVVMLDCCHA |
| | | | | | | | | | GTIISKDNTGEGNEGASTEYFDLDEFVSGFSNMDG |
| | | | | | | | | | NEKSGEMIDSKFLVLCSSRGAEYSSGGSLSLATKY |
| | | | | | | | | | WSLGSGWNPLQNSQAYLAADQNNNRRITLNELYT |
| | | | | | | | | | YSREQVLKQNSNQHIEVYPDNSQFVLFKK |
| 40 | peg.0430 | n/a | 0 | 5 | CHO | | | Converts N- | METKKIKNLKGQIIVSCQALEGEPLYTPNGGVMPL |
| | | | | | metabolism | | | acetylmannosamine- | LAKAAFQAGAKGIRANSVRDISEIKEEVDLPIIGIIK |
| | | | | | | | | 6-phosphate | RDYDGFEPFISATMKEIDELVSEGVDILALDCTNRS |
| | | | | | | | | (ManNAc-6-P) to N- | RPGYDNITDFIHDIKVKYPNQLLMADISTFEEGKVA |
| | | | | | | | | acetylglucosamine-6- | AESGVDFVGTTLSGYTPYSPKKDNPDFELVERLVK |
| | | | | | | | | phosphate (GlcNAc- | ELDVPVIAEGRISTPEQARKMLDLGAYAVVVGGAI |
| | | | | | | | | 6-P) (By similarity) | TRPLEIAKKFIEVV |
| 41 | peg.0485 | n/a | 0 | 5 | Bacteriocin | | | NA | MKFKKIVTSALVVLSLFSIMSPSIASVRVFADDVTT |
| | | | | | | | | | TEVSEISTQEQKQIDDVANVLEQMFRNGVNEKNFT |
| | | | | | | | | | EYVYKNFSQKDIALAENELETNINNPYDRVPWDE |
| | | | | | | | | | MGGCIAGKIRDEFFAMINVSLIVKYAQKKAWSELA |
| | | | | | | | | | KVVLRFVKANGLKTNIYIIAGQLAIWAVQCGLE |
| 42 | peg. 0514 | SP1423 | 0 | 5 | unknown | | | | MKNRFYYYQLLDEREEQLFNKAGSESFYICIALSLL |
| | | | | | | | | | SYIISVLAPSLFNSNMLLIVIIIGTFYFFNRARYLGVT |
| | | | | | | | | | YYSRFHFTILGCFFLTLAITALLMLQNYQFNIEIYQH |
| | | | | | | | | | NPLNFKYLSAWVITYVIYLPWVFIGNLGLKSYGEW |
| | | | | | | | | | AQKKFEQDMDELESGE |
| 43 | peg.0524 | SP1430 | 0 | 5 | DNA | | | DNA methylase n-4 | MTVLKGDNLEILKTIESSSIDLIYMDPPFFTQKTQKL |
| | | | | | methylation | | | n-6 domain protein | SNNKNIMYSFEDTWTSIEDYKEFLSVRLEECKRVL |
| | | | | | | | | | KNSGSIFVHCDKIANHHIRLILDNIFGADMFQSEIIW |
| | | | | | | | | | NYKRWSNSKKGLLNNHQNIYFYSKSKDFKFNTIFT |
| | | | | | | | | | EYSSTTNIDQILVERKRDGNSKTIYKVDNNGNYILA |
| | | | | | | | | | KEKNGVPLSDVWNIPFLNPKAKERVGYPTQKPILL |
| | | | | | | | | | LEQIIKIATDKNDIVLDPFCGSGTTLVASKILNRNY |
| | | | | | | | | | MGIDLSEEAINITQQRLENVIKTSSNLLNKGIEAYRT |
| | | | | | | | | | KTEEEENILKLLQAKIVQRNKGIDGFLPKHFQKKPI |
| | | | | | | | | | PIKIQKNNECLNESISLLQNAINSKKLDFGVVIKTHS |
| | | | | | | | | | DNLLFDFDTIPENIIVVDHFELTIEKWLSKSQQLL |
| 44 | peg.0529 | SP1449 | 0 | 5 | unknown | CppA | Yes | CppA protein | MNVNQIVRIIPTLKANNRKLNETFYIETLGMKALLE |
| | | | | | | | | | ESAFLSLGDQTGLEKLVLEEAPSMRTRKVEGRKKL |
| | | | | | | | | | ARLIVKVENPLEIEGILSKTDSIHRLYKGQNGYAFEI |
| | | | | | | | | | FSPEDDLILIHAEDDRASLVEVGEKPEFQTDLASISL |
| | | | | | | | | | SKFEISMELHLPTDIESFLESSEIGASLDFIPAQGQDL |
| | | | | | | | | | TVDNTVTWDLSMLKFLVNELDIASLRQKFEFTEYF |
| | | | | | | | | | IPKSEKFFLGKDRNNVELWFEEV |
| 45 | peg.0669 | SP1587 | 0 | 5 | transport | | Yes | Major Facilitator | MKSNRYIIAFAGVILHLMLGSTYAWSVYRNPIIEKT |
| | | | | | | | | | GWDQASVAFAFSLAIFCLGLSAAFMGRLVEKFGPR |
| | | | | | | | | | VMGSLSAFLYAGGNILTGFAIDRQELWLLYLAYGI |
| | | | | | | | | | LGGLGLGAGYITPVSTIIKWFPDKRGLATGLAIMGF |
| | | | | | | | | | GFASLLTSPIAQHLIAGVGLVETFYILGASYFIIMLL |
| | | | | | | | | | ASQFIKRPNEQELAILSASGKEKTDSLTQGMTANQ |
| | | | | | | | | | ALKSNRFYILWIIFFINIACGLGLISAASPMAQEMAG |
| | | | | | | | | | LSTSHAAVMVGVLGIFNGFGRLLWASLSDYIGRPL |
| | | | | | | | | | TFSILLLVNLFFSLSLWLFTDSVLFVVAMSILMTCY |
| | | | | | | | | | GAGFSLIPAYLSDIFGTKELAALHGYILTAWAMAG |
| | | | | | | | | | LAGPILLAETYKMAHSYTQTLFVFLILYSIALALSY |
| | | | | | | | | | YLGRSIKKESQKPLT |
| 46 | peg.0749 | SP1674 | 0 | 5 | regulation | | | transcriptional | MDKPDIATIIDSHFEEMTDLEQEIARYFLQAETITDD |
| | | | | | | | | regulator | LSSQQVTQKLHISQAALTRFAKKCGFTGYREFIFQY |
| | | | | | | | | | QHQAENQANQVSKHSPLTKRVLRSYSNMREQTQD |
| | | | | | | | | | LIDEIQLERIAQLIEDAERIYFFGTGSSGLVAREMKL |
| | | | | | | | | | RFMRLGVVCEALTDQDGFAWTTSIMDENCLVLGF |
| | | | | | | | | | SLSGSTPSILDSLLDAKEMGAKTVLFTSVPNKDSQT |
| | | | | | | | | | YTETVLVATHSQPSYIQRISAQLPMLFFIDLIYAYFL |
| | | | | | | | | | EINRESKEKIFNSYWENKKLNGYRRQKRVRKS |
| 47 | peg.0755 | SP1681 | 0 | 5 | CHO | | Yes | ABC transporter | MQSTEKKPLTAFTVISTIILLLLTVLFIFPFYWILTGA |
| | | | | | transport | | | (Permease) | FKSQPDTIVIPPQWFPKMPTMENFQQLMVQNPALQ |
| | | | | | | | | | WMWNSVFISLVTMFLVCATSSLAGYVLAKKRFYG |
| | | | | | | | | | QRILFAIFIAAMALPKQVVLVPLVRIVNFMGIHDTL |
| | | | | | | | | | WAVILPLIGWPFGVFLMKQFSENIPTELLESAKIDG |
| | | | | | | | | | CGEIRTFWSVAFPIVKPGFAALAIFTFINTWNDYFM |
| | | | | | | | | | QLVMLTSRNNLTISLGVATMQAEMATNYGLIMAG |
| | | | | | | | | | AALAAVPIVTVFLVFQKSFTQGITMGAVKG |
| 48 | peg.0760 | SP1686 | 0 | 5 | metabolism | | | oxidoreductase | MVKYGVVGAGYFGAELARYMQKNDGAEITLLYD |
| | | | | | | | | | PDNAEAIAEELGAKVASSLDELVSSDEVDCVIVATP |
| | | | | | | | | | NNLHKEPVIKAAQHGKNVFCEKPIALSYQDCREM |
| | | | | | | | | | VDACKENNVTFMAGHIMNFFNGVHHAKELINQGV |
| | | | | | | | | | IGDVLYCHTARNGWEEQQPSVSWKKIREKSGGHL |
| | | | | | | | | | YHHIHELDCVQFLMGGMPETVTMTGGNVAHEGE |
| | | | | | | | | | HFGDEDDMIFVNMEFSNKRFALLEWGSAYRWGEH |
| | | | | | | | | | YVLIQGSKGAIRLDLFNCKGTLKLDGQESYFLIHES |
| | | | | | | | | | QEEDDDRTRIYHSTEMDGAIAYGKPGKRTPLWLSS |
| | | | | | | | | | VIDKEMRYLHEIMEGAPVSEEFAKLLTGEAALEAI |
| | | | | | | | | | ATADACTQSMFEDRKVKLSEIVK |
| 49 | peg.0777 | SP1704 | 0 | 5 | transport | | | ABC transporter | MRLEIINGQKIYGKRPILNQLNLVFQSGKIYGLKGD |
| | | | | | | | | | NGSGKTVLLKILAGYIKLDKGKVLQDGKVYGVKN |
| | | | | | | | | | HYIQDAGILIEKVEFLSHLSLRENLELLRYFSSKVTE |
| | | | | | | | | | KRIAYWIQYYDLQEFEDIEYRHLSLGTKQKMALIQ |
| | | | | | | | | | AFISSPSILFLDEPMNALDEKSVRITKQVILSYLKKE |
| | | | | | | | | | NGLVILTSHISEDISDLCTDVLVVENGHIQM |
| 50 | peg.0854 | SP1795 | 0 | 5 | CHO | | | sucrose-6-phosphate | MGDKKYTVEKANRFIAENKHLVNTQYKPEEHFSA |
| | | | | | metabolism | | | hydrolase | EIGWINDPNGFVYFRGEYHLFYQFYPYDSVWGPM |
| | | | | | | | | | HWGHAKSKDLVTWEHLPVALAPDQDYDRNGCFS |
| | | | | | | | | | GSAIVKDDRLWLMYTGHIEEETGVRQVQNMAFSD |
| | | | | | | | | | DGIHFEKIEQNPVATGSDLPDELIAADFRDPKLFEK |
| | | | | | | | | | DGRYYSVVAAKHKNNVGCIVLLGSDNLVEWQFES |
| | | | | | | | | | IFLKGVEHQGFMWECPDYFELDGKDCLIMSPMRY |
| | | | | | | | | | QREGDSYHNINSSLLFTGKVDWGEKCFIPESVQEID |
| | | | | | | | | | HGQDFYAPQTLLDDQNRRILIAWMQTWGRTLPTH |
| | | | | | | | | | DQEHKWACAMTLPRILRLEDGKLRQFPVKKGQYQ |
| | | | | | | | | | IQIDKDCHYHLGNDIDYLEFGYDSNAQQVYIDRSH |
| | | | | | | | | | LIQKILGEEEQDTSRRYVDIEAKELEVVLDKNSIEIF |
| | | | | | | | | | VNQGEASLTATYYLTVPAELSRID |
| 51 | peg.0873 | n/a | 0 | 5 | metabolism | | | PEP | MKRLISANPSEILQMNAEELKQSILASEGRVVLSEN |
| | | | | | | | | phosphonomutase | VVTRETFVGDITNSEIARAFGADMILLNCVDVFEPK |
| | | | | | | | | family protein | IYALDSSGDDVIHRLHQLVACPIGVNLEPIDPSAKM |
| | | | | | | | | | LEETQEIVAGRVASVETLNRIEELGFDFVCLTGNPG |
| | | | | | | | | | TGVSNQEIIKAVQSAKENFSGLIIAGKMHGAGVNE |
| | | | | | | | | | PVAELSVAEQLLEAGADVILVPAVGTVPAFHDQEL |
| | | | | | | | | | REVVDLVHSKGGLVLSAIGTSQETSDTDTIKEIALR |
| | | | | | | | | | NKICGVDIQHIGDAGYGGLATVDNIYALSKAIRGV |
| | | | | | | | | | RHTVSRLARSVNR |
| 52 | peg.0875 | n/a | 0 | 5 | regulation | | | ROK family | MTLSKKQLQLRAKILETVYTLGPISRIEIATKTGITP |
| | | | | | | | | | ATTSSITNDLIKENILLELGEDEHDTSVGRKKILLDI |
| | | | | | | | | | QAKRFYYIGCELSEKHFTFALGDNLGNILKEEKEIV |
| | | | | | | | | | TKQLIQEKGNQLINQTLKQFLNNCSDYEIEAIGIALP |
| | | | | | | | | | GRYLDNYKITTNNPLWQHIDLEMIQSHFDKPLFFS |
| | | | | | | | | | NNVNCMAIGKRLFSRQQNDPNFAYFHFARGMHCS |
| | | | | | | | | | YIYDGNIYGKGNLMIGEIGHTVVSSEGEECSCGRK |
| | | | | | | | | | GCLQTFAGESWLIKKSKILYHQSPYSLLPSLVKNAD |
| | | | | | | | | | DIDIQVILTAYQLGDTGIITLIHQALLYLSQTILNISM |
| | | | | | | | | | MIDSQKIYLHSPLLTNQHIIQKLYSEMNYKPKLLYN |
| | | | | | | | | | RLPEVIIEPYNDFTAAHSAIALCLYHTILHS |
| 53 | peg.0896 | SP1830 | 0 | 5 | regulation | | | Plays a role in the | MLRLYLENEIIELSNNLETIWVSVLDKYEKLFEYLS |
| | | | | | | | | regulation of | NEERIELIKEDLIINESVLNVDKKGYELICLQHPVSY |
| | | | | | | | | phosphate uptake | DLRRIISVIKISTDIERIGDRIVEILKNLQIIQNNEILKK |
| | | | | | | | | | IISEIKILHEVIGLHMNRAISCYREEQSGCLDMVVIQ |
| | | | | | | | | | KQNEIEELSINMEKKIMNYIFEDDGNVSEVISALDII |
| | | | | | | | | | HHLDKIAHTTQAIYKWIMYRKYGNIN |
| 54 | peg.1022 | SP1933 | 0 | 5 | regulation | | | NA | MNVKKIMSIFQSFYVDVSIEELTLTLPISFVKRFEYT |
| | | | | | | | | | QMTFHKESFLLIKEKRRGSLSSFVTQARTMGEKAN |
| | | | | | | | | | MDVVLVFSKLSDSEKKQLLQARVPFVDFKGNLFFP |
| | | | | | | | | | PLGLVLNANDTEVPKELTPSEQLTWIAFLLTKGQK |
| | | | | | | | | | VVDVDLLSQVTGLPNSTIYRCLRTFKALYWLNKQ |
| | | | | | | | | | NKLYTYTVSKKELFLKSVSCLFNPIKKRILLPDGDI |
| | | | | | | | | | KQIKSVSNLLYGGAYALSHSTFLAETDENISYVIWQ |
| | | | | | | | | | RKFNQLSLPLSQHVLK |
| 55 | peg.1023 | SP1934 | 0 | 5 | unknown | | | NA | LVAKGSAEIVSIDIGTEEYALYRDLTRNHDNNKIIG |
| | | | | | | | | | KGEAASISLAKKHNGILGSNNLRDVKSYVEEFSLE |
| | | | | | | | | | HMTTGDILIEAFKA |
| 56 | peg.1076 | SP1989 | 0 | 5 | regulation | | | Transcriptional | MLIGQKIKEIRIEKGISRPDFCGDEQELTVRQLSRIES |
| | | | | | | | | regulator | GASQPSLPKLDYIARRLGVPVYSLMPDFSALPSAYL |
| | | | | | | | | | ELKYQILREPIYGKEEEYDKKEACLEEIYKTYFDNL |
| | | | | | | | | | PKEEQLACEVLQACLDTSRTRRPEYAELILEEHMS |
| | | | | | | | | | QIIEKEVYSTNDMLLIRLFFYQMLIRKDLAKFINQIE |
| | | | | | | | | | KLMLFLLEQKKVTQIENYFIIRDTLISGMCCLEKVG |
| | | | | | | | | | VTDCFNDYLSCLQEIMDKTQDYQKKPLVFMFLWK |
| | | | | | | | | | QALRVERDFSLAESFYQSSKTFAKLIGDGFLVKKLT |
| | | | | | | | | | EEWQEDVKKYL |
| 57 | peg.1118 | SP2031 | 0 | 5 | CHO | | Yes | L-ascorbate 6- | MPNVKEITRESWILATFPEWGTWLNEEIEEEVVPEG |
| | | | | | metabolism | | | phosphate lactonase | NFAMWWLGNCGTWIKTPAGANVVMDLWSNRGK |
| | | | | | | | | | STKKVKDMVRGHQMANMAGVRKLQPNLRVQPM |
| | | | | | | | | | VIDPFAINELDYYLVSHFHSDHIDPYTAAAILNNPK |
| | | | | | | | | | LEHVKFIGPYHCGRIWEGWGVPKERIIVVKPGDTIE |
| | | | | | | | | | LKDMKIHAVESFDRTCLVTLPVNGADETGGELAG |
| | | | | | | | | | LAVTDEEMAQKAVNYIFETPGGTIYHGADSHFSNY |
| | | | | | | | | | FAKHGKDFKIDVALNNYGENPVGIQDKMTSIDLLR |
| | | | | | | | | | MAENLRTKVIIPVHYDIWSNFMASTNEILELWKMR |
| | | | | | | | | | KDRLQYDFHPFIWEVGGKYTYPQDQHLVEYHHPR |
| | | | | | | | | | GFDDCFEQDSNIQFKALL |
| 58 | peg.1181 | SP2095 | 0 | 5 | signaling | | | Rhomboid family | MKEIFDRRYPVTSFFLLVTALVFLLMLVTAGGNFD |
| | | | | | | | | | RADTLFRFGAMYGPAIRLFPEQVWRLLSAIFVHIG |
| | | | | | | | | | WEHFIVNMLSLYYLGRQVEEIFGSKQFFFLYLLSG |
| | | | | | | | | | MMGNLFVFVFSPKSLAAGASTSLYGLFAAIIVLRY |
| | | | | | | | | | ATRNPYIQQLGQSYLTLFVVNIIGSVLIPGISLAGHI |
| | | | | | | | | | GGAVGGAFLAVIFPVRGEKRMYNTSQRLGAVVLF |
| | | | | | | | | | VGLAILLFYKGMGM |
| 59 | peg.1198 | SP2112 | 0 | 5 | Regulation | | Yes | transcriptional | MPVTIKDVAKAAGVSPSTVTRVIQNKSTISDETKK |
| | | | | | | | | regulator | RVRKAMKELNYHPNLNARSLVSSYTQVIGLVLPD |
| | | | | | | | | | DSDAFYQNPFFPSVLRGISQVASENHYAIQIATGKD |
| | | | | | | | | | EKERLNAISQMVYGKRVDGLIFLYAQEEDPLVKLV |
| | | | | | | | | | AEEQFPFLILGKSLSPFIPLVDNGNVQAGFDATEYFI |
| | | | | | | | | | KKGCKRIAFIGGSKKLFVTKDRLTGYEQALKHYKL |
| | | | | | | | | | TTDNNRIYFADEFLEEKGYKFSKRLFKHDPQIDAIIT |
| | | | | | | | | | TDSLLAEGVCNYIAKHQLDVPVLSFDSVNPKLNLA |
| | | | | | | | | | AYVDINSLELGRVSLETILQIINDNKNNKQICYRQLI |
| | | | | | | | | | AHKIIEK |
| 60 | peg.1221 | SP2132 | 0 | 5 | Unknown | | | Band 7 protein | MTEKLINSKPNGVFALILIELTIVLGIFIFIMGVGSEN |
| | | | | | | | | | IFGIIIGPLLIVIAGLAHAGLKVVKPQEALVLTLFGN |
| | | | | | | | | | YTGTIKEPGFYFVNPFSVAVNPANHTRLGQSGDVS |
| | | | | | | | | | TKSPFLGAKSSNDNDVNLEIGKKHISLKVMTLSNS |
| | | | | | | | | | RQKINDCLGNPVEIGIAVTWRVVDTAKAVFNVDN |
| | | | | | | | | | YKEYLSLQCDSSLRNIVRIYPYDVSPNVDTTGDGQ |
| | | | | | | | | | ADEGSLRGSSEIVANRIREEIQSRVEDAGLEILEARI |
| | | | | | | | | | TYLAYAPEIAAVMLQRQQASAIIDARKMIVDGAVG |
| | | | | | | | | | MVEMALERLNEGELVELDEERKAAMVSNLLVVLC |
| | | | | | | | | | GNHDAQPIVNTGSLY |
| 61 | peg.1233 | SP2144 | 0 | 5 | CHO | | | Conserved Protein | MIYSKEIVREWLDEVAERAKDHPEWVDVFERCYT |
| | | | | | metabolism | | | | DTLDNTVEILEDGSTFVLTGDIPAMWLRDSTAQLR |
| | | | | | | | | | PYLHVAKRDALLRQTIAGLVKRQMTLVLKDPYAN |
| | | | | | | | | | SFNIEENWKGHHETDHTDLNGWIWERKYEVDSLC |
| | | | | | | | | | YPLQLSYLLWKETGETSQFDETFVAATKEILHLWT |
| | | | | | | | | | VEQDHKNSPYRFVRDTDRKEDTLVNDGFGPDFAV |
| | | | | | | | | | TGMTWSAFRPSDDCCQYSYLIPSNMFAVVVLGYV |
| | | | | | | | | | QEIFAALNLADSQSVIADAKRLQDEIQEGIKNYAYT |
| | | | | | | | | | TNSKGEKIYAFEVDGLGNASIMDDPNVPSLLAAPY |
| | | | | | | | | | LGYCSVDDEVYQATRRTILSSENPYFYQGEYASGL |
| | | | | | | | | | GSSHTFYRYIWPIALSIQGLTTRDKAEKKFLLDQLV |
| | | | | | | | | | ACDGGTGVMHESFHVDDPTLYSREWFSWANMMF |
| | | | | | | | | | CELVLDYLDIR |
| 62 | peg.1236 | SP2148 | 0 | 5 | Amino acid | | Yes | Arginine dihydrolase | MSSHPIQVFSEIGKLKKVMLHRPGKELENLLPDYL |
| | | | | | metabolism | | | | ERLLFDDIPFLEDAQKEHDAFAQALRDEGIEVLYLE |
| | | | | | | | | | QLAAESLTSPEIRDQFIEEYLDEANIRDRQTKVAIRE |
| | | | | | | | | | LLHGIKDNQELVEKTMAGIQKVELPEIPDEAKDLT |
| | | | | | | | | | DLVESDYPFAIDPMPNLYFTRDPFATIGNAVSLNH |
| | | | | | | | | | MFADTRNRETLYGKYIFKYHPIYGGKVDLVYNRE |
| | | | | | | | | | EDTRIEGGDELVLSKDVLAVGISQRTDAASIEKLLV |
| | | | | | | | | | NIFKKNVGFKKVLAFEFANNRKFMHLDTVFTMVD |
| | | | | | | | | | YDKFTIHPEIEGDLHVYSVTYENEKLKIVEEKGDLA |
| | | | | | | | | | ELLAQNLGVEKVHLIRCGGGNIVAAAREQWNDGS |
| | | | | | | | | | NTLTIAPGVVVVYDRNTVTNKILEEYGLRLIKIRGS |
| | | | | | | | | | ELVRGRGGPRCMSMPFEREEV |
| 63 | peg.1272 | SP2182 | 0 | 5 | unknown | | | | MSKMFKCMCLLVMSVFCMSSISPSLVVFADDSLSA |
| | | | | | | | | | NSKVVSGEAQFENGSSVRFGDTQVNILSDEVLEVV |
| | | | | | | | | | NPDGSVDTIERRADGVYINGAFYMAYQKNEIDLNI |
| | | | | | | | | | SFRSYDPNVWNYVNTIHGNKQANTFANFMTGAGI |
| | | | | | | | | | SYMIGRIGALLGGPWGAIIGGAYFGIQAYQSYLDS |
| | | | | | | | | | QSPYPYYITSTYIHVAQRKWKFITEYYRNSNYTGY |
| | | | | | | | | | VKTVTTYVNF |
| 64 | peg.1277 | SP2187 | 0 | 5 | Regulation | | | M protein trans- | MEKAECGQFSILSFLLQESQTTVKAVMEETGFSKA |
| | | | | | | | | acting positive | TLTKYVTLLNDKALDSGLELTIHSEDENLRLSIGAA |
| | | | | | | | | regulator | TKGRDIRSLFLESAVKYQILVYLFYHQQFLAHQLA |
| | | | | | | | | | QELVISEATLGRHLAGLNQILSEFDLSIQNGRWRGP |
| | | | | | | | | | EHQIRYFYFCLFRKVWSSQEWEGHMQKPERKQEI |
| | | | | | | | | | ANLEEICGASLSVGQKLDLVLWAHISQQRLRVNAC |
| | | | | | | | | | QFQVIEEKMRGYFDNIFYLRLLRKVPSFFAGQHIPL |
| | | | | | | | | | GVEDGEMMIFFSFLLSHRILPLHTMEYILGFGGQLA |
| | | | | | | | | | DLLTQLIQEMKKEELLGDYTEDHVTYELSQLCAQV |
| | | | | | | | | | YLYKGYILQDRYKYQLENRHPYLLMEHDFKETAE |
| | | | | | | | | | EIFHALPAFQQGTDLDKKILWEWLQLIEYMAENGG |
| | | | | | | | | | QHMRIGLDLTSGFLVFSRMAAILKRYLEYNRFITIE |
| | | | | | | | | | AYDPSRHYDLLVTNNPIHKKEQTPVYYLKNDLDM |
| | | | | | | | | | EDLVAIRQLLFT |
| 65 | peg.1386 | SP0045 | 0 | 5 | CHO | | Yes | phosphoribosylformyl | MDKRIFVEKKADFQVKSESLVRELQHNLGLSSLKS |
| | | | | | metabolism | | | glycinamidine | IRIVQVYDVFDLAEDLFAPAEKHIFSEQVTDHVLDE |
| | | | | | | | | synthase | VSVQADLANYAFFAIESLPGQFDQRAASSQEALLL |
| | | | | | | | | | LGSSSDVTVNTAQLYLVNKDIDVTELEAVKNYLLN |
| | | | | | | | | | PVDSRFKDITTGIAKQEFSESDKTIPKLTFFESYTAE |
| | | | | | | | | | DFARYKAEQGMAMEVDDLLFIQDYFKSIGRVPTET |
| | | | | | | | | | ELKVLDTYWSDHCRHTTFETELKHIDFSASKFQKQ |
| | | | | | | | | | LQSTYDKYIAMREELGRSEKPQTLMDMATIFGRYE |
| | | | | | | | | | RANGRLDDMEVSDEINACSVEIEVDVDGVKEPWL |
| | | | | | | | | | LMFKNETHNHPTEIEPFGGAATCIGGAIRDPLSGRS |
| | | | | | | | | | YVYQAMRISGAGDITAPISETRAGKLPQQVISKTAA |
| | | | | | | | | | HGYSSYGNQIGLATTYVREYFHPGFVAKRMELGA |
| | | | | | | | | | VVGAAPKGNVVREKPEAGDVIILLGGKTGRDGVG |
| | | | | | | | | | GATGSSKVQTVESVETAGAEVQKGNAIEERKIQRL |
| | | | | | | | | | FRNGNVTRLIKKSNDFGAGGVCVAIGELADGLEID |
| | | | | | | | | | LNKVPLKYQGLNGTEIAISESQERMAVVVRPEDVD |
| | | | | | | | | | TFVAECNKENIDAVVVATVTEKPNLVMHWNGETI |
| | | | | | | | | | VDLERRFLDTNGVRVVVDAKVVDKDVKLPEERQT |
| | | | | | | | | | SAETLESDTLTVLSDLNHASQKGLQTIFDCSVGRST |
| | | | | | | | | | VNHPLGGRYQLTPTEASVQKLPVQHGVTHTASVIA |
| | | | | | | | | | QGFNPYVAEWSPYHGAAYAVIEATARLVAAGAN |
| | | | | | | | | | WSKARFSYQEYFERMDKQAERFGQPVAALLGSIE |
| | | | | | | | | | AQIQLGLPSIGGKDSMSGTFEELTVPPTLVAFGVTT |
| | | | | | | | | | ADSRKVLSPEFKAVGENIYYIPGQALSAEIDFDLIK |
| | | | | | | | | | KNFAQFEASQADHKVTSASAVKYGGVVESLALAT |
| | | | | | | | | | FGNYIGAEVTLPELKTALTAQLGGFVFTSPEEIAGV |
| | | | | | | | | | EKIGQTKADFTLTVNGVKLDGHKLDSAFQGTLEEV |
| | | | | | | | | | YPTEFTQAKELEEVPAVASDVVIKAKEKVEKPVVY |
| | | | | | | | | | IPVFPGTNSEYDSAKAFEKEGAEVNLVPFVTLNEEA |
| | | | | | | | | | IVKSVETMVDNIDKTNILFFAGGFSAADEPDGSAKF |
| | | | | | | | | | IVNILLNEKVRVAIDSFIARGGLIIGICNGFQALVKS |
| | | | | | | | | | GLLPYGNFEDANSTSPTLFYNDANQHVAKMVETRI |
| | | | | | | | | | ANTNSPWLAGVQVGDIHAIPVSHGEGKFVVTAEEF |
| | | | | | | | | | AELRDNGQIFSQYVDFNGKPSMDSKYNPNGSVHAI |
| | | | | | | | | | EGITSKNGQIIGKMGHSERYEDGLFQNIPGNKDQH |
| | | | | | | | | | LFASAVKHFTGK |
| 66 | peg.1387 | SP0046 | 0 | 5 | CHO | | | glutamine | MTYEVKSLNEECGVFGIWGHPDAAKLTYFGLHSL |
| | | | | | metabolism | | | phosphoribosylpyro- | QHRGQEGAGILSNDQGQLKRHRDMGLLSEVFRNP |
| | | | | | | | | phosphate | ANLDKLTGAGAIGHVRYATAGEASVDNIQPFLFRF |
| | | | | | | | | amidotransferase | HDMQFGLAHNGNLTNAASLKKELEQRGAIFSATS |
| | | | | | | | | | DSEILAHLIRRSHNPSLMGKIKEALSLVKGGFAYIL |
| | | | | | | | | | LFEDKLIAALDPNGFRPLSIGKMANGAVVVSSETC |
| | | | | | | | | | AFEVIGVEWICDLKPGEIVIIDDEGIQYDSYTDDTQL |
| | | | | | | | | | AICSMEYIYFARPDSNIHGVNVHTARKRMGAQLAR |
| | | | | | | | | | EFKHEADIVVGVPNSSLSAAMGFAEESGLPNEMGL |
| | | | | | | | | | IKNQYTQRTFIQPTQELREQGVRMKLSAVSGVVKG |
| | | | | | | | | | KRVVMVDDSIVRGTTSRRIVQLLKEAGATEVHVAI |
| | | | | | | | | | GSPALAYPCFYGIDIQTRQELIAANHTVEETRQIIGA |
| | | | | | | | | | DSLTYLSIDGLIESIGIETDAPNGGLCVAYFDGDYPT |
| | | | | | | | | | PLYDYEEDYRRSLEEKTSFYK |
| 67 | peg.1419 | SP0082 | 0 | 5 | adhesin | | | cell wall surface | MKFNPNQRYTRWSIRRLSVGVASVVVASGFFVLV |
| | | | | | | | | anchor family protein | GQPSSVRADVVNPTPGQVLPEETSGTKEGDLSEKP |
| | | | | | | | | | GDTVLTQAKPEGVTGNTNSLPTPTERTEVSEETNSS |
| | | | | | | | | | SLDTLFEKDEEAQKNPELTDVLKEAVDTDDVDGT |
| | | | | | | | | | QASPAETTPEQVKGGVKENTKDSIDVPAAYLEKAE |
| | | | | | | | | | GKGPFTAGVNQVIPYELFAGDGMLTRLLLKASDN |
| | | | | | | | | | APWSDNGTAKNPALPPLEGLTKGKYFYEVDLNGN |
| | | | | | | | | | TVGKQGQALIDQLRANGTQTYKATVKVYGNKDG |
| | | | | | | | | | KADLTNLVATKNVDININGLVAKETVEKAVKDNV |
| | | | | | | | | | KDSIDVPAAYLEKAKGEGPFTAGVNHVIPYELFAG |
| | | | | | | | | | DGMLTRLLLKASDKAPWSDNGDAKNPALSPLGEN |
| | | | | | | | | | VKTKGQYFYQVALDGNVAGKEKQALIDQFRANG |
| | | | | | | | | | TQTYSATVNVYGNKDGKPDLDNIVATKKVTINING |
| | | | | | | | | | LISKETVQKAVADNVKDSIDVPAAYLEKAKGEGPF |
| | | | | | | | | | TAGVNHVIPYELFAGDGMLTRLLLKASDKAPWSD |
| | | | | | | | | | NGDAKNPALSPLGENVKTKGQYFYQVALDGNVV |
| | | | | | | | | | GKEKQALIDQFRANGTQTYSATVNVYGNKDGKPD |
| | | | | | | | | | LDNIVATKKVTININGLISKETVQKAVADNVKDSID |
| | | | | | | | | | VPAAYLEKAKGEGPFTAGVNHVIPYELFAGDGML |
| | | | | | | | | | TRLLLKASDKAPWSDNGDAKNPALSPLGENVKTK |
| | | | | | | | | | GQYFYQVALDGNVVGKEKQALIDQFRANGTQTYS |
| | | | | | | | | | ATVNVYGNKDGKPDLDNIVATKKVTININGLISKE |
| | | | | | | | | | TVQKAVADNVKDSIDVPAAYLEKAKGEGPFT AGV |
| | | | | | | | | | NHVIPYELFAGDGMLTRLLLKASDKAPWSDNGDA |
| | | | | | | | | | KNPALSPLGENVKTKGQYFYQVALDGNVAGKEK |
| | | | | | | | | | QALIDQFRANGTQTYSATVNVYGNKDGKPDLDNI |
| | | | | | | | | | VATKKVTIKINVKETSDTANGSLSPSNSGSGVTPM |
| | | | | | | | | | NHNHATGTTDSMPADTMTSSTNTMAGENMAASA |
| | | | | | | | | | NKMSDTMMSEDKAMLPNTGETQTSMASIGFLGLA |
| | | | | | | | | | LAGLLGGLGLKNKKEEN |
| 68 | peg.1433 | SP0097 | 0 | 5 | unknown | | | | MKQEWFESNDFVKTTSKNKPEEQAQEVADKAEET |
| | | | | | | | | | IADFDTPIEKNTQLEEEVSQAEVELESQQEEKIEAPE |
| | | | | | | | | | DSEARTGIEEKKASNSTEEEPDLSKETEKVTIAEES |
| | | | | | | | | | QEALPQQKATTKEPLLISKSLESPYIPDQAPKSRDK |
| | | | | | | | | | WKEQVLDFWSWLVEAIKSPTSKLETSITHSYTAFL |
| | | | | | | | | | LLILFSASSFFFSIYHIKHAYYGHIASINSRFPEQLAP |
| | | | | | | | | | LTLFSIVSILVATTLFFFSFLLGSFVVRRFIHQEKDW |
| | | | | | | | | | TLDKVLQQYSQLLAIPIFLTAIASFFAFFDSLRFTAL |
| | | | | | | | | | LCVISIGIILLASLHIITRPSQASETDSFYQLFLSVLVN |
| | | | | | | | | | GVIILLFFVAEVALIGDYLRILAFL |
| 69 | peg.1437 | SP_0101 | 0 | 5 | transport | | | Major Facilitator | MKKQSLFFVPGIILIGVSLRTPFTVLPIILGNISQGLE |
| | | | | | | | | Superfamily | VEVSSLGVLTSLPLLMFTLFSPFSTQLAQKIGLEHLF |
| | | | | | | | | | TYSLFFLTIGSLIRLINLPLLYLGTLMVGASVAVINV |
| | | | | | | | | | LLPSLIQANQPKKIGFLTTLYVTSMGIATALASYLA |
| | | | | | | | | | VPITQASSWKGLILLLTLLCLATFLVWLPNHRYNH |
| | | | | | | | | | RLAPQTKQKSQIKVMRNKQVWAIIIFSGFQSLIFYT |
| | | | | | | | | | AMTWLPTMSIHAGLSSHEAGLLTSILSLISIPFSMTIP |
| | | | | | | | | | SLTTSLSTRNRQLMLTLVSLAGVVGISMLFFPINNFI |
| | | | | | | | | | YWLAIHLLIGTATSALFPYLMVNFSLKTSAPEKTA |
| | | | | | | | | | QLSGLSQTGGYILAAFGPTLFGYSFDLFHSWVPSV |
| | | | | | | | | | AALLLIDILMTVALFTVDRADKIL |
| 70 | peg.1486 | n/a | 0 | 5 | unknown | | | | MKIKEQTRKLAAGCSKHCFEVADRTDEVSSKRCFE |
| | | | | | | | | | VVDRTDEVSNIYTAK |
| 71 | peg.1515 | SP_0177 | 0 | 5 | Metabolism | | | riboflavin synthase, | MFTGIIEEIGKVERIQKDSRNCKLSIKASKILTDIHLG |
| | | | | | | | | subunit alpha | DSIAVNGICLTVTHFNHQSFTVDVMNETWSRTALT |
| | | | | | | | | | LLKHGSEVNLERALSVNGRLGGHVVTGHIDGTGKI |
| | | | | | | | | | SSIKKDDNAVWYQINTQKEILDLIVEKGSITIDGISL |
| | | | | | | | | | TVAKVSKVNFSVSVIPHTLEQTILKSKQVGSTVNLE |
| | | | | | | | | | NDILGKYVQKLMDNSPKSEISKELLYQNGF |
| 72 | peg.1580 | SP_0247 | 0 | 5 | Regulation | | | regulator | MNQDRNKLLSKIAYLYYIENLNQSQIAAKLGIYRT |
| | | | | | | | | | SISRMLTEARNAGIVKIEIENFDTNMFKLENYVKEK |
| | | | | | | | | | YGLESLEIIPNEFDDNPTILSERISQVAAGVLRNLID |
| | | | | | | | | | DNMKIGFSWGKSLSNLVDLIHSKSVRNVHFYPLAG |
| | | | | | | | | | GPSHIHAKYHVNTLIYEMSRKFHGECTFMNATIVQ |
| | | | | | | | | | ENKLLADGILQSRYFENLKNSWKDLDIAVVGIGDF |
| | | | | | | | | | SNKGKHQWLDMLTEDDFKELTKVKTVGEICCRFF |
| | | | | | | | | | DSKGKEVYENLQERTIAISLEDLKNIPQSLAVAYGD |
| | | | | | | | | | TKVSSILSVLRANLVNHLITDKNTILKVLEEDGDLT |
| | | | | | | | | | FREILGE |
| 73 | peg.1643 | n/a | 0 | 5 | CHO | | | malate | MVENVGQLAIEQAKANGGKLEVVSKVTVNNRHD |
| | | | | | metabolism | | | dehydrogenase | LSIAYSPGVASVSSAIAENADLAYTLTTKKNTVAV |
| | | | | | | | | (Oxaloacetate- | VSDGTAVLGLGDIGPAAAMPVMEGKAALFKRFAD |
| | | | | | | | | decarboxylating) | VDAVPIVLDTKDPEEIIRIVKAMAPTFGGVNLEDIS |
| | | | | | | | | | APRCFEIEQRLIEECDIPIFHDDQHGTAIVVLAGLFN |
| | | | | | | | | | SLKLAQKELDKVKIVVNGGGSAGLSITRKLLAAGA |
| | | | | | | | | | TDVTVVDRFGIISEEDHDKLAPHHQDIAKVTNRRL |
| | | | | | | | | | ASGSLADALEDADVFLGVSAPGALNPEWIAKMND |
| | | | | | | | | | KPIIFAMANPTPEIFPDEALAAGAYIVGTGRSDFPN |
| | | | | | | | | | QINNVLAFPGIFRGALDARAKKITVEMQIAAAKGIA |
| | | | | | | | | | SLIPDSELSPTNIIPDPFQEGVAKVVAKSVSDAVKD |
| 74 | peg.1673 | SPO324 | 0 | 5 | CHO | | | PTS System | MYPALKKHYGEDQEGFYQALEENCEFYNTNPHFL |
| | | | | | transport | | | | PFITSLHLVMLENGRPTKETRSIKMALMGPLAGIGD |
| | | | | | | | | | SLSQFCLAPLFSTIAASFAQEGLVVGPILFFLAMNTI |
| | | | | | | | | | LTAIKLSTGLYGYKLGTTVIDKLSEQMATISRIANII |
| | | | | | | | | | GVTVIAGLAATSVKIMVPITFAAGEVKADAKQSIV |
| | | | | | | | | | SIQGMLDKVAPALLPALFTLLVYYLIKEKKWTTYK |
| | | | | | | | | | LVILTVIIGIIGSWLKIIA |
| 75 | peg.1719 | SPO374 | 0 | 5 | unknown | | Yes | NA | MSKKRRNRHKKEGQEPQFDFDEAKELTVGQAIRK |
| | | | | | | | | | NEEVEAGVLPEDSILDKYVKQHRDEIEADKFATRQ |
| | | | | | | | | | YKKEEFVETQSLDDLIQEMREAVEKSEASSEEVPSS |
| | | | | | | | | | EDILLPLPLDDEEQGLDPLLLDDENPTEMTEEVEEE |
| | | | | | | | | | QNLSRLDQEDSEKKSKKGFILTVLALVSVIICVSAY |
| | | | | | | | | | YVYRQVARSTKEIETSQSTTANQSDVDDFNTLYDA |
| | | | | | | | | | FYTNSNKTALKNSQFDKLSQLKTLLDKLEGSREHT |
| | | | | | | | | | LAKSKYDSLATQIKAIQDVNAQFEKPAIVDGVLDT |
| | | | | | | | | | NAKAKSDAKFTDIKTGNTELDKVLDKAISLGKSQQ |
| | | | | | | | | | TSTSSSSSSQTSSSSSSQASSNTTSEPKPSSSNDTRSS |
| | | | | | | | | | RSEVNMGLSSAGVAVQRSASRVAYNQSAIDDSNN |
| | | | | | | | | | SAWDFADGVLEQILATSRSRGYITGDQYILERVNIV |
| | | | | | | | | | NGNGYYNLYKPDGTYLFTLNCKTGYFVGNGAGH |
| | | | | | | | | | ADDLDY |
| 76 | peg.1726 | SPO38O | 0 | 5 | bacteriocin | | | | VTIRTTHTYEYQYSLLFGDAGYLWLLLHLFSISKN |
| | | | | | | | | | QYYLQLANVTAKKLIENYDTLEEIDFALGKSGVLL |
| | | | | | | | | | SLIKYYQFTNDNTLKTFIHNSIWGNLSLFPTKRYSQ |
| | | | | | | | | | RKHFRL |
| 77 | peg.1825 | SP0482 | 0 | 5 | Transport | | | UPF0397 protein | MEIKFTIKQVVAVGIGAALFVVIGMINIPTPVPNTSI |
| | | | | | | | | | QLQYAVQALLSIIFGPIIGLLVGLIGHAIKDSLAGYG |
| | | | | | | | | | LWWTWIIASGLFGLVVGLFRKYVRVINGVFDWKD |
| | | | | | | | | | ILIFNLIQLLANALVWGVLAPLGDVVIYQEAAEKVF |
| | | | | | | | | | AQGIVAGIANGVSVAIAGTLLLLAYAGTQTRAGSL |
| | | | | | | | | | KKD |
| 78 | peg.1869 | SP0539 | 0 | 5 | Bacteriocin | | | | MNTKMMEQFHEMDITMLSSIEGGKNNWQTNVLE |
| | | | | | | | | | GGGAAFGGWGLGTAICAASGVGAPFMGACGYIGA |
| | | | | | | | | | KFGVDLWAGVTGATGGF |
| 79 | peg.1908 | SP_0575 | 0 | 5 | regulation | | | type iii restriction | LLSTGKYIGEGFDLPQLDTLILAAPFSWKNNLIQYS |
| | | | | | | | | protein res subunit | GRIHRNYKDKSLVRIFDYVDIHVPYLEKMFQKRQV |
| | | | | | | | | | AYRKMDYRAIEGEEKQFVYVDSRYEKVLIEDLAG |
| | | | | | | | | | ERQECLLILPYVHQTKLMNFLKEFRISQIEICIPETV |
| | | | | | | | | | ANKAWLDQLKSQKIKVSFTQSKIVTPILLVNKTIV |
| | | | | | | | | | WYDAMPLLGKVDEMTILRLESASIVSELVAGLR |
| 80 | peg.1937 | SP0607 | 0 | 5 | Amino acid | | | amino acid AbC | MESILEVLTPDNLVFIFKGFGLTLYISLIAIILSTIIGT |
| | | | | | transport | | | transporter | VLAVMRNGKNPILRIISSIYIEFVRNVPNLLWIFTIFL |
| | | | | | | | | | VFKMKSTPAGITAFTLFTSAALAEIIRGGLNAVDKG |
| | | | | | | | | | QYEAGMSQGFTSAQILYYIILPQAIRKMLPAIISQFV |
| | | | | | | | | | TVIKDTSLLYSVIALQELFGASQILMGRYFEPEQVF |
| | | | | | | | | | SLYILIALIYFSFNLAISSLSHMLAKRWQQAAE |
| 81 | peg.1949 | SP0620 | 0 | 5 | Amino acid | | | ABC transporter | MKKFSLLLAILPFLVACGNQATPKETSAQKTIVLAT |
| | | | | | transport | | | substrate-binding | AGDVPPFDYEDKGNLTGFDIEVLKAVDEKLSDYEI |
| | | | | | | | | protein | QFQRTAWESIFPGLDSGHYQAAANNLSYTKERAE |
| | | | | | | | | | KYLYSLPISNNPLVLVSNKKNPLTSLDQIAGKTTQE |
| | | | | | | | | | DTGTSNAQFINNWNQKHTDNPATINFSGEDIGKRIL |
| | | | | | | | | | DLANGEFDFLVFDKVSVQKIIKDRGLDLSVVDLPS |
| | | | | | | | | | ADSPSNYIVFSSDQKEFKEQFDKALKELYQDGTLE |
| | | | | | | | | | KLSNTYLGGSYLPDQSQLQ |
| 82 | peg.1997 | SP0667 | 0 | 5 | adhesin | CbpL | | surface protein | MNKRLFLKMSLVTLPILALFSQPVLAEENIHFSSCK |
| | | | | | | | | | EAWANGYSDIHEGEPGYSAKLDRDHDGVACELKN |
| | | | | | | | | | APKGAFKAKQSTVAQTNTSSATTSGWVEQDGAW |
| | | | | | | | | | YYFDENGNPVKNAWQGNYYLKSDGKMAQGEWI |
| | | | | | | | | | YDSSYQAWYYLTSDGSYAYSTWQGNYYLKSDGK |
| | | | | | | | | | MAANEWVDGGRYYVGADGVWKEGQASTASSSN |
| | | | | | | | | | DSNSEYSAALGKAKSYNSLFHMSKKRMYRQLTSD |
| | | | | | | | | | FDKFSNDAAQYAIDH* |
| 83 | peg.2043 | SP_0717 | 0 | 5 | Nucleic | | | 4-methyl-5-beta- | MTSLKLLKEKAPLVICITNDVVKNFTANGLVALGA |
| | | | | | acid | | | hydroxy ethylthiazole | SPAMSEFPADLEDLLKYAGGLLINIGTLTDENWKL |
| | | | | | metabolism | | | kinase | YQAALKIAEKYNVPAVLDPVACGAGEYRKKVAD |
| | | | | | | | | | DLINNYKLAAIRGNAGEIASLVGIDVASKGVDSAG |
| | | | | | | | | | VDNIDEIALAANEKFNIPIVVTGEVDAIAVNGEVVT |
| | | | | | | | | | IHNGSAMMPKVIGTGCLLGAVVASFIGLEKGQELK |
| | | | | | | | | | SLETAMLVYNIAGEIAEKRPNGHLPGTFKAEFINAL |
| | | | | | | | | | YEITDEDVKEFKRVK |
| 84 | peg.2093 | SP_0770 | 0 | 5 | Metal | | Yes | ABC transporter | MSILEVKNLSHGFGDRAIFEDVSFRLLKGEHIGLVG |
| | | | | | transport | | | | ANGEGKSTFMSIVTGKMLPDEGKVEWSKYVTAGY |
| | | | | | | | | | LDQHSVLAERQSVRDVLRTAFDELFKAEARINDLY |
| | | | | | | | | | MKMAEDGADVDALMEEVGELQDRLESRDFYTLD |
| | | | | | | | | | AKIDEVARALGVMDFGMDTDVTSLSGGQRTKVLL |
| | | | | | | | | | AKLLLEKPDILLLDEPTNYLDAEHIDWLKRYLQNY |
| | | | | | | | | | ENAFVLISHDIPFLNDVINIVYHVENQQLTRYSGDY |
| | | | | | | | | | YQFQEVYAMKKSQLEAAYERQQKEIADLKDFVAR |
| | | | | | | | | | NKARVATRNMAMSRQKKLDKMDIIELQSEKPKPS |
| | | | | | | | | | FDFKPARTPGRFIFQAKNLQIGYDRPLTKPLNLTFE |
| | | | | | | | | | RNQKVAIIGANGIGKTTLLKSLLGIISPIAGEVERGD |
| | | | | | | | | | YLELGYFEQEVEGGNRQTPLEAVWNAFPALNQAE |
| | | | | | | | | | VRAALARCGLTTKHIESQIQVLSGGEQAKVRFCLL |
| | | | | | | | | | MNRENNVLVLDEPTNHLDVDAKDELKRALKEYR |
| | | | | | | | | | GSILMVCHEPDFYEGWIDQIWDFNNLT |
| 85 | peg.2140 | SP0824 | 0 | 5 | Amino acid | | | ABC transporter, | MNFSFLPKYLPYFNYGAVVTILISICVIFLGTILGVV |
| | | | | | transport | | | permease protein | LAFGQRSKFKPLVWLANLYVWIFRGTPMMVQIMI |
| | | | | | | | | | AFALMHINAPTIQIGILGVDFSRLIPGILIISMNSGAY |
| | | | | | | | | | VSETVRAGINAVPKGQLEAAYSLGIRPKNAMRYVI |
| | | | | | | | | | LPQAVKNILPALGNEFITIIKDSSLLSAIGVMELWNG |
| | | | | | | | | | ATTVSTTTYLPLTPLLFAAFYYLIMTSILTVALKAFE |
| | | | | | | | | | KHMGQGDKK |
| 86 | peg.2160 | SP0843 | 0 | 5 | Nucleic | | | Catalyzes a reversible | MKLNKYIDHTLLKQDAKKKQIDSLLSEAREYGFAS |
| | | | | | acid | | | aldol reaction | VCVNPTWVEHAKKGLEGTDVKVCTVVGFPLGATT |
| | | | | | metabolism | | | between acetaldehyde | SAVKAFETKEAIQNGADEIDMVINVGALKSGNLAL |
| | | | | | | | | and D-glyceraldehyde | VESDIRAVVEASGDKLVKVIIEACLLTDQEKVVVC |
| | | | | | | | | 3-phosphate to | QLAQKAGADFVKTSTGFSTGGATIADVTLMRETV |
| | | | | | | | | generate | GSDMGVKAAGGARSYADALAFVEAGATRIGTSAG |
| | | | | | | | | 2-deoxy-D-ribose | VAILKGELADGDY |
| | | | | | | | | 5-phosphate (By | |
| | | | | | | | | similarity) | |
| 87 | peg.2177 | SP0858 | 0 | 5 | unknown | | | Membrane | MTELAKQLLELTYIVIGCQFLHTAYCSYKDKTNPV |
| | | | | | | | | | RLGTSAFWTLLSITFIGGSYMPNMSIGIIVILLSLLTL |
| | | | | | | | | | FKQVRIGTLPSLDEMKANIESNRLKNKIFIPVMLMA |
| | | | | | | | | | ILALVLAQMIPEFSKISISLAALFATISVLVITNSHPK |
| | | | | | | | | | SLLSENNRMTQQVSTSGIVPQLLGALGAIFTVAGV |
| | | | | | | | | | GDVISHLISGIVPSDSRFIGVLAYVLGMVLFTMIMG |
| | | | | | | | | | NAFAAFTVITAGVGVPFVFALGANPIVAGALAMTA |
| | | | | | | | | | GYCGTLLTPMAANFNALPAALMDMKDQNGVIKA |
| | | | | | | | | | QAGVALVMIVIHIFLMYFLAF |

*in van Opijnen and Camilli (2012) Genome Res. 22(12): 2541-2551
**Protein functions were predicted with the eggNOG pipeline, using HMMR on the bacteria database.
https://academic.oup.eom/mbe/article/34/8/2115/3782716

TABLE 2

| | | | | | | | Identified | | |
|---|---|---|---|---|---|---|---|---|---|
| | | | Mean | Number | | | in Murine | | |
| SEQ | BHN97 | TIGR4 | Proba- | of | Functional | | Naso- | | |
| ID | Locus | Locus | bility of | Donors | Classi- | | pharynx | | |
| NO: | ID | ID | Infection | Infected | fication | Name | TnSeq* | KEGG prediction** | BHN97 protein sequence |
| 88 | peg.0391 | SP_1282 | 0.04125 | 8 | transport | | | abc transporter atp- | MHYEHSRKGNHMIKINHLTITQNKDLRDLVSDLS |
| | | | | | | | | binding protein | MTIQDGEKVAIIGEEGNGKSTLLKILMGEALSDFTI |
| | | | | | | | | | KGNIQSDYQSLAYIPQKLPEELKKKTLHDYFFLDSI |
| | | | | | | | | | DLDYSILYRLAEELHFDSNRFASDQKIGNLSGGEAL |
| | | | | | | | | | KIQLIHELAKPFEILFLDEPSNDLDLETVDWLKGQIQ |
| | | | | | | | | | KTRQTVIFISHDEDFLSETADTIVHLRLVKHRKEAE |
| | | | | | | | | | TLVEHLDYDSYSEQRKANFAKQSQQAANNQRAY |
| | | | | | | | | | DKTMEKHRRVKQNVETALRATKDSTAGRLLAKK |
| | | | | | | | | | MKTVLSQEKRYEKTAQSMTQKPLEEEQIQLFFSDI |
| | | | | | | | | | QPLPASKVLVQLEKENLSIDDRVLVQKLQLTVRGQ |
| | | | | | | | | | EKIGIIGPNGVGKSTLLAKLKQLLNDKREISLGFMP |
| | | | | | | | | | QDYHKKLQLDLSPIAYISKTGEKEELQKIQSHLASL |
| | | | | | | | | | NFSYPEMQHQIRSLSGGQQGKLLLLDLVLRKPNFL |
| | | | | | | | | | LLDEPTRNFSPTSQPQIRKLFATYPGGLITVSHDRRF |
| | | | | | | | | | LKEICSIIYRMTEHGLKLVNLEDL |
| 89 | peg.0404 | n/a | 0.055 | 6 | unknown | | | Transcriptional | MENFGAVLKDIRISKNFRLKDLSCNEISESTISRFEN |
| | | | | | | | | regulator | GITKLSINHFYILLNRLGISFSEFEELVHCYYSKKEC |
| | | | | | | | | | LFEELEHAVNSSDIFLLQELVDKIELKQKQEKSLCN |
| | | | | | | | | | YHIKLIAEQQINRLANLPYNSSKCNELIKYLLSVDT |
| | | | | | | | | | WMEYELKLFYNSVFFMNTRTISLLYRIVIKKTRYFL |
| | | | | | | | | | KTNTGTHRIIPLYLFNLKLLLKNNLLGSAQFFIDDL |
| | | | | | | | | | ENLLTRQGYYFEKNYLLFLKGIYLIKTNQIELGKKE |
| | | | | | | | | | CSKAMRIFKEYNDSDTINELNQKFKLDLTI |
| 90 | peg.1576 | SP_0234 | 0.055 | 6 | Metal | | | ABC transporter | MKKKWMYYAACSSNESADDSSSDKGDGGSLVVY |
| | | | | | transport | | | | SPNSEGLIGATIPAFEEKYGIKIELIQAGTGELFKKLE |
| | | | | | | | | | SEKEVPVADVIFGGSYTQYATHGELFENYTSKEND |
| | | | | | | | | | NVIKEYQNTTGYSTPYTLDGSVLIVNPDLTKGMNI |
| | | | | | | | | | EGYNDLLKPELKGKIATADPANSSSAFAQLTNMLQ |
| | | | | | | | | | AQGGYKDDKAWSYVKDLFTLIDGKIGSSSSSVYK |
| | | | | | | | | | AVADGEMAVGLSYEDPAVKLLNDGANIKVVYPKE |
| | | | | | | | | | GTVFLPASAAIVKKAKNMENAKKFIDFIISQEVQDT |
| | | | | | | | | | LGTTTTNRPVRKNAKTSENMKPIDKIKTLTEDYDY |
| | | | | | | | | | VIKNKSDIVKKYNEVFTDIQSKQ |
| 91 | peg.1618 | SP_0287 | 0.055 | 6 | transport | | | Xanthine uracil | MDKLFKLKENGTDVRTEVLAGLTTFFAMSYILFVN |
| | | | | | | | | vitamin C permease | PQILSQTGMPAQGVFLATIIGAVAGTLMMAFYANL |
| | | | | | | | | | PYAQAPGMGLNAFFTFTVVFGLGYSWQEALAMVF |
| | | | | | | | | | ICGIISLVITLTNVRKMIIESIPNALRSAISAGIGVF |
| | | | | | | | | | LAYVGIKNAGLLKFTIDPGNYTVVGEGADKAQATIAA |
| | | | | | | | | | NSSAVPGLVSLNNPAVLVALAGLAITIFFVIKGIKG |
| | | | | | | | | | GIILSILTTTVLAIAVGLVDLSSIDFANNHVGAAFED |
| | | | | | | | | | LKTIFGAALGSEGLGALVSDTARLPETLMAILAFSL |
| | | | | | | | | | TDIFDTIGTLIGTGEKVGIVATNGENHQSAKLDKAL |
| | | | | | | | | | YSDLIGTTVGAIAGTSNVTTYVESAAGIGAGGRTG |
| | | | | | | | | | LTALVVAICFAISSFFSPLLAIVPTAATAPILIIVGIM |
| | | | | | | | | | MLGSLKNIHWDDMSEAVPAFFTSIFMGFSYSITQGI |
| | | | | | | | | | AVGFLTYTLTKLVKGQVKDVHAMIWILDALFILNY |
| | | | | | | | | | ISMAL |
| 92 | peg.1992 | SP_0662 | 0.055 | 6 | regulation | | | Histidine kinase | MKRSSLLVRMVISIFLVFLILLALVGTFYYQSSSSAI |
| | | | | | | | | | EATIEGNSQTTISQTSHFIQSYIKKLETTSTGLTQQT |
| | | | | | | | | | DVLAYAENPSQDKVEGIRDLFLTILKSDKDLKTVV |
| | | | | | | | | | LVTKSGQVISTDDSVQMKTSSDMMAEDWYQKAIH |
| | | | | | | | | | QGAMPVLTPARKSDSQWVISVTQELVDAKGANLG |
| | | | | | | | | | VLRLDISYETLEAYLNQLQLGQQGFAFIINENHEFV |
| | | | | | | | | | YHPQHTVYSSSSKMEAMKPYIETGQGYTPGHKSY |
| | | | | | | | | | VSQEKIAGTDWTVLGVSSLEKLDQVRSQLLWTLL |
| | | | | | | | | | GASVTSLLVCLCLVWFSLKRWIAPLKDLRETMLEI |
| | | | | | | | | | ASGAQNLRAKEVGAYELREVTRQFNAMLDQIDQL |
| | | | | | | | | | MVAIRSQEETTRQYQLQALSSQINPHFLYNTLDTII |
| | | | | | | | | | WMAEFHDSQRVVQVTKSLATYFRLALNQGKDLIC |
| | | | | | | | | | LSDEINHVRQYLFIQKQRYGDKLEYEINENVAFDN |
| | | | | | | | | | LVLPKLVLQPLVENALYHGIKEKEGQGHIKLSVQK |
| | | | | | | | | | QDSGLVIRIEDDGVGFQDAGDSSQSQLKRGGVGLQ |
| | | | | | | | | | NVDQRLKLHFGANYQMKIDSRPQKGTKVEIYINRI |
| | | | | | | | | | ETS |
| 93 | peg.0287 | SP_1175 | 0. | 9 | adhesin | His- | | (Histidine triad) | MKINKKYLVGSAAALILSVCSYELGLYQARTVKEN |
| | | | 055555556 | | | tidine | | protein | NRVSYIDGKQATQKTENLTPDEVSKREGINAEQIVI |
| | | | | | | triad | | | KITDQGYVTSHGDHYHYYNGKVPYDAIFSEELLM |
| | | | | | | protein | | | KDPNYKLKDEDIVNEVKGGYVIKVDGKYYVYLKD |
| | | | | | | | | | AAHADNVRTKEEINRQKQEHSQHREGGTPRNDGA |
| | | | | | | | | | VALARSQGRYTTDDGYIFNASDIIEDTGDAYIVPHG |
| | | | | | | | | | DHYHYIPKNELSASELAAAEAFLSGRGNLSNSRTY |
| | | | | | | | | | RRQNSDNTSRTNWVPSVSNPGTTNTNTSNNSNTNS |
| | | | | | | | | | QASQSNDIDSLLKQLYKLPLSQRHVESDGLVFDPA |
| | | | | | | | | | QITSRTARGVAVPHGDHYHFIPYSQMSELEERIARII |
| | | | | | | | | | PLRYRSNHWVPDSRPEQPSPQPTPEPSPGPQPAPNL |
| | | | | | | | | | KIDSNSSLVSQLVRKVGEGYVFEEKGISRYVFAKD |
| | | | | | | | | | LPSETVKNLESKLSKQESVSHTLTAKKENVAPRDQ |
| | | | | | | | | | EFYDKAYNLLTEAHKALFENKGRNSDFQALDKLL |
| | | | | | | | | | ERLNDESTNKEKLVDDLLAFLAPITHPERLGKPNSQ |
| | | | | | | | | | IEYTEDEVRIAQLADKYTTSDGYIFDEHDIISDEGDA |
| | | | | | | | | | YVTPHMGHSHWIGKDSLSDKEKVAAQAYTKEKGI |
| | | | | | | | | | LPPSPDADVKANPTGDSAAAIYNRVKGEKRIPLVR |
| | | | | | | | | | LPYMVEHTVEVKNGNLIIPHKDHYHNIKFAWFDD |
| | | | | | | | | | HTYKAPNGYTLEDLFATIKYYVEHPDERPHSNDG |
| | | | | | | | | | WGNASEHVLGKKDHSEDPNKNFKADEEPVEETPA |
| | | | | | | | | | EPEVPQVETEKVEAQLKEAEVLLAKVTDSSLKANA |
| | | | | | | | | | TETLAGLRNNLTLQIMDNNSIMAEAEKLLALLKGS |
| | | | | | | | | | NPSSVSKEKIN |
| 94 | peg.1217 | SP_2128 | 0.0625 | 8 | CHO | | | Transketolase | MILSKNREDELRKFATNIRLNTLRTLNHLGFGHYG |
| | | | | | metabolism | | | | GSLSIVEVLAVLYGEIMPMTPEIFAARDRDYFILSK |
| | | | | | | | | | GHGGPALYSTLYLNGFFDKEFLYSLNTNGTKLPSH |
| | | | | | | | | | PDRNLTPGIDMTTGSLGQGISVATGLAYGQRIRKSP |
| | | | | | | | | | FYTYAIVGDGELNEGQCWEAIQFASHQQLSNLIVF |
| | | | | | | | | | VDDNKKQLDGFTKDICNPGDFVEKFSAFGFESIRV |
| | | | | | | | | | KGSDIREIYEGIVQLKQSNNSSPKCIVLDTIKGQGV |
| | | | | | | | | | QELEEMKSNHHLRPTVEERQMLTSVVERLSQELEE |
| | | | | | | | | | TE |
| 95 | peg.1230 | SP_2142 | 0.0625 | 8 | CHO | | Yes | hydrolase, family 20 | MVRFIGLSPKQTQAIEVLKGHISLPDVEVAVTQSDQ |
| | | | | | metabolism | | | | ASISIEGEEGHYQLTYRKPHQLYRALSLLVTVLAEA |
| | | | | | | | | | DKVEIEEQAAYEDLAYMVDCSRNAVLNVASAKQ |
| | | | | | | | | | MIEVLALMGYSTFELYMEDTYQIEGQPYFGYFRGA |
| | | | | | | | | | YSAEELQEIEAYAQQFDMTFVPCIQTLAHLSAFVK |
| | | | | | | | | | WGVKEVQELRDVEDILLIGEEKVYDLIDGMFATLS |
| | | | | | | | | | KLKTRRVNIGMDEAHLVGLGRYLILNGVVDRSLL |
| | | | | | | | | | MCQHLERVLDIADKYGFHCQMWSDMFFKLMSAD |
| | | | | | | | | | GQYDRDVEIPEETRVYLDRLKDRVTLVYWDYYQD |
| | | | | | | | | | SEEKYNRNFRNHHKISHDLAFAGGAWKWIGFTPH |
| | | | | | | | | | NHFSRLVAIEANKACRANQIKEVIVTGWGDNGGET |
| | | | | | | | | | AQFSILPSLQIWAELSYRNDLDGLSAHFKTNTGLTV |
| | | | | | | | | | EDFMQIDLANLLPDLPGNLSGINPNRYVFYQDILCP |
| | | | | | | | | | ILDQHMTPEQDKPHFAQAAETLANIKEKAGNYAY |
| | | | | | | | | | LFETQAQLNAILSSKVDVGRRIRQAYQADDKESLQ |
| | | | | | | | | | QIARQELPELRSQIEDFHALFSHQWLKENKVFGLD |
| | | | | | | | | | TVDIRMGGLLQRIKRAESRIEVYLAGQLDRIDELEV |
| | | | | | | | | | EILPFTDFYADKDFAATTANQWHTIATASTIYTT |
| 96 | peg.1426 | SP_0090 | 0.0625 | 8 | transport | | | Binding-protein- | MKKFSKTLRDNWIFLLMVLPGTLWLILFFYIPVFG |
| | | | | | | | | dependent transport | NVVAFKDYHMTSNGFIDSIINSKWVGLDNFRFLFSS |
| | | | | | | | | systems, inner | RDAFIITRNTVLYNLGFIFLGLVVSVGIAIILSELRS |
| | | | | | | | | membrane | KRMVKIFQTSMLFPYFLSWVIISFFTDAFLNIDKGVF |
| | | | | | | | | component | NHLLESLGLKEVNFYADLGIWPYLLLFLGIWKGFG |
| | | | | | | | | | YSSVMYYATIMGIDPTYYEAATVDGASKWQRIRN |
| | | | | | | | | | VTIPQLTPLVTVLTILAVGNIFRADFGLFYQIPHNAG |
| | | | | | | | | | QLYNVTNVLDVYVFNGLTQTADIGMAAAAGLYQ |
| | | | | | | | | | SVVGLILVILSNLLARRVDPNSALF |
| 97 | peg.0516 | n/a | 0.066 | 5 | unknown | | | NA | MKLNKLNFLKENIRNLYSSGVIYLSLLISFIPPILLTF |
| | | | | | | | | | FILKTQGTSLGIKHISNFYAMLGMLMAVIHANRVIS |
| | | | | | | | | | RDFSHNTVSLFYNQQKNRMIYVLSNFLYAISVSIIY |
| | | | | | | | | | ALNGIVLLVIVSKLGIPGDLGLDFIVAIVVNTILLVL |
| | | | | | | | | | FYFLLSYIFYLYKLKSGLVFGILVALLLFIPNILNTM |
| | | | | | | | | | MMNTSNDLFIKAIELLPFYSLPLFVASNTMSISQYL |
| | | | | | | | | | VLITTIILLYFFTLKKSKKYSF |
| 98 | peg.0840 | SP_1780 | 0.066 | 5 | Protein | | Yes | Oligoendopeptidase f | MEQKHRSEFPEKELWDLTALYQDREDFLRAIEKAR |
| | | | | | processing | | | | EDINQFSRDYKGNLHTFEDFEKAFAELEQIYIQMSH |
| | | | | | | | | | IGNYGFMPQTTDYSNDEFANIAQAGMEFETAASVA |
| | | | | | | | | | LTFFDDALVVADEEVLDRLGELPHLTAAIRQAKIK |
| | | | | | | | | | KAHYLGADVEKALTNLGEVFYSPQDIYTKMRAGD |
| | | | | | | | | | FEMADFEAHGKTYKNSFVTYENFYQNHEDAEVRE |
| | | | | | | | | | KSFRSFSEGLRKHQNTAAAAYLAQVKSEKLLADM |
| | | | | | | | | | KGYDSVFDYLLAEQEVDRVMFDRQIDLIMKDFAP |
| | | | | | | | | | VAQRYLKHVAKVNGLEKMTFADWKLDLDSALNP |
| | | | | | | | | | EVTIDDAYDLVMKSVEPLGQEYCQEVARYQEERW |
| | | | | | | | | | VDFAANSGKDSGGYAADPYRVHPYVLMSWTGRL |
| | | | | | | | | | SDVYTLIHEIGHSGQFIFSDNHQSYFNAHMSTYYVE |
| | | | | | | | | | APSTLNELLLSDYLENQSNDPRQKRFALAHRLTDT |
| | | | | | | | | | YFHNFITHLLEAAFQRKVYTLIEEGETFGASKLNSI |
| | | | | | | | | | MKEVLTDFWGDAIEIDDDAALTWMRQAHYYMGL |
| | | | | | | | | | YSYTYSAGLVISTAGYLHLKNSGTGAEDWLNLLKS |
| | | | | | | | | | GGSKTPLESAMIIGADISTDKPLRDTIQFLSDTVDQII |
| | | | | | | | | | AYSAELGE |
| 99 | peg.1026 | SP_1937 | 0.066 | 5 | Cell wall | LytA | Yes | n-acetylmuramoyl-1- | MEINVSKLRTDLPQVGVQPYRQVHAHSTGNPHST |
| | | | | | processing | | | alanine amidase | VQNEADYHWRKDPELGFFSHIVGNGCIMQVGPVD |
| | | | | | | | | | NGAWDVGGGWNAETYAAVELIESHSTKEEFMTD |
| | | | | | | | | | YRLYIELLRNLADEAGLPKTLDTGSLAGIKTHEYCT |
| | | | | | | | | | NNQPNNHSDHVDPYPYLAKWGISREQFKHDIENG |
| | | | | | | | | | LTIETGWQKNDTGYWYVHSDGSYPKDKFEKINGT |
| | | | | | | | | | WYYFDSSGYMLADRWRKHTDGNWYWFDNSGEM |
| | | | | | | | | | ATGWKKIADKWYYFNEEGAMKTGWVKYKDTWY |
| | | | | | | | | | YLDAKEGAMVSNAFIQSADGTGWYYLKPDGTLA |
| | | | | | | | | | DKPEFTVEPDGLITVK |
| 100 | peg.1140 | SP_2052 | 0.066 | 5 | secretion | | | Competence protein | MVQEIAQEIIRSARKKGTQDIYFVPKLDAYELHMR |
| | | | | | | | | | VGDERCKIGSYDFEKFAAVISHFKFVAGMNVGEKR |
| | | | | | | | | | RSQLGSCDYAYDQKMASLRLSTVGDYRGHESLVI |
| | | | | | | | | | RLLHDEEQDLHFWFQDIEELGKQYRQRGLYLFAGP |
| | | | | | | | | | VGSGKTTLMHELSKSLFKGQQVMSIEDPVEIKQDD |
| | | | | | | | | | MLQLQLNEAIGLTYENLIKLSLRHRPDLLIIGEIRDS |
| | | | | | | | | | ETARAVVRASLTGATVFSTIHAKSIRGVYERLLELG |
| | | | | | | | | | VSEEELAVVLQGVCYQRLIGGGGIVDFASRDYQEH |
| | | | | | | | | | QAAKWNEQIDQLLKDGHITSLQAETEKISYS |
| 101 | peg.1399 | SP_0060 | 0.066 | 5 | CHO | | | beta-galactosidase | MTRFEIRDDFYLDGKSFKILSGAIHYFRVPPEDWYH |
| | | | | | metabolism | | | | SLYNLKALGFNTVETYVAWNLHEPREGEFHFEGD |
| | | | | | | | | | LDLEKFLQIAQDLGLYAIVRPSPFICAEWEFGGLPA |
| | | | | | | | | | WLLTKNMRIRSSDPAYIEAVGRYYDQLLPRLVPRL |
| | | | | | | | | | LDNGGNILMMQVENEYGSYGEDKAYLRAIRQLME |
| | | | | | | | | | ECGVTCPLFTSDGPWRATLKAGTLIEEDLFVTGNF |
| | | | | | | | | | GSKAPYNFSQMQEFFDEHGKKWPLMCMEFWDGW |
| | | | | | | | | | FNRWKEPIITRDPKELADAVREVLEQGSINLYMFH |
| | | | | | | | | | GGTNFGFMNGCSARGTLDLPQVTSYDYDALLDEE |
| | | | | | | | | | GNPTAKYLAVKKMMATHFSEYPQLEPLYKESMEL |
| | | | | | | | | | DAIPLVEKVSLFETLDSLSSPVESLYPQKMEELGQS |
| | | | | | | | | | YGYLLYRTETNWDAEEERLRIIDGRDRAQLYVDG |
| | | | | | | | | | QWVKTQYQTEIGEDIFYQGKKKGLSRLDILIENMG |
| | | | | | | | | | RVNYGHKFLADTQRKGIRTGVCKDLHFLLNWKHY |
| | | | | | | | | | PLPLDNPEKIDFSKGWTQGQPAFYAYDFTVEEPKD |
| | | | | | | | | | TYLDLSEFGKGVAFVNGQNLGRFWNVGPTLSLYIP |
| | | | | | | | | | HSYLKEGANRIIIFETEGQYKEEIHLTRKPTLKHIKG |
| | | | | | | | | | ENL |
| 102 | peg.1520 | SP_0180 | 0.066 | 5 | Bacteriocin | | | CAAX protease self- | MTVVFEFVSEMLKQFVGLDGQGLNQSNIQSTFQEQ |
| | | | | | ? | | | immunity | PLLIAVFACVIGPLVEELFFRQVLLHYLQERLSGLLS |
| | | | | | | | | | IILVGLVFALTHMHSLALSEWIGAVGYLGGGLAFSI |
| | | | | | | | | | IYVKEKENIYYPLLVHMLSNSLSLIILAISIVK |
| 103 | peg.1671 | SP_0323 | 0.066 | 5 | CHO | | | PTS System | MTMPNIIMTRIDERLIHGQGQLWVKYLGCNTVIVA |
| | | | | | transport | | | | NDEVSTDKMQQTLMKTVVPDSVAMRFFPLQKVID |
| | | | | | | | | | IIHKANPAQTIFIVVKDVKDALTLVEGGVPIKEINIG |
| | | | | | | | | | NIHNATGKEQVTRSIFLGEEDKAALKELSQTHQVT |
| | | | | | | | | | FNTKTTPTGNDGAVQVNIMDYI |
| 104 | peg.1866 | SP_0529 | 0.066 | 5 | bacteriocin | | | Transport protein | MNPNLFRSVEFYQRRYHNYATVLIIPLSLLFTFILIF |
| | | | | | | | | ComB | SLVATKEITVTSQGEIAPTSVIASIQSTSDNPILANHL |
| | | | | | | | | | VANQVVEKGDLLIKYSETMEESQKTALETQLQRLE |
| | | | | | | | | | KQKEGLGILKQSLEKATDLFSSEDEFGYHNTFMNF |
| | | | | | | | | | TKQSHDIELGISKTNTEVSNQANLTNSSSSAIEQEIT |
| | | | | | | | | | KVQQQIGEYQELRDAIVNKRARLPTGNPHQSILNR |
| | | | | | | | | | YLIASQEQTQGTAEEPFLSQINQSIAGLESSIASLKL |
| | | | | | | | | | QQAGIGSVATYDNSLATKIEVLRTQFLQTASQQQL |
| | | | | | | | | | TVENQLTELKVQLDQATQRLENNTLTAPSKGIVHL |
| | | | | | | | | | NSEFEGKNRIPTGTEIAQIFPIITDTREVLITYYVSSD |
| | | | | | | | | | YLPLLDKGQTVRLKLEKIGNHGTTIIGQLQTIDQTP |
| | | | | | | | | | TRTEQGNLFKLTALAKLSNEDSKLIQYGLQGRVTS |
| | | | | | | | | | VTAKKTYFDYFKDKILTHSD |
| 105 | peg.1929 | SP_0600 | 0.066 | 5 | transport | | | abc transporter atp- | MTLLQLQDVTYRYKNTAEAVLYQIDYNFEPGKFY |
| | | | | | | | | binding protein | SIIGESGAGKSTLLSLLAGLDSPVEGSILFQGEDIRK |
| | | | | | | | | | KGYSYHRMHHISLVFQNYNLIDYLSPLENIRLVNK |
| | | | | | | | | | KASKDTLLELGLDESQIKRNVLQLSGGQQQRVAIA |
| | | | | | | | | | RSLVSEAPVILADEPTGNLDPKTAGDIVELLKSLAQ |
| | | | | | | | | | KTGKCVIVVTHSKEVAQASDITLELKDKKLTETRN |
| | | | | | | | | | TSK |
| 106 | peg.2050 | SP_0724 | 0.066 | 5 | metabolism | | | 4-methy 1-5-beta- | MQEFTNPFPIGSSSLIHCMTNEISCEMLANGILALGC |
| | | | | | | | | hydroxy ethylthiazole | KPVMADDPREVLDFTKQSQALFINLGHLSAEKEKA |
| | | | | | | | | kinase | IRMAASYANQSSLPMVVDAVGVTTSSIRKSLVKDL |
| | | | | | | | | | LNYRPTVIKGNMSEIRSLVGLKHHGVGVDASAKD |
| | | | | | | | | | QETEDLLQVLKDWCQTYPGMSFLVTGPKDLIVSK |
| | | | | | | | | | NQVAVLENGCTELDWITGTGDLVGALTAVFLSQG |
| | | | | | | | | | KTGFEASCLAVSYLNIAAEKIVVQGMGLEEFRYQV |
| | | | | | | | | | LNQLSLLRRDENWLDTIKGEAYE |
| 107 | peg.0154 | n/a | 0. | 7 | plasmid | | | mob A MobL family | MADSFHFSVNIISRGKGKSAVASAAYISGEKIKNE |
| | | | 071428571 | | | | | protein | WDGVTHDYTRKEKILVKNIILPDHIPKEFNDRSTLW |
| | | | | | | | | | NKVEMAEKNSNAQLARQFIIGLPKELSLSENKNLV |
| | | | | | | | | | ERYIKENLTSQGMIVDYAIHDESQDKNGNIHCHIM |
| | | | | | | | | | TIMRPINEKGEFLAKSKKEYILDEKGEKVLNKNGK |
| | | | | | | | | | PKTRKVELTTWNDTGNVEQWRENFSDLCNKYLER |
| | | | | | | | | | AGAEKRVDHRSFKRQNSDYLPTIHLGSAASAMER |
| | | | | | | | | | KGIETDKGNYNREIRKYNQLVKTIKEEIKTLKGWIG |
| | | | | | | | | | NLLDNLSTAYEKFKDIERDKVIDNPKLFNLTNYLLT |
| | | | | | | | | | YSEIQKEKSKYLKGYAKTNKEKYDFKKLTSAYSYL |
| | | | | | | | | | RKNNIETIGQLQTKIETLKSNSYRLNKKAKTIHKEM |
| | | | | | | | | | EDVEKKILYYEIYKAKKEVYEEYQKKNIFTKEAFY |
| | | | | | | | | | NKHKKDIDQYKVVSGKLKKLLSDKEKLSPKKWNE |
| | | | | | | | | | EKILLM |
| 108 | peg.0244 | SP_1114 | 0. | 7 | transport | | | ABC transporter, | MIILQANKIERSFAGEVLFDNINLQVDERDRIALVG |
| | | | 071428571 | | | | | ATP-binding protein | KNGAGKSTLLKILVGEEEPTSGEINKKKDISLSYLA |
| | | | | | | | | | QDSRFESENTIYDEMLHVFDDLRRTDKQLRQMELE |
| | | | | | | | | | MGEKSGEDLDKLMSDYDRLSENFRQAGGFTYEAD |
| | | | | | | | | | IRAILNGFKFDESMWQMKIAELSGGQNTRLALAK |
| | | | | | | | | | MLLEKPNLLVLDEPTNHLDIETISWLENYLVNYSG |
| | | | | | | | | | ALIIVSHDRYFLDKVATITLDLTKHSLDRYVGNYSR |
| | | | | | | | | | FVELKEQKLATEAKNYEKQQKEIAALEDFVNRNL |
| | | | | | | | | | VRASTTKRAQSRRKQLEKMERLDKPEAGKKAAN |
| | | | | | | | | | MTFQSEKTSGNVVLTVENAAVGYDGEVLSQPINL |
| | | | | | | | | | DLRKMNAVAIVGPNGIGKSTFIKSIVDQIPFIKGEKR |
| | | | | | | | | | FGANVEVGYYDQTQSKLTPSNTVLDELWNDFKLT |
| | | | | | | | | | PEVEIRNRLGAFLFSGDDVKKSVGMLSGGEKARLL |
| | | | | | | | | | LAKFSMENNNFLILDEPTNHLDIDSKEVLENALIDF |
| | | | | | | | | | DGTLLFVSHDRYFINRVATHVLELSENGSTLYLGD |
| | | | | | | | | | YDYYVEKKATAEMSQTEEASTSNQAKEASPVNDY |
| | | | | | | | | | QAQKESQKEVRKLMRQIESLEAEIEELESQSQAISE |
| | | | | | | | | | QMLETNDADKLMELQAELDKISHRQEEAMLEWEE |
| | | | | | | | | | LSEQV |
| 109 | peg.0298 | SP_1185 | 0. | 7 | transport | | | Pts system | MNKLIAFIEKGKPFFEKLSRNIYLRAIRDGFIAGMP |
| | | | 071428571 | | | | | | VILFSSIFILIAFVPNSWGFKWSDEVVAFLMKPYSYS |
| | | | | | | | | | MGILALLVAGTTAKSLTDSVNRSMEKTNQINYMST |
| | | | | | | | | | LLAAIVGLLMLAADPIESGLATGFLGTKGLLSAFLA |
| | | | | | | | | | AFVTVAIYKVCVKNNVTIRMPDEVPPNISQVFKDVI |
| | | | | | | | | | PFTLSVVSLYALDLLARHFVGASVAESIGKFFAPLF |
| | | | | | | | | | SAADGYLGITIIFGAFAFFWFVGIHGPSIVEPAIAAIT |
| | | | | | | | | | YANAEVNLNLLQQGMHADKILTSGTQMFIVTMGG |
| | | | | | | | | | TGATLVVPFMFMWLTKSKRNRAIGRASVVPTFFG |
| | | | | | | | | | VNEPILFGAPLVLNPIFFIPFIFVPIANVWIFKFFIE |
| | | | | | | | | | TLGMNSFTANLPWTTPAPLGLVLGTNFQVLSFILAAL |
| | | | | | | | | | LIVVDVVIYYPFLKVYDEQILEEERSGKSNDELKEK |
| | | | | | | | | | VAANFNTAKADAILEKAGVEAAQNTITKETNVLVL |
| | | | | | | | | | CAGGGTSGLLANALNKAAAEYNVPVKAAAGGYG |
| | | | | | | | | | AHREMLPEFDLVILAPQVASNFEDMKAETDKLGIK |
| | | | | | | | | | LAKTEGAQYIKLTRDGKGALAFVQAQFD |
| 110 | peg.0427 | n/a | 0. | 7 | unknown | | | NA | LQNLKKYLLLDSGLVVGYTLENAQLKLNKLKKDT |
| | | | 071428571 | | | | | | ENNPLFGEEYFAENPKLWEVRFDNSYPNVIYDKEL |
| | | | | | | | | | KLYRCWYQTFVSDEASEETPLAERGEKEYIVKSSR |
| | | | | | | | | | MTALCYAESKDGVKWEKPNLNLVKFKGSKDNNIV |
| | | | | | | | | | LMHAHGAGIMLDEQELDPKKRYKLVSKLEFSPNL |
| | | | | | | | | | HYMVVVFSEDGIHFDEPIPWKKWNPAADSHNFPFR |
| | | | | | | | | | DPKTGKYAVITRTWANNVRLSAITFSDDFINWSEP |
| | | | | | | | | | KEILRGDGFEDQIYSMPVISYADIYLGFASMYHEGD |
| | | | | | | | | | TLNENYDKVDLELKFSANLEQWDSVCKGDYLIER |
| | | | | | | | | | GGGVYPQNADFDASCIFTAAPIIEDDKIWFYYMGG |
| | | | | | | | | | NGNHTGFRESSLARGYIKKDQFAYYTNKREGIPAK |
| | | | | | | | | | ISTVSFQTYDDEIYILADIPENGYVKVAVGTKYGNA |
| | | | | | | | | | YSDFGQEDISLVQVSDGKYKLEFNKKSILDLGNSP |
| | | | | | | | | | VAFKFEIENAKLYGIEGSIQQHYLKY |
| 111 | peg.0439 | SP_1344 | 0. | 7 | bacteriocin | | | serine threonine | MNNDEWVYQYPIGKFVERQGWKIHISSEYNSSHEL |
| | | | 071428571 | | | | | protein kinase | LQDVAKICHEMRIPFKHLSTEDKFIMRNGKLVSRG |
| | | | | | | | | | FSGKFITCYPNQNELESVLQRLESALKQYNGPYILS |
| | | | | | | | | | DKRWDEAPIYLRYGVFRPSRDDEKKVVIDELIVGD |
| | | | | | | | | | EVVKDERLPVFKIPKGIVPPDFLNKWLDKKDKKQG |
| | | | | | | | | | DFPFIIDNAIRFSNSGGIYNARLKEDGKKIILKEARP |
| | | | | | | | | | YTGLGFDGTYSSERLASECKALKILNEWSEMPKIY |
| | | | | | | | | | WYGKIWEHTFLGIEHMKGVPLNRWVTNNFPLYEV |
| | | | | | | | | | VDKTKDYLLRVSKIVEKLIDLTNKFHSENVYHQDL |
| | | | | | | | | | HLGNILVKDEDEISIIDWEQAVFSNDEKVVHKVAA |
| | | | | | | | | | PGFRAWRETLPSEIDWYGIRQIAHYLYMPLVTTSD |
| | | | | | | | | | LTYNYVSQTRIEGKKLFESLGYTREHIDYVESLLSY |
| | | | | | | | | | LDSKCPQIENISRKKVLKPMHEIRTIESEQDIQDFIIK |
| | | | | | | | | | AFLLVN |
| 112 | peg.0575 | SP_1493 | 0. | 7 | adhesin | | | surface protein | MNIILIAKLLRENTNTKANALNNGWARSGSEEFKK |
| | | | 071428571 | | | | | | FSHFVGVDKGIVRTNVLTGKKLSDKIRKEVGSGDS |
| | | | | | | | | | KLGKGGYFSTGDVLLGKDVVSYTVQVFSENNERV |
| | | | | | | | | | GVNTQSHRVQYNLPILADFSVIQDTVEPSRTVVEKI |
| | | | | | | | | | IPKLNIPEEEKGKITEEIKKKKKTSELAELISENVKV |
| | | | | | | | | | RYVDEQGRLLSLKNDTGIGEKESDGTYITNKKQLI |
| | | | | | | | | | GTSYNVTDKKLSSMTTTDGKYYTFKEVDTNSASL |
| | | | | | | | | | TGNIVSEGRTVTLVYRESEAPTTVTVTANYYKEGS |
| | | | | | | | | | QEKLAESVIKADLAIGSEYTTESKTIEGKTTTEDKE |
| | | | | | | | | | NRVITRKTTYTLVATPANAYQKTVQQLTITTVRML |
| | | | | | | | | | RKQWFPKQQPLLRRRL |
| 113 | peg.0613 | SP_1527 | 0. | 7 | Peptide | AliB | | Oligopeptide-binding | MKKSKSKYLTLAGLVLGTGVLLSACGNSSTASKT |
| | | | 071428571 | | transport | | | protein | YNYVYSSDPSSLNYLAENRAATSDIVANLVDGLLE |
| | | | | | | | | | NDQYGNIIPSLAEDWTVSQDGLTYTYKLRKDAKW |
| | | | | | | | | | FTSEGEEYAPVTAQDFVTGLQYAADKKSEALYLV |
| | | | | | | | | | QDSVAGLDDYITGKTSDFSTVGVKALDDQTVQYT |
| | | | | | | | | | LVKPELYWNSKTLATILFPVNADFLKSKGDDFGKA |
| | | | | | | | | | DPSSILYNGPFLMKALVSKSAIEYKKNPNYWDAKN |
| | | | | | | | | | VFVDDVKLTYYDGSDQESLERNFTAGAYTTARLFP |
| | | | | | | | | | NSSSYEGIKEKYKNNIIYSMQNSTSYFFNFNLDRKS |
| | | | | | | | | | YNYTSKTSDIEKKSTQEAVLNKNFRQAINFAFDRTS |
| | | | | | | | | | YGAQSEGKEGATKILRNLVVPPNFVSIKGKDFGEV |
| | | | | | | | | | VASKMVNYGKEWQGINFADGQDPYYNPEKAKAK |
| | | | | | | | | | FAEAKKELEAKGVQFPIHLDKTVEVTDKVGIQGVS |
| | | | | | | | | | SIKQSIESVLGSDNVVIDIQQLTSDEFDSSGYFAQTA |
| | | | | | | | | | AQKDYDLYHGGWGPDYQDPSTYLDIFNTNSGGVL |
| | | | | | | | | | QNLGLEPGEANDKAKAVGLDVYTQMLEEANKEQ |
| | | | | | | | | | DPAKRYEKYADIQAWLIDSSLVLPSVSRGGTPSLR |
| | | | | | | | | | RTVPFAAAYGLTGTKGVESYKYLKVQDKIVTTDE |
| | | | | | | | | | YAKAREKWLKEKEESNKKAQEELAKHVK |
| 114 | peg.0695 | SP_1612 | 0. | 7 | regulation | | | serine threonine | MDLLEKECLKCDKNFQQGDIWNYYYLSDKMPAQ |
| | | | 071428571 | | | | | protein kinase | GWKIHISSQIKDAVNIFKIVYKLSQLNNCSFKVVKN |
| | | | | | | | | | LEELKKINSPREMSPTANKFITLYPKSESKAKSMIC |
| | | | | | | | | | NLTNRLSEFKAPKILSDYQCGMHSPVHYRYGAFLK |
| | | | | | | | | | KQAYDEKNKKVIYLLLDEKRKNYVEDKRQNFPSL |
| | | | | | | | | | PSWKMDLFSEEEKRIYFQTTCEVSSKDSAINKYKM |
| | | | | | | | | | EKIIKRSNKGNVYRAIRKSDGQKVIIKQSRPFVNYD |
| | | | | | | | | | AEGEWTALDDIKNEAHMLKKLADKSYTTNLTDEF |
| | | | | | | | | | YIVDDYFLVQEQVDGLNFEEFIRETEHSLNIREKTL |
| | | | | | | | | | DNIVNIVSYIHKLGI |
| 115 | peg.0853 | SP_1790 | 0. | 7 | recombinat | | Yes | recombination factor | MPDNLALRMRPKTIDQVIGQEHLVGPGKIIRRMVE |
| | | | 071428571 | | ion | | | protein RarA | ANRLSSMILYGPPGIGKTSIASAIAGTTKYAFRTFN |
| | | | | | | | | | ATVDSKKRLQEISEEAKFSGGLVLLLDEIHRLDKTK |
| | | | | | | | | | QDFLLPLLESGLVIMIGATTENPFFSVTPAIRSRVQIF |
| | | | | | | | | | ELEPLSNQDVKEALQIALSNPERGFDFPIELDEDAL |
| | | | | | | | | | DFIATSTNGDLRSAFNSLDLAVLSTPENDEGIRHITL |
| | | | | | | | | | DIMENSLQRSYITMDKDGDGHYDVLSALQKSIRGS |
| | | | | | | | | | DVDASLHYTARLIEAGDLPSLARRLTVIAYEDIGLA |
| | | | | | | | | | NPEAQIHTVTALDAAQKIGFPEARILIANVVIDLALS |
| | | | | | | | | | PKSNSAYVAMDKALADLKTSGHLPIPRHLRDGHY |
| | | | | | | | | | SGSKELGNAQDYLYPHNYPGNWVKQDYLPEKIRN |
| | | | | | | | | | HHYFQAEDTGKYERALAQRKEAIDRLRKI |
| 116 | peg.0859 | SP_1800 | 0. | 7 | regulation | | | M protein trans- | MRDLLSKKSHRQLELLELLFEHKRWFHRSELAELL |
| | | | 071428571 | | | | | acting positive | NCTERAVKDDLSHVKSAFPDLIFHSSTNGIRIINTDD |
| | | | | | | | | regulator (MGA) | SDIEMVYHHFFKHSTHFSILEFIFFNEGCQAESICKE |
| | | | | | | | | PRD domain | FYISSSSLYRIISQINKVIKRQFQFEVSLTPVQIIGNE |
| | | | | | | | | | RDIRYFFAQYFSEKYYFLEWPFENFSSEPLSQLLELV |
| | | | | | | | | | YKETSFPMNLSTHRMLKLLLVTNLYRIKFGHFMEV |
| | | | | | | | | | DKDSFNDQSLDFLMQAEGIEGVAQSFESEYNISLDE |
| | | | | | | | | | EVVCQLFVSYFQKMFFIDESLFMKCVKKDSYVEKS |
| | | | | | | | | | YHLLSDFIDQISVKYQIEMENKDNLIWHLHNTAHL |
| | | | | | | | | | YRQELFTEFILFDQKGNTIRNFQNIFPKFVSDIKKEL |
| | | | | | | | | | SHYLETLEVCSSSMMVNHLSYTFITHTKHLVINLLQ |
| | | | | | | | | | NQPKLKVLVMSNFDQYHAKFVAETLSYYCSNNFE |
| | | | | | | | | | LEVWTELELSKESLEDSPYDIIISNFIIPPIENKRLI |
| | | | | | | | | | YSNNINTVSLIYLLNAMMFIRLDE |
| 117 | peg.0937 | SP_1872 | 0. | 7 | transport | | | Periplasmic binding | MKTSLKLYFTALVASFLLLLGACSTNSSTSQTETSS |
| | | | 071428571 | | | | | protein | SAPTEITIKSSLDEVKLSKVPEKIVTFDLGAADTIRA |
| | | | | | | | | | LGFEKNIVGMPTKTVPTYLKDLAGTVKNVGSMKE |
| | | | | | | | | | PDLEAIAALEPDLIIASPRTQKFVDKFKEIAPTVLFQ |
| | | | | | | | | | ASKDDYWTSTKANIESLASAFGETGTQKAKEELAK |
| | | | | | | | | | LDKSIQEVATKNESSDKKALAILLNEGKMAAFGAK |
| | | | | | | | | | SRFSFLYQTLKFKPTDTTFEDSRHGQEVSFESVKEI |
| | | | | | | | | | NPDILFVINRTLAIGGDNSSNDGVLENALIAETPAA |
| | | | | | | | | | KNGKIIQLTPDLWYLSGGGLESTKLMIEDIQKALK |
| 118 | peg.1088 | SP_2000 | 0. | 7 | regulation | | | response regulator | MKVLVAEDQSMLRDAMCQLLAFQADVESVLQAK |
| | | | 071428571 | | | | | | NGQEAIQLLEKESVDIAILDVEMPVKTGLEVLEWIR |
| | | | | | | | | | AEKLETKVVVVTTFKRPGYFERAVKAGVDAYVLK |
| | | | | | | | | | ERNIADLMQTLHTVLEGGKEYSPELMEVVMMHPN |
| | | | | | | | | | PLTEQEIAVLKGIAQGLSNQEIADQLYLSNGTVRNY |
| | | | | | | | | | VTNILSKLDAGNRTEAANIAKESGWL |
| 119 | peg.1089 | SP_2001 | 0. | 7 | regulation | | Yes | Histidine kinase | MLERLKSIHYMFWISLIFMIFPILPVVTGWLSAWHL |
| | | | 071428571 | | | | | | LIDILFVVAYLGVLITKSQRLSWLYWGLMLSYVVG |
| | | | | | | | | | NTAFVAVNYIWFFFFLSNLLSYHFGVRSFNSLHVR |
| | | | | | | | | | TFLLAQVLVVGQLLIFQEVEVEFLVYLLGILTFIDL |
| | | | | | | | | | MTFGLVRIRIMEDLKEAQAKQNAQINLLLAENERS |
| | | | | | | | | | RIGQDLHDSLGHTFAMLSVKTDLALQLFQIQAYPQ |
| | | | | | | | | | VEKELREIQQISKESMREVRTIVENLKSRTLISELET |
| | | | | | | | | | VKKMLEIAGIEVETDNQLDTASLTQKLESTASMILL |
| | | | | | | | | | ELVTNIIKHAKASKVYLKLERTEKELILTVRDDGCG |
| | | | | | | | | | FASIKGDDLHTVRDRILPFSGEVKVISWKQPTEVQV |
| | | | | | | | | | RLPYKERK |
| 120 | peg.1119 | SP_2032 | 0. | 7 | regulation | | | PRD domain protein | MVLDKASCDLLQYLMDQETSKTIMAISKDLKESRR |
| | | | 071428571 | | | | | | KIYYHIDKINAALGDEALHIISIPRIGIHLTEEQRDAC |
| | | | | | | | | | CKLLSEVDSYDYIMSAHERMMIMLLWIGISKERITI |
| | | | | | | | | | EKLIELTEVSRNTVLNDLNSIRYQLTLEQYQVTLQV |
| | | | | | | | | | SKSQGYHLHAHPLNKIQYLQSLLYHIFMEENATFV |
| | | | | | | | | | SILEDKMKERLDDECLLSVEMNQFFKEQVPLVEQD |
| | | | | | | | | | LGKKINHHEITFMLQVLPYLLLSCHNVEQYQERHQ |
| | | | | | | | | | DIEKEFSLIRKRIEYQVSKKLGERLFQKFEISLSGLE |
| | | | | | | | | | VSLVAVLLLSYRKDLDIHAESDDFRQLKLALEEFI |
| | | | | | | | | | WYFESQIRMEIENKDDLLRNLMIHCKALLFRKTYG |
| | | | | | | | | | IFSKNPLTKQIRSKYGELFLATRKSAEILEGAWFIRL |
| | | | | | | | | | TDDDIAYLTIHIGGFLKYTPSSQKNMKKVYLVCDE |
| | | | | | | | | | GVAVSRLLLKQCKLYFPNEQIDTVFTTEQFKSVEDI |
| | | | | | | | | | AQVDVVITTNDDLDSRFPILRVNPILEAEDILKMLD |
| | | | | | | | | | YLKHNIFRNKSKSFSENLSSLISSYIVDSKLASKFQE |
| | | | | | | | | | EVQTLINQEIVVQAFLEDI |
| 121 | peg.1192 | SP_2106 | 0. | 7 | CHO | | Yes | Phosphorylase is an | MLSLQEFVQNRYNKTIAECSNEELYLALLNYSKLA |
| | | | 071428571 | | metabolism | | | important allosteric | SSQKPVNTGKKKVYYISAEFLIGKLLSNNLINLGLY |
| | | | | | | | | enzyme in | DDVKKELAAAGKDLIEVEEVELEPSLGNGGLGRLA |
| | | | | | | | | carbohydrate | ACFIDSIATLGLNGDGVGLNYHFGLFQQVLKNNQQ |
| | | | | | | | | metabolism. | ETIPNAWLTEQNWLVRSSRSYQVPFADFTLTSTLY |
| | | | | | | | | Enzymes from | DIDVTGYETATKNRLRLFDLDSVDSSIIKDGINFDK |
| | | | | | | | | different sources | TDIARNLTLFLYPDDSDRQGELLRIFQQYFMVSNG |
| | | | | | | | | differ in their | AQLIIDEAIEKGSNLHDLADYAVVQINDTHPSMVIP |
| | | | | | | | | regulatory | ELIRLLTARGIELDEAISIVRSMTAYTNHTILAEALE |
| | | | | | | | | mechanisms and in | KWPLEFLQEVVPHLVPIIEELDRRVKAEYKDPAVQI |
| | | | | | | | | their natural | IDESGRVHMAHMDIHYGYSVNGVAALHTEILKNS |
| | | | | | | | | substrates. However, | ELKAFYDLYPEKFNNKTNGITFRRWLMHANPRLS |
| | | | | | | | | all known | HYLDEILGDGWHHEADELEKLLSYEDKAAVKEKL |
| | | | | | | | | phosphorylases share | ESIKAHNKRKLARHLKEHQGVEINPNSIFDIQIKRL |
| | | | | | | | | catalytic and | HEYKRQQMNALYVIHKYLDIKAGNIPARPITIFFGG |
| | | | | | | | | structural properties | KAAPAYTIAQDIIHLILCMSEVIANDPAVAPHLQVV |
| | | | | | | | | (By similarity) | MVENYNVTAASFLIPACDISEQISLASKEASGTGNM |
| | | | | | | | | | KFMLNGALTLGTMDGANVEIAELVGEENIYIFGED |
| | | | | | | | | | SETVIDLYAKAAYKSSEFYAREAIKPLVDFIVSDAV |
| | | | | | | | | | LAAGNKERLERLYNELINKDWFMTLLDLEDYIKV |
| | | | | | | | | | KEQMLADYEDRDAWLDKVIVNISKAGFFSSDRTIA |
| | | | | | | | | | QYNEDIWHLN |
| 122 | peg.1485 | SP_0145 | 0. | 7 | transport | | | Major Facilitator | MKVFLQNRDFRQLTINQWISTLGDTIFYLAFLNYV |
| | | | 071428571 | | | | | | ADASFAPLTILLITISETLPQILQIFLGVLADFQHHRV |
| | | | | | | | | | LKYTVISFAKFLLYSIVSLSLSGQSFSLLLVAFICLLN |
| | | | | | | | | | LLSDTLSYFSGAMLTPIFIRIIGQDHLAEAIGFKQST |
| | | | | | | | | | VSLVKTISNILGGVLLGILSIQFISLLNALTFLIAFLG |
| | | | | | | | | | ILFIKTDLLKVEKTISYQEGLSVKSFCQHLLQSSKLI |
| | | | | | | | | | WNMNKVLLVLFIISTSQAVINVTVPVSTLFLRNQPF |
| | | | | | | | | | LNLQTGQSLALLSTFELSALIVGSLVSGYLQHTISIK |
| | | | | | | | | | TALYASLVIQLLLLVGFATVRFDWILIFSTLDAFFA |
| | | | | | | | | | GVLSPRLQELVFKQIPEESMGAVQSSIGAITVVLPSL |
| | | | | | | | | | FTIALVTIATSFGTLAVSFVLLLFLLVAFVMLLNIRE |
| | | | | | | | | | SI |
| 123 | peg.1514 | SP_0176 | 0. | 7 | CHO | | | Catalyzes the | MEYRKIQEALEALQKGRLVLVIDDKDRENEGDLIC |
| | | | 071428571 | | metabolism | | | conversion of D- | SAQAATTENVNFMATYAKGLICMPMSESLANQLM |
| | | | | | | | | ribulose 5-phosphate | LSPMVENNTDNHKTAFTVSIDYKETTTGISAEERGL |
| | | | | | | | | to formate and 3,4- | TARMCVAEDITPSDFRRPGHMFPLIAKKGGVLERS |
| | | | | | | | | dihydroxy-2- | GHTEATVDLLKLAGLKECGLCCEIMNHDGKMMR |
| | | | | | | | | butanone 4-phosphate | TDDLIQFSKKHNIPLITIKELQEYRKVYDQLVERVS |
| | | | | | | | | (By similarity) | TVNMPTRYGNFKAISYIDKLNGEHHLALIMGNIED |
| | | | | | | | | | EANVLCRVHSECLTGDVLGSLRCDCGQQFDKAMK |
| | | | | | | | | | MIVENGSGVLLYLRQEGRGIGLINKLKAYHLQDQG |
| | | | | | | | | | MDTLDANLALGFEGDLREYHIGAQMLKDLGLQSL |
| | | | | | | | | | HLLTNNPDKVEQLEKYGITISSRISIEIEANPYDSFYL |
| | | | | | | | | | ETKKNRMGHILNMEEK |
| 124 | peg.1632 | n/a | 0. | 7 | CHO | | | glycoside hydrolase | MSKFPKEFLWGGATAANQYEGAYNVGDKGLSVQ |
| | | | 071428571 | | metabolism | | | family 1 | DVTPRGGVPVSDNDPNPFITELPTEDNLKLEGIDFY |
| | | | | | | | | | HRYKEDVALFAEMGFKVFRTSIAWSRIFPNGDELE |
| | | | | | | | | | PNEEGLQFYDNLFDELAKYGIEPLVTLSHYETPLHL |
| | | | | | | | | | ARKYNGWVNRDLIGFYERYVRTVFNRYKNKVKY |
| | | | | | | | | | WLTFNEINSVLHVPFISGGIATPVEKLSKQDLYQAV |
| | | | | | | | | | HHELVASALATKVGHEINPEFKIGCMVLAMPTYP |
| | | | | | | | | | MTPKPEDVLAAREFENQNYLFSDIHARGKYPKYIQ |
| | | | | | | | | | RFFKENDINIKFESGDKELLAENTVDFISFSYYMSS |
| | | | | | | | | | VQAHDPENYQSGIGNLLGGIANPYLESSEWGWQV |
| | | | | | | | | | DPIGLRIVLNNLYDRYQLPLFIVENGLGAKDVLIEG |
| | | | | | | | | | ADGPTVDDDYRIDYLKKHLQQVGEAIEDGVELLG |
| | | | | | | | | | YTTWGCIDLVSASTAQMSKRYGFIYVDRNDDGSG |
| | | | | | | | | | TLNRYKKKSFYWYKEVIESNGETLYD |
| 125 | peg.2032 | SP_0706 | 0. | 7 | unknown | | | NA | LEMGKLSSHMWRLNQIIYTKYFWGYVLFWILICLG |
| | | | 071428571 | | | | | | LWYWLEGNDRLVIEILKGPNLSQNSFLVLSIWLLH |
| | | | | | | | | | WFIIHTFFLAVVYRRRASDFFMEVIRFSSIKLWIRYQ |
| | | | | | | | | | IWTCFLYGLILIMVKVLVIQFMLQLPNWDIGVLFIV |
| | | | | | | | | | DSLNACVLSLLCFMLYALGANVQMNFACVSFFLL |
| | | | | | | | | | MIVFGGLFVGNRTNYLFYILNRGNGDIGRDLFLQL |
| | | | | | | | | | LFLVFLFKSIFYFTRQKRRFIE |
| 126 | peg.0083 | SP_0965 | 0.083 | 6 | Cell wall | | | endo-beta-N- | MKKVRFIFLALLFFLASPEGAMASDGTWQGKQYL |
| | | | | | processing | | | acetyl- | KEDGSQAANEWVFDTHYQSWFYIKADANYAENE |
| | | | | | | | | eglucosaminidas | WLKQGDDYFYLKSGGYMAKSEWVEDKGSFYYLD |
| | | | | | | | | | QDGKMKRNAWVGTSYVGATGAKVIEDWVYDSQ |
| | | | | | | | | | YDAWFYIKADGQHAEKEWLQIKGKDYYFKSGGY |
| | | | | | | | | | LLTSQWINQAYVNASGAKVQQGWLFDKQYQSWF |
| | | | | | | | | | YIKENGNYADKEWIFENGHYYYLKSGGYMAANE |
| | | | | | | | | | WIWDKESWFYLKFDGKIAEKEWVYDSHSQAWYY |
| | | | | | | | | | FKSGGYMTANEWIWDKESWFYLKSDGKIAEKEW |
| | | | | | | | | | VYDSHSQAWYYFKSGGYMTANEWIWDKESWFYL |
| | | | | | | | | | KSDGKIAEKEWVYDSHSQAWYYFKSGGYMTANE |
| | | | | | | | | | WIWDKESWFYLKSDGKMAEKEWVYDSHSQAWY |
| | | | | | | | | | YFKSGGYMAKNETVDGYQLGSDGKWLGGKTTNE |
| | | | | | | | | | NAAYYQVVPVTANVYDSDGEKLSYISQGSVVWLD |
| | | | | | | | | | KDRKSDDKRLAITISGLSGYMKTEDLQALDASKDF |
| | | | | | | | | | IPYYESDGHRFYHYVAQNASIPVASHLSDMEVGKK |
| | | | | | | | | | YYSADGLHFDGFKLENPFLFKDLTEATNYSAEELD |
| | | | | | | | | | KVFSLLNINNSLLENKGATFKEAEEHYHINALYLLA |
| | | | | | | | | | HSALESNWGRSKIAKDKNNFFGITAYDTTPYLSAK |
| | | | | | | | | | TFDDVDKGILGATKWIKENYIDRGRTFLGNKASG |
| | | | | | | | | | MNVEYASDPYWGEKIASVMMKINEKLGGKD |
| 127 | peg.0162 | SP_1043 | 0.083 | 6 | unknown | | Yes | NA | MVVYIRQSKLPSEVSINKYHAQVGAYLQGEEAVL |
| | | | | | | | | | YQSFSEIKELTSEDIVVDYIMETRALLKMMGLNVP |
| | | | | | | | | | VHDYPIELKEFYGRKIYAGILGEIVNIPDNWGKFIKP |
| | | | | | | | | | KAGSKVFTGRVVNGTHDLIGIGLPFDYPIWISEVVE |
| | | | | | | | | | FIAEWRCFVLDGRVLDVRPYTGDYHAQFDASVIDE |
| | | | | | | | | | AISCWKDAPIAYGLDIGVTRDGRTLVVEVNDGYAL |
| | | | | | | | | | GNYGLSPLKSINFHRARWKEMVKPYFEKNEIFKIQ |
| | | | | | | | | | QDVVF |
| 128 | peg.0171 | SP_1052 | 0.083 | 6 | unknown | | | atpase involved in | MLAEAIIKNGIEIVVVTDHNTTKGIKKLQMAVSIIM |
| | | and | | | | | | dna repair | KNYPIYDIHPHILHGVEISAADKLHIVCIYDYEQES |
| | | SP_1053 | | | | | | | WVNQWLSENIISEKDGSYQHSLTIMKDFNNQKIVN |
| | | | | | | | | | YIAHFNSYDILKKGSHLSGAYKRKIFSKENTRFLEF |
| | | | | | | | | | NINSKESSQQLDILYKEVGVLSLGQKVVAMLDFLL |
| | | | | | | | | | AYSDYSKDFRPLIIDQPEDNLDNRYIYRHLVQQFRD |
| | | | | | | | | | VKAQRQIILATHNATIVTNSMTDQVVIMESDGVNG |
| | | | | | | | | | WIESQGYVSEKYIKNHIINQLEGGKDSFKHKMSIYE |
| | | | | | | | | | TALSE |
| 129 | peg.0283 | SP_1170 | 0.083 | 6 | unknown | | Yes | NA | MSEVDFNEAVNYEFTSDTCQLANSIYQSLFKFFDK |
| | | | | | | | | | KNFSGDLIFTWKSPSLVKEGDYIGRRDSQVDNLRVI |
| | | | | | | | | | GNIFPNYLTNRKYSLNMNRNGCMGDFPHDFFDIYL |
| | | | | | | | | | DHVAKYAYEQKVNNIKEYYPLKRAILHQENALYF |
| | | | | | | | | | RFFSNFDDFLEKNYLKTIWQVSKETPFSEMDFNMF |
| | | | | | | | | | KNISEKIIFERGSKMLNDLKSNYKK |
| 130 | peg.0548 | n/a | 0.083 | 6 | transport | | | abc transporter, ATP- | MVNNVAVKVSNLSKEFLLGQDKTVSILKDISLSVN |
| | | | | | | | | binding protein | YGEFVSILGVSGSGKSTLLSCLSSLSEPTSGEVVING |
| | | | | | | | | | VNPYTLKEGKLAKFRRQDIAIIFQNYNLVPALPVLE |
| | | | | | | | | | NVTLPLRLSGKSVDSNKVKKMLDSLNFKAELSSLV |
| | | | | | | | | | ATLSGGEQQKVAITRAIIADSKIIFADEPTGALDSVS |
| | | | | | | | | | RKLIFETLRNLASQGKCVLMVTHDIELASKTDRALI |
| | | | | | | | | | LKDGKISRQIIKPSADELYQALESSKD |
| 131 | peg.0607 | SP_1523 | 0.083 | 6 | regulation | | | SNF2 family | MAKLIPGKIRIEGVALYETGKVDIIKEKNNRLYARV |
| | | | | | | | | | AEEELRYSLEDDLVFCACDFFQKRGYCVHLAALE |
| | | | | | | | | | HFLKNDERGQEILWSLEEGHEEKEAVETKVTLGGK |
| | | | | | | | | | FLDRILSPKSECAYELSAVGQVEAGTNHILWTLRIG |
| | | | | | | | | | QINSQKYYVIRDIPLFLRIVEQRKSYMIGKIYEESLS |
| | | | | | | | | | WEAFDDASQELLTFLHGLTEEGLVSDLFFQNQGRH |
| | | | | | | | | | LFFPLTFFEQGVNLLMALPHFQFDHQVDSYQTLLF |
| | | | | | | | | | QDMHADANLFAFTVTEYSDYFEMEISESPRVNVFY |
| | | | | | | | | | QGAVLFHKGQVYFLTDQQMRLLKEIKALPVGQQG |
| | | | | | | | | | KKYLQFDSSDRDKLASCLTLFIQMGTVSAPERLQIK |
| | | | | | | | | | TFAPSFYFDREEDNRIRLEIQFDYGNRQVSSRQELE |
| | | | | | | | | | ELPFSSDADLEERIFQVCLAAGFEADFQSWRQALK |
| | | | | | | | | | AESVYHFFHDIIPVFEKLGHVDLSDKLEELYSLASP |
| | | | | | | | | | QVQIASKGGLLEIQFDFQDIAQEEIDQAMQALVAN |
| | | | | | | | | | QDFYIDSSNQVYFFDEETKKIRQNLQELGQFELKD |
| | | | | | | | | | GTLQARKSLAYSLAHLFEGRDRVSFSQEFQNLAQD |
| | | | | | | | | | LTHPEDFPRQATQVQADLRDYQEKGIGWLQMLHH |
| | | | | | | | | | YGFGGILADDMGLGKTLQTIAFLTSQVTKESRVLIL |
| | | | | | | | | | APSGLIYNWADEFQKFAPQLDVAVVHGLKASREEI |
| | | | | | | | | | LAESHQIYVTSYATFRQDSELYQGMAFDFLFLDEA |
| | | | | | | | | | QVMKNAQTKIAQTLRQFVVPSVFALSGTPIENHLG |
| | | | | | | | | | ELWSIFQIVMPGLLPSKKEFMKLPAERVAQFIKPFV |
| | | | | | | | | | MRRKKEEVLTELPDLIEVVYKNELEDQQKAIYLAQ |
| | | | | | | | | | LQQMRDHLAQVSEQEFQRSRVEILSGLMRLRQICD |
| | | | | | | | | | TPALFMEDYQGASGKLDSLRDLLVQVADGGHRVL |
| | | | | | | | | | IFSQFKGMLEKIEQELPDLGLTSFKITGSTPAKERQD |
| | | | | | | | | | MTKAFNQGERDAFLISLKAGGVGLNLTGADTVILV |
| | | | | | | | | | DLWWNPAVEAQAIGRAHRMGQEETVEVYRLVTK |
| | | | | | | | | | GTIEEKIQELQEQKKHLVSQVLDGTESRGSLTLAEI |
| | | | | | | | | | REILGISEAST |
| 132 | peg.0611 | SP_1526 | 0.083 | 6 | transport | | | ABC transporter | LTEKIQEHELIKTNQAEKSVQDVLDNCIERVQNNSL |
| | | | | | | | | transmembrane | KSDRVTSFETPFALLFIFATIAVMLTYGGYRVSAGY |
| | | | | | | | | region | ISVGTLVSFLIYLFQLLNPISNIANFVTVYSRSKGSS |
| | | | | | | | | | VALDNLLAVPKEKFEGGKSVSGQGLNFNHVYFGY |
| | | | | | | | | | DENRPVLKDITCSIFKGQKLLLLDHLDQENQRLCV |
| | | | | | | | | | C |
| 133 | peg.0696 | n/a | 0.083 | 6 | bacteriocin | | | serine threonine | MTPIKDKVRRVKTPMMVNPDTDLTISSVQQDYFSL |
| | | | | | | | | protein kinase | ALIGFSLLTGDFLSFSKGDQKTGLSAFIKICHLIKIAR |
| | | | | | | | | | LDNKITKQQEYWLYDLLMMSQGEKIQKIKQLKQV |
| | | | | | | | | | TSDILLNTPDFSSYFEKYNFKEEAENIKSYLLAKSM |
| | | | | | | | | | DKSGRLFPSNEFGEFVSPVSFQHGFGGVLFFMNKY |
| | | | | | | | | | YVEEDENTVKEWLTKLENYEAANFLHGYSLLFGK |
| | | | | | | | | | AGFLFGILDRYEKTKERYLIDISKRLVDHLMRVYD |
| | | | | | | | | | NISNLDFALGKSGILLSLMKYCTIFDDKKLANFIKN |
| | | | | | | | | | NINDAYSLLESEDNGDIYSNNFAHGRSGAAYVLKA |
| | | | | | | | | | YTDIFGDSRYQNHLQKFSDGISELLEEKLSSFSKLD |
| | | | | | | | | | NLGLSWCDGVSGLILYLCLIDKERYSEIIYKSQLEM |
| | | | | | | | | | VQQYEAMGTSFCHGLSSLLQTTIYNKNQKVEQFIK |
| | | | | | | | | | KNLIDSFLS |
| 134 | peg.0762 | SP_1688 | 0.083 | 6 | CHO | | | ABC transporter, | MKKKSGIYLDILSHVLLVGATIVAVFPLVWIIISSVK |
| | | | | | transport | | | permease | GKGELTQYPTRFWPEQFTLDYFTHVINDLHFIDNIR |
| | | | | | | | | | NSLIIALATTLIAIIISAMAAYGIVRFFPKLGAIMSRL |
| | | | | | | | | | LVITYIFPPILLAIPYSIAIAKVGLTNSLFGLMMVYLS |
| | | | | | | | | | FSVPYAVWLLVGFFQTVPIGIEEAARIDGANKFVTF |
| | | | | | | | | | YKVVLPIVAPGIVATAIYTFINAWNEFLYALILINNT |
| | | | | | | | | | GKMTVAVALRSLNGSEILDWGDMMAASVIVVLPS |
| | | | | | | | | | IIFFSIIQNKIASGLSEGSVK |
| 135 | peg.7089 | SP_1715 | 0.083 | 6 | transport | | | NA | MFWNLVHYEFKNVNKWYLALYAAVLVLSALIGIQ |
| | | | | | | | | | TQGFKNLPYQESQATMLLFLATVFGGLMLTLAISTI |
| | | | | | | | | | FLIIKRFKGSVYDRQGYLTLTLPVSEHHIITAKLIGA |
| | | | | | | | | | FIWSLISTAVLALSAVIILALTAPEWIPLSYVITFVET |
| | | | | | | | | | HLPQIFLTGISFLLNTISGILCIYLAISIGQLFNEYRT |
| | | | | | | | | | ALAVAAYIGIQIVIGFIELFFNLSSNFYVNSLVGLNDH |
| | | | | | | | | | FYMGAGIAIVKELIFIAIFYLGTYYILRNKVNLL |
| 136 | peg.0790 | SP_1717 | 0.083 | 6 | transport | | | ABC transporter, | MRNMWVVIKETYLRHVKSWSFFFMVISPFLFLGIS |
| | | | | | | | | permease | VGIGHIQGSSMAKNNKVAVVTTVPSVAEGLKNVN |
| | | | | | | | | | GVNFDYKDEASAKEAIKEEKLKGYLTIDQEDSVLK |
| | | | | | | | | | AVYHGETSLENGIKFEVTGTLNELQNQLNRSTASL |
| | | | | | | | | | SQEQEKRLAQTIQFTEKIDEAKENKKFIQTIAAGAL |
| | | | | | | | | | GFFLYMILITYAGVTAQEVASEKGTKIMEVVFSSIR |
| | | | | | | | | | ASHYFYARMMALFLVILTHIGIYVVGGLAAVLLFK |
| | | | | | | | | | DLPFLAQSGILDHLGDAISLNTLLFILISLFMYVVLA |
| | | | | | | | | | AFLGSMVSRPEDSGKALSPLMILIMGGFFGVTALG |
| | | | | | | | | | AAGDNLLLKIGSYIPFISTFFMPFRTINGYAGGAEA |
| | | | | | | | | | WISLAITVIFAVVATGFIGRMYASLVLQTDDLGIWK |
| | | | | | | | | | TFKRALSYK |
| 137 | peg.0796 | n/a | 0.083 | 6 | CHO | | | pts system | MDYSKVAAEVIEAVGKDNLVAAAHCATRLRLVL |
| | | | | | transport | | | | KDEAKVNQAALDNNADVKGTFSTNGQYQIIIGPGD |
| | | | | | | | | | VNFVYAEIIKKTGLKEVSTDDLKEIANKDKKFNPL |
| | | | | | | | | | MDLIKLLSDIFVPIIPALVAGGLLMALRNFLTSPDLF |
| | | | | | | | | | GPQSIEDMYPAIKGFSAMIQLMSAAPFMFLPVLVGI |
| | | | | | | | | | SAAKRFGANQFLGAAIGMIMTTPDLGGKEAFWDIL |
| | | | | | | | | | GFHVTQTNYAYQVIPVLVAVWLLANLEKFFHKKL |
| | | | | | | | | | PSAVDFTFTPLLSVMITGFLTFTVIGPVMLVVSDAIT |
| | | | | | | | | | NAIVWLYNTTGAFGMGLFGGTYSLIVMTGLHQSFP |
| | | | | | | | | | AIETQLLSAYNNNGTGFGDYIFVVASMANVAQGA |
| | | | | | | | | | ATLAVYFLTKNAKTKGLSSSAAVSAFLGITEPALFG |
| | | | | | | | | | VNLKYKFPFFCALAGSAIGAFVAGLTHVIAVSLGA |
| | | | | | | | | | AGFIGFLSIKAGSIPMYIIAEIMSFVAAFAFTYFYGK |
| | | | | | | | | | TKAASVFADEAATATAETVTEPTVEAPVVEETDTL |
| | | | | | | | | | QNETLVTPIVGDVVALADVNDPVFSSGAMGQGIA |
| | | | | | | | | | VKPSQGVVYAPADAEVSIAFPTGHAFGLKTRNGAE |
| | | | | | | | | | VLIHVGIDTVSMNGDGFEAKVAQGNKVKAGDVLG |
| | | | | | | | | | TFDSNKIAAAGLDDTTMVIVTNTADYASVAPVAT |
| | | | | | | | | | GSVAKGDAVIEVKI |
| 138 | peg.0867 | n/a | 0.083 | 6 | transport | | | Sulfite exporter | VVEIIYFLIIIIASGLGSISGMGGGIIIKPLMDSFGYH |
| | | | | | | | | TauE/SafE | SVSDIAFYSSFSVFIMAIISTTKRFSQSKEIKWRLIFT |
| | | | | | | | | | VSFSSVLGGFLGHLIFQVLLSQLSVRLVSIVQMILLFV |
| | | | | | | | | | MLLVSFVLTDFKKTYQFDKIGFYMICGLLLGLISSF |
| | | | | | | | | | LGIGGGPLNVSLLMVFFSISIKEATMYSLAIIFFSQLS |
| | | | | | | | | | HLATIVVVTGLNQYHLAPVPVIFLASICGGVLGTV |
| | | | | | | | | | VSKVLPENWVRYCFKGMLFFVMGMTLYNLFHIL |
| 139 | peg.0870 | n/a | 0.083 | 6 | CHO | | | PTS system | MNTMLDKMQEKLSPIAMKVGNQKFLVALRDSFV |
| | | | | | transport | | | | GTMPVIMTGSIALLLNAFLVDLPQQFHLESITKTFQ |
| | | | | | | | | | WLVDINNLVFKGSIPIVSLLFIYCLGVNIAKIYKVDT |
| | | | | | | | | | VSAGLVSLASFVISIGSTVTKSFPLANVGDVKLDQI |
| | | | | | | | | | LQGIDNLAFDGKNLMVTIGNVIPGNHINARGYFTA |
| | | | | | | | | | MMIGFLASIIFCKVMKKNWVIKLPDSVPPAlAKPFT |
| | | | | | | | | | SIIPGFMAMYIVAILTYVFHLLSNDLLIDWVYKVLQ |
| | | | | | | | | | TPLLGLSQSFFAVILMIFLNKLFWFFGLHGGNVLAP |
| | | | | | | | | | IMEGLFGVAMLANLDAFQKGEPIPYIWTSGSFGAF |
| | | | | | | | | | VWFGGLGLVLAILIFSRNSHYRKVAKLGLAPVLFNI |
| | | | | | | | | | GEPVNYGLPVVLNPLLFIPFVLSPVFMATVAYWAT |
| | | | | | | | | | SWGLVSPVTQNVTWVMPPILYGFFSTAFDWRAIIL |
| | | | | | | | | | SVVCLIISVLTYFPFVKMAYKTELS |
| 140 | peg.0889 | SP_1823 | 0.083 | 6 | Metal | | | MgtC SapB family | MTATSLGLSNIEIVVRIVLSVVIGSIIGLERGSKSQPA |
| | | | | | transport | | | protein | GIRTYSIVCLAACLIMMTNEYVSYKFGTGDPTRLG |
| | | | | | | | | | AQVISGVGFLGAGTILITDKKKITGLTTAAGIWASA |
| | | | | | | | | | GIGLAIGVGFYEGALLVAISVWGVISMFQPLKKYL |
| | | | | | | | | | QNRSKMIELYIVVKSTEAYNRVLVYCAENGIRMTD |
| | | | | | | | | | SRTAFGDVNSDRIEYFDVPDKKIASFITLELSGRFEH |
| | | | | | | | | | LRLMEEIANIVGVIYVEEIS |
| 141 | peg.0893 | SP_1827 | 0.083 | 6 | regulation | | | Domain of unknown | MKNLTKIKFKENGEFNHFSGNTVVANLYTKQDLM |
| | | | | | | | | function (DUF1868) | EVVDIIQSRYRELPFIDKFTLTPRNSIHMTVIELLCH |
| | | | | | | | | | ENRETEFWSSNLPLDTPLQEIHDYFAKQLEIFPLLD |
| | | | | | | | | | EEIHMRITEMGKQNILVEPADEASAKRLEEIRTYVS |
| | | | | | | | | | EKAGVCFPNHDRYQFHISIGYLRIPLTEEEEEEFTKV |
| | | | | | | | | | RAELTEILLEKIPTITVNRIDYTVFEDMRQFVPYHEK |
| | | | | | | | | | FK |
| 142 | peg.0916 | SP_1850 | 0.083 | 6 | restriction | | | Type II restriction | MELHFNLELVETYKSNSQKARILTEDWVYRQSYCP |
| | | | | | | | | | NCGNNPLNHFENNRPVADFYCNHCSEEFELKSKK |
| | | | | | | | | | GNFSSTINDGAYATMMKRVQADNNPNFFFLTYTK |
| | | | | | | | | | NFEVNNFLVLPKQFVTPKSIIQRKPLAPTARRAGWI |
| | | | | | | | | | GCNIDLSQVPSKGRIFLVQDGQVRDPEKVTKEFKQ |
| | | | | | | | | | GLFLRKSSLSSRGWTIEILNCIDKIEGSEFTLEDMYR |
| | | | | | | | | | FESDLKNIFVKNNHIKEKIRQQLQILRDKEIIEFKGR |
| | | | | | | | | | GKYRKL |
| 143 | peg.1041 | SP_1952 | 0.083 | 6 | Protein | | | NA | MLKILNNSVIYLCLIPLLFLLLIIFPNDSLIYYFRLIL |
| | | | | | processing | | | | ISLISITLHELGHFLVGRCLSYKLEMLATPFFFYFRKK |
| | | | | | | | | | IYFKFPVLLAFGYCQMSNRNITNEKNSDRNLVFYFF |
| | | | | | | | | | GGGGANLIVAILALLGFVPYASEFFILNIILFLVTVC |
| | | | | | | | | | LPIDGTDGNAIREIVLYSKDSKTYQRFFANSLYNNP |
| | | | | | | | | | YITIDDFAKLTSKEKSFFSKFEKCLINLYFEVKGEGS |
| | | | | | | | | | AFTIANHEVFDSNIQENIIHEYYKLLHKSSDWIIPQN |
| | | | | | | | | | LSLEEVVFTLSNYIYSKNNKYLEKIKYLKKLVDFR |
| | | | | | | | | | QEEIIDFILNKEEVL |
| 144 | peg.1145 | SP_2058 | 0.083 | 6 | translation | | Yes | Exchanges the | MSDSPIKYRLIKKEKHTGARLGEIITPHGTFPTPMF |
| | | | | | | | | guanine residue with | MPVGTQATVKTQSPEELKEMGSGIILSNTYHLWLR |
| | | | | | | | | 7-amino methyl-7- | PGDELIARAGGLHKFMNWDQPILTDSGGFQVYSLA |
| | | | | | | | | deazaguanine in | DSRNITEEGVTFKNHLNGSKMFLSPEKAISIQNNLG |
| | | | | | | | | tRNAs with GU(N) | SDIMMSFDECPQFYQPYDYVKKSIERTSRWAERGL |
| | | | | | | | | anticodons (tRNA- | KAHRRPHDQGLFGIVQGAGFEDLRRQSAHDLVSM |
| | | | | | | | | Asp, -Asn, -His and | DFSGYSIGGLAVGETHEEMNAVLDFTTQLLPENKP |
| | | | | | | | | -Tyr). After this | RYLMGVGAPDSLIDGVIRGVDMFDCVLPTRIARNG |
| | | | | | | | | exchange, a | TCMTSQGRLVVKNAQFAEDFTPLDPECDCYTCNN |
| | | | | | | | | cyclopentendiol | YTRAYLRHLLKADETFGIRLTSYHNLYFLLNLMKQ |
| | | | | | | | | moiety is attached | VRQAIMDDNLLEFREYFVEKYGYNKSGRNF |
| | | | | | | | | tot he 7-aminomethyl | |
| | | | | | | | | group of 7- | |
| | | | | | | | | deazaguanine, | |
| | | | | | | | | resulting in the | |
| | | | | | | | | hypermodified | |
| | | | | | | | | nucleoside queuosine | |
| | | | | | | | | (Q) (7-(((4,5-cis- | |
| | | | | | | | | dihydroxy-2- | |
| | | | | | | | | cyclopenten-1- | |
| | | | | | | | | yl)amino)methyl)-7- | |
| | | | | | | | | deazaguanosine) (By | |
| | | | | | | | | similarity) | |
| 145 | peg.1174 | SP_2085 | 0.083 | 6 | transport | | | phosphate abc | MSKYLLKLLVYCFSALTFGSLFLIIGFILIKGLPHLSL |
| | | | | | | | | transporter | SLFSWTYTSENISLMPAIISTVILVFGALLLALPIGIF |
| | | | | | | | | | AGFYLVEYTKKDSLCVKIMRLASDTLSGIPSIVFGL |
| | | | | | | | | | FGMLFFVVFLGFQYSLLSGILTSVIMVLPVIIRSTEE |
| | | | | | | | | | ALLSVSDSMRQASYGLGAGKLRTVFRIVLPVAMP |
| | | | | | | | | | GILAGVILAIGRIVGETAALMYTLGTSTNTPSSLMS |
| | | | | | | | | | SGRSLALHMYMLSSEGLHVNEAYATGVILIITVLMI |
| | | | | | | | | | NTLSSLLSRKLVKGAS |
| 146 | peg.1194 | SP_2108 | 0.083 | 6 | transport | | | extracellular solute- | MSSKFMKSTAVLGTVTLASLLLVACGSKTADKPA |
| | | | | | | | | binding protein | DSDSSEVKELTVYVDEGYKSYIEEVAKAYEKEAG |
| | | | | | | | | family 1 | VKVTLKTGDALGGLDKLSLDNQSGNVPDVMMAP |
| | | | | | | | | | YDRVGSLGSDGQLSEVKLSDGAKTDDTTKSLVTA |
| | | | | | | | | | ANGKVYGAPAVIESLVMYYNKDLVKDAPKTFADL |
| | | | | | | | | | ENLAKDSKYAFAGEDGKTTAFLADWTNFYYTYGL |
| | | | | | | | | | LAGNGAYVFGQNGKDAKDIGLANDGSIAGINYAK |
| | | | | | | | | | SWYEKWPKGMQDTEGAGNLIQTQFQEGKTAAIID |
| | | | | | | | | | GPWKAQAFKDAKVNYGVATIPTLPNGKEYAAFGG |
| | | | | | | | | | GKAWVIPQAVKNLEASQKFVDFLVATEQQKVLYD |
| | | | | | | | | | KTNEIPANTEARSYAEGKNDELTTAVIKQFKNTQP |
| | | | | | | | | | LPNISQMSAVWDPAKNMLFDAVSGQKDAKTAAN |
| | | | | | | | | | DAVTLIKETIKQKFGE |
| 147 | peg.1276 | SP_2186 | 0.083 | 6 | CHO | | | Key enzyme in the | MSQEKYIMAIDQGTTSSRAIIFNKKGEKVSSSQKEF |
| | | | | | metabolism | | | regulation of | TQIFPQAGWVEHNANEIWNSVQSVIAGAFIESGVK |
| | | | | | | | | glycerol uptake and | PNQIEAIGITNQRETTVVWDKKTGLPIYNAIVWQSR |
| | | | | | | | | metabolism (By | QTAPLAEQLKSQGYVEKFHEKTGLIIDAYFSATKV |
| | | | | | | | | similarity) | RWILDHVEGAQERAEKGELLFGTIDTWLVWKLTD |
| | | | | | | | | | GAAHVTDYSNAARTMLYNIKELKWDDEILEILNIP |
| | | | | | | | | | KAILPEVRSNSEIYGKTAPFHFYGGEVPISGMAGDQ |
| | | | | | | | | | QAALFGQLAFEPGMVKNTYGTGSFIIMNTGEEMQL |
| | | | | | | | | | SENNLLTTIGYGINGKVYYALEGSIFIAGSAIQWLR |
| | | | | | | | | | DGLRMVENSPESEKYARDSHNNDEVYVVPAFTGL |
| | | | | | | | | | GAPYWNQNARGSVFGLTRGTSKEDFIKATLQSIAY |
| | | | | | | | | | QVRDIIDTMQVDTQTAIQVLKVDGGAAMNNFLMQ |
| | | | | | | | | | FQADILGIDIARAKNLETTALGAAFLAGLSVGYWK |
| | | | | | | | | | DLDELKLLNETGELFEPSMNESRKEQLYKGWKKA |
| | | | | | | | | | VKATQVFAEVDD |
| 148 | peg.1280 | n/a | 0.083 | 6 | adhesin | PspC | | NA | MRYSIRKFSVGVASVAVASLFMGSVVHATEKEGS |
| | | | | | | | | | TQAATSFNRGNGSQIERRKAAEQAIVSLSGYMTSL |
| | | | | | | | | | LNDGLDTSDSRFEELLKRAKGIVEEYRKKIDGASD |
| | | | | | | | | | REKIEKLQQEGQTKLDELVDKFKKGLSSSEQNGHA |
| | | | | | | | | | TPKDPSRNDGVQGDDSSVGQGEQPGASNQKNPES |
| | | | | | | | | | VPPTASGETDSPTKPKTSNDSVNSLERELKEVKETA |
| | | | | | | | | | KTTLNTYMLTRLKQENPGVFWFADLLRESKESVE |
| | | | | | | | | | KYKRMFDEASSKENVENLVKEAKQEIEALVVKHK |
| | | | | | | | | | GREIDLERTKAKTVIAKYLTGLLDDIKKNLKKEQHI |
| | | | | | | | | | NTVELIKKLGDIKRTYLYKLDESTQKAQLQELVTE |
| | | | | | | | | | SQSKLDEAFSKFKNGLSSSSSSGSSTKPETPQPEKPE |
| | | | | | | | | | HQKPTTPAPDTKPSPQPEGKKPSVPDINQEKEKAKL |
| | | | | | | | | | AVATYMSKILDDIQKHHLQKEKHRQIVALIKELDE |
| | | | | | | | | | LKKQALSEIDNVNTKVEIENTVHKIFADMDAVVTK |
| | | | | | | | | | FKKGLTQDTPKEPGNKKPSAPKPGMQPSPQPEVKP |
| | | | | | | | | | QLEKPKPEVKPQPEKPKPEVKPQLEKPKPEVKPQPE |
| | | | | | | | | | KPKPEVKPQLEKPKPEVKPQLEKPKPEVKPQPEKP |
| | | | | | | | | | KPEVKPQLEKPKPEVKPQPEKPKPEVKPQLEKPKPE |
| | | | | | | | | | VKPQPEKPKPEVKPQLEKPKPEVKPQLEKPKPEVK |
| | | | | | | | | | PQLEKPKPEVKPQPEKPKPEVKPQLEKPKPEVKPQP |
| | | | | | | | | | EKPKPEVKPQPEKPKPEVKPQLEKPKPDNSKPQAD |
| | | | | | | | | | DKKPSTTNNLSKDKQPSNQASTNEKATNKPKKSLP |
| | | | | | | | | | STGSISNLALEIAGLLTLAGATILAKKRMK |
| 149 | peg.1327 | SP_2233 | 0.083 | 6 | phage | | | domain protein | MESNLKKTTFIIVMIGISLIPALYNIIFLSSMWDPYG |
| | | | | | | | | | QLSDLPVAVVNNDKEASYNGNTMAIGKDMVSNL |
| | | | | | | | | | KENKTLDFHFVDEEEGKKGLEDGDYYMVVTLPSD |
| | | | | | | | | | LSEKATTLSNIQSTAAYQSLTSEQQTEISDSVSQNST |
| | | | | | | | | | DSIQSAQSIVALVQDLQGSLENLQNQSSNLSTLKNQ |
| | | | | | | | | | ANQVLPITSTSLIGLSSGLTEIQGAVTSKLVPASQSI |
| | | | | | | | | | ASGVNAYTTGVDKVSQGASQLSEKNATLTGSLDQ |
| | | | | | | | | | LVSGSNTLTQKSSSLTAGVGQLVEKTPELVSGIEKL |
| | | | | | | | | | STGSN |
| 150 | peg.1329 | n/a | 0.083 | 6 | phage | | | transposase IS116 | MKCFVGLDISSTKLDVCIMLSDTSTPVTASLSNDLS |
| | | | | | | | | IS110 IS902 family | GATEIKNHILDLNHTYHFERIVIGMEATSLYSFHPA |
| | | | | | | | | protein | MFFHEDRQLKELHVEIMVEQPNKIKKYRDAFEENK |
| | | | | | | | | | NDTIDAFYIADYFRIERFSPSFLKEEKYMALQHLTR |
| | | | | | | | | | TRLQIIEQLTRTKQHFIENIYYKCNTLAYQFKKEELS |
| | | | | | | | | | TNLWSSTMMTLMTDEMTLDELANMPLQELDDWL |
| | | | | | | | | | QKLGKGHFKDSEKLAQTIQAAIRGSYRLSKLQQDS |
| | | | | | | | | | VNVILGLLAREIRNLEKMIKEIDKAIEDMVETIPEYQ |
| | | | | | | | | | CLTSIPGVGKVYAAGIIAEIGQIERFKDHPQVAKFA |
| | | | | | | | | | GLNWREKQSGNSNSQHTSLVNRGNRYLRYYLVEA |
| | | | | | | | | | ANSVRRYDDEYKDFYKKKYHEVPKHQHKRAILLT |
| | | | | | | | | | ARKFVRLVDVLLRNHQLYTPPRRIMEDN |
| 151 | peg.1427 | SP_0091 | 0.083 | 6 | CHO | | | Binding-protein- | MAKKKIKKEKIDNVGIHSFSKKADIFFSIISGLIALSC |
| | | | | | transport | | | dependent transport | ILPFVFVIIISVTDEKSLLQYGYSFFPSQFGLDGFEFL |
| | | | | | | | | systems, inner | AQFKDKILQALFISVFVTVVGTLTNVFITTTYAYAIS |
| | | | | | | | | membrane | RTTFKYRRFFTIFVLLSMLFNAGLVPGYIMVTRVLQ |
| | | | | | | | | component | LGDTVWALIVPMLLSPFNIILMRSFFKKTIPEAILES |
| | | | | | | | | | ARIDGASEARIFFQICLPLSLPGIATITLLTALGFWN |
| | | | | | | | | | DWFNALLYIKSDNLYPLQYLLMQIQQNMDYIAKA |
| | | | | | | | | | VGLTGQLGVALPKETGRMAMVVVATLPIAILYPFF |
| | | | | | | | | | QRYFVKGLTIGGVKE |
| 152 | peg.1454 | n/a | 0.083 | 6 | unknown | | | NA | MKDDQKYLLAGLYSLLVAIFYFPLIESKGIFVSILM |
| | | | | | | | | | AVLLLYLIYFIGTVIHIVIIKFIRKKSFKYLVLYPFTY |
| | | | | | | | | | DGSWRFQPINLLYFPEMVRDVIPINLVQEYCQGQP |
| | | | | | | | | | YGLLKKMLKRIRLSREIALLLATVIVYFFTHRILPLS |
| | | | | | | | | | VFTFIFSYILLFAQSYLGGNTVWIGNRRLIIDDEFEKI |
| | | | | | | | | | LLSKSYIKEISSARYSEYLTCEYKNFTPIILLAIFENL |
| | | | | | | | | | LDSYLIQNQSKVDLDIFYKVLPLLYKEKYTMGFNY |
| | | | | | | | | | FVSLNYLLYKVGFLGIIYDNEALRDLSKQYLNKNIS |
| | | | | | | | | | ELQDGSFEGGIQDAVASKQIVVINEFIACLNSRCVP |
| | | | | | | | | | SQYDRFFYKDRPYIFSRKSPIKG |
| 153 | peg.1497 | SP_0156 | 0.083 | 6 | regulation | | | regulatoR | MYKVLLVDDEYMVTEGLKRLIPFDKWDMEVVAT |
| | | | | | | | | | ASHADEALEYVQENPVDVIISDVNMPDKTGLDMIR |
| | | | | | | | | | EMKEILPDAAYILLSGYQEFDYVKRAMNLSVVDY |
| | | | | | | | | | LVKPVDKVELGNLLEKIAGQLGERGKKSQTLSQEL |
| | | | | | | | | | DEAGFVSYLGDKENWWIGLSKEKQGSFTIPYYVLG |
| | | | | | | | | | QDWQIFISDQPLDGLVVTPFEAPYQEHFERWKLNA |
| | | | | | | | | | EKTLFYGSVNLQQSESLFAYYEPIYRVIIQGNLNQI |
| | | | | | | | | | VEELNLLEKVVLENTPRVPITKQLFIQFVMDVFHLF |
| | | | | | | | | | EHLKADDLTDIVKTIHAIQSFDELVPYIKETLTSFFG |
| | | | | | | | | | QYRMNENVVSVLEVIGRDYQKELSLKDISKALFIN |
| | | | | | | | | | PVYLGQLIKRETDSTFAELLNKQRIKAAQQLLLSTS |
| | | | | | | | | | DSIEDICYAVGYSNLGYFYKVFRKLCGKSPKAYRK |
| | | | | | | | | | QVETIL |
| 154 | peg.1506 | SP_0165 | 0.083 | 6 | bacteriocin | | | Flavoprotein | MDKKNLLVLVTGSVGASNVDTYLYYIKKFYNVKV |
| | | | | | | | | | ILSENSKKFISKELISYFCDKVYDEIFVEEVVPHVFL |
| | | | | | | | | | PYESDLLLILPATANVIGKIANGIADDLVTATVLNF |
| | | | | | | | | | NKKIIFCPNMNSTMWDNHIVQRNVSILKELGHIFLF |
| | | | | | | | | | ESKKTYEVGLRKAIDSTCSMLQPQSLVKELIKLENI |
| | | | | | | | | | VLEEGH |
| 155 | peg.1521 | SP_0182 | 0.083 | 6 | bacteriocin | | | peptidase U61 LD- | MVSTIGIVSLSSGIIGEDFVKHEVDLGIQRLKDLRLN |
| | | | | | | | | carboxypeptidase A | PIFLPHSLKGLDFIKDHPEARAEDLIHAFSDDSIDMI |
| | | | | | | | | | LCAIGGDDTYRLLPYLFENDQLQKVIKQKIFLGFSD |
| | | | | | | | | | TTMNHLMLHKLGIKTFYGQSFLADICELDKEMLA |
| | | | | | | | | | YSLHYFKELIETGRISEIRPSDIWYEERTDFSPTALG |
| | | | | | | | | | TPRVSHTNTGFDLLQGRAQFEGKILGGCLESLYDIF |
| | | | | | | | | | DNSRYADSTELCQKYKLFPDLSDWEGKILLLETSE |
| | | | | | | | | | EKPKPEDFKKMLLTLKDTGIFAVINGLLVGKPMDE |
| | | | | | | | | | TFHDDYKEALLDIIDSNIPIIYNLNVGHATPRAIVPF |
| | | | | | | | | | GVHAHVDAQEQVIRFDYNK |
| 156 | peg.1598 | SP_0267 | 0.083 | 6 | regulation | | | Luciferase-like | MVELGISTFGEITELEGTGQTYSHAERIRQLVAEIEL |
| | | | | | | | | | ADKVGLDVYGIGEHHRADFAVSAPEIVLAAGAVN |
| | | | | | | | | | TKKIRLTSAVSILSSMDPIRLFQQYATIDALSNGRSE |
| | | | | | | | | | IMAGRGSFTESFPLFGYDLKDYDSLFDEKLDLLQL |
| | | | | | | | | | VNEKTKLDWQGRLTQTIAGKEVYPRPVQDKLPLW |
| | | | | | | | | | IATGGHVESTVKIAQAGLPIVYAIIGGNPRYFKKLIQ |
| | | | | | | | | | AYREIGSEAGHADKDLKVGAHSWGWIAEDGEQA |
| | | | | | | | | | VKDYFHPTKQVVDAISKDRPHWQELRYEQYLEQV |
| | | | | | | | | | GPNGAMFVGNPDQVAEKLIRMIEDLGLDRFMLHL |
| | | | | | | | | | PLGSMPHDQVLRAIELFGTQVAPKVRAYFAMKEA |
| 157 | peg.1615 | SP_0285 | 0.083 | 6 | CHO | | | alcohol | MKAVVVNPESTGVAIEEKVLRPLETGEALVEVEYC |
| | | | | | metabolism | | | dehydrogenase | GVCHTDLHVAHGDFGQVPGRVLGHEGIGIVKEIAP |
| | | | | | | | | | DVKSLKVGDRVSVAWFFEGCGTCEYCTTGRETLC |
| | | | | | | | | | RTVKNAGYSVDGGMAEQCIVTADYAVKVPDGLD |
| | | | | | | | | | PAQASSITCAGVTTYKAIKEAKVEPGQWVVLYGA |
| | | | | | | | | | GGLGNLAVQYAKKVFNAHVIAVDINNDKLALAKE |
| | | | | | | | | | VGADIVINGLEVEDVAGLIKEKTDGGAHSAVVTAV |
| | | | | | | | | | SKVAFNQAVDSVRAGGRVVAVGLPSETMELSIVK |
| | | | | | | | | | TVLDGIQVIGSLVGTRKDLEEAFQFGAEGLVVPVV |
| | | | | | | | | | QKRPVEDAVAIFDEMEKGQIQGRMVLDFTH |
| 158 | peg.1638 | n/a | 0.083 | 6 | CHO | | | esterase | MSYIQLDFHSHSLNRNVNLNIFLPDGELLPAPEQPY |
| | | | | | metabolism | | | | PTIYFLPGFSASGKELSTFLHFRTQCLLNGIAVVIPD |
| | | | | | | | | | GDNSFYLDKPERLALYSQYVSQELIDYTCSILPLSH |
| | | | | | | | | | KREETYIGGISMGGYGALVHGFRYPEKFGKIVALS |
| | | | | | | | | | PAIDVAAILNGNASQFGTTFADLFKTEEDYYSSTDH |
| | | | | | | | | | PVNALKELLDKGEDIPELFIACGFQDELVYASNRSF |
| | | | | | | | | | AEELEKLGVNLTYIEDDGGHDVFFWDKYLPAVFEF |
| | | | | | | | | | LRK |
| 159 | peg.1662 | SP_0313 | 0.083 | 6 | metabolism | | | Glutathione | MTSLYDFSVLNQNNQATPLDSYRGKVLLIVNTATG |
| | | | | | | | | peroxidase | CGLTPQYQGLQELYERYQDQGFEILDFPCNQFMGQ |
| | | | | | | | | | APGSAEEINAFCSLHFQTTFPRFAKIKVNGKEADPL |
| | | | | | | | | | YVWLKDQKSGPLGKRVEWNFAKFLIGRDGQVFER |
| | | | | | | | | | FSSKTDPKQIEEAIQTLL |
| 160 | peg.1686 | SP_0338 | 0.083 | 6 | Protein | | | ATP-dependent Op | MNNNFNNFNNMDDLFNQLMGGMRGYSSENRRYL |
| | | | | | processing | | | protease ATP- | INGREVTPEEFAHYRATGQLPGNAETDVQMPQQA |
| | | | | | | | | binding subunit | SGMKQDGVLAKLGRNLTAEAREGKLDPVIGRNKE |
| | | | | | | | | | IQETSEILSRRTKNNPVLVGDAGVGKTAVVEGLAQ |
| | | | | | | | | | AIVNGDVPAAIKNKEIISIDISGLEAGTQYRGSFEEN |
| | | | | | | | | | VQNLVNEVKEAGNIILFFDEIHQILGAGSTGGDSGS |
| | | | | | | | | | KGLADILKPALSRGELTVIGATTQDEYRNTILKNAA |
| | | | | | | | | | LARRFNEVKVNAPSAENTFKILQGIRDLYQQHHNV |
| | | | | | | | | | ILPDEVLKAAVDYSVQYIPQRSLPDKAIDLVDVTA |
| | | | | | | | | | AHLAAQHPVTDVHAVEREIETEKDKQEKAVEAED |
| | | | | | | | | | FEAALNYKTRIAELEKKIENHTEDMKVTASVNDVA |
| | | | | | | | | | ESVERMTGIPVSQMGASDIERLKDMAHRLQEKVIG |
| | | | | | | | | | QDKAVEVVARAIRRNRAGFDEGNRPIGSFLFVGST |
| | | | | | | | | | GVGKTELAKQLALDMFGTQDAIIRLDMSEYSDRT |
| | | | | | | | | | AVSKLIGTTAGYVGYDDNSNTLTERVRRNPYSIILL |
| | | | | | | | | | DEIEKADPQVITLLLQVLDDGRLTDGQGNTVNFKN |
| | | | | | | | | | TVIIATSNAGFGYEANLTEDADKPELMDRLKPFFRP |
| | | | | | | | | | EFLNRFNAVIEFSHLTKEDLSKIVDLMLAEVNQTLA |
| | | | | | | | | | KKDIDLVVSQAAKDYITEEGYDEVMGVRPLRRVV |
| | | | | | | | | | EQEIRDKVTDFHLDHLDAKHLEADMEDGGLVIRE |
| | | | | | | | | | KS |
| 161 | peg.1741 | SP_0391 | 0.083 | 6 | adhesin | CbpF | | choline binding | MKLLKKMMQVALATFFFGLLGTSTVFADDSEGW |
| | | | | | | | | protein | QFVQENGRTYYKKGDLKETYWRVIDGKYYYFDPL |
| | | | | | | | | | SGEMVVGWQYIPAPHKGVTIGPSPRIEIALRPDWFY |
| | | | | | | | | | FGQDGVLQEFVGKQVLEAKTATNTNKHHGEEYDS |
| | | | | | | | | | PAEK |
| 162 | peg.1831 | SP_0488 | 0.083 | 6 | regulation | | Yes | PAP2 Family | MKGSFALLLFVILGYMVKFYPEMLVNFDQSIQTAI |
| | | | | | | | | | RGDLPDYLTILFRALTRLIDIPVIITWVVITAFVFYR |
| | | | | | | | | | KRWKIESFFMLGNLALAGLLIVTFKNIYQRPRPAIL |
| | | | | | | | | | HLVEEKGFSFPSGHSLAVTLMVGTLIVILSQRIKNP |
| | | | | | | | | | VWRKIVQIVLGLYLVSVLVSRVYLGVHYPSDVLAS |
| | | | | | | | | | LCVGLGVLFIEFPFYDKLRFQWRFKGKQK |
| 163 | peg.1915 | SP_0582 | 0.083 | 6 | nuclease | | | NA | MIKTFLSALSVILFVIPIITYSFFPSSNLNIWLSTRP |
| | | | | | | | | | ILAQIYAFPLATATMAAILSFVFFSLSFYKKNKQIRFY |
| | | | | | | | | | SGILLLLSLILLLFGTDKTLSSASNKTKTLKLVTWN |
| | | | | | | | | | VANQIEAQHIERIFSHFDADMAIFPELATNIRGEQE |
| | | | | | | | | | NQRIKLLFHQVGLSMANYDIFTSPPTDSGIAPVTVI |
| | | | | | | | | | VKKSYDFYTKAETFHTTRFGTIVLHSRKQDIPDIIAL |
| | | | | | | | | | HTAPPLPGLMEIWKQDLNIIHNQLASKYPKAIIAGD |
| | | | | | | | | | FNATMRHGALAKISSHRDALNALPPFERGTWNSQS |
| | | | | | | | | | PKLFNATIDHILLPKNHYYVKDLDIVSFQNSDHRCI |
| | | | | | | | | | FTEITF |
| 164 | peg.2014 | SP_0686 | 0.083 | 6 | bacteriocin | | | bacteriocin- | MKKLFILLSTFFLSFFLAWIIVLRAPQYLYASYDSVS |
| | | | | | | | | associated integral | LLRVKKDTQEPTREVFEQELENFANSEQSLIARRIV |
| | | | | | | | | membrane protein | EPSKDGTTHFTYATYGQGTLPKEFQEASQESRERS |
| | | | | | | | | | DPLNSYLLLSGSLTKEKLADKLGDLGYKASADRKI |
| | | | | | | | | | PPYFLAFRILLNPLILISLAIFGLSFFALVIITRIKEM |
| | | | | | | | | | RAAGIKLFSGQTLLSIMGHSLSTDIKWLLLSALLSFL |
| | | | | | | | | | GGGVVLFSQGLFYPILLATYGFGISFYLLFLLAISILL |
| | | | | | | | | | MLLYLMSLNYKALVPVIKGRLPLKRLMILTLLCQL |
| | | | | | | | | | VAVFTVGYAVKTGLTSYQRLKELEISKQAWQDRA |
| | | | | | | | | | DYYQISFGLGDRGKDTENQSKWYAFAKEAIEEEQ |
| | | | | | | | | | ALYVKDNLLHFANPQGKNEQGETLDTYSPDANTL |
| | | | | | | | | | YVSPSYLDKEKVVVDAETKQKLAHLQKGEFILLLP |
| | | | | | | | | | EHLRSREVELKKVFEERLSYYGKSGEEASAPLDYE |
| | | | | | | | | | MKAHVSYLSMGEKRFVYNNGENPVSTQYLTDPIL |
| | | | | | | | | | VVFTPTSTGDSFISLSSWSINAGKQLFIKGYESGLEL |
| | | | | | | | | | LKKVGIYEQVSYLKEGRSVYLTRYNEVQTETATLI |
| | | | | | | | | | LGAIVGIASSLLLFYSVNLLYFEQFRRDILIKRISGLR |
| | | | | | | | | | FFETHAQYMVSQFASFVFGASLFILSSRDLVIGLLT |
| | | | | | | | | | LLVFLASAVLTLYRQAQKESRVSMTIMKGK |
| 165 | peg.2025 | SP_0698 | 0.083 | 6 | transport | | | NA | MLKRFLALVWLRCQIILSNKSILLQVLVPFAFTYFY |
| | | | | | | | | | KYLMETQGKVNDQQALVLLMMCLPFSLALAVGSP |
| | | | | | | | | | ITIILSEEKEKYNLQTLLLSGVKGSEYILSTMFLPFLL |
| | | | | | | | | | TFVIMGTTPLILGVTIVHTFNYITIVLLTSLSIILFYL |
| | | | | | | | | | LIGLTAKSQVVAQVISLPAMILVAFLPMLSGLDKTV |
| | | | | | | | | | AKITDYSFMGLFTKFFTKWEGFSWNETLIPNLTLLI |
| | | | | | | | | | WIVLLLTLITITIRKKKIS |
| 166 | peg.2046 | SP_0720 | 0.083 | 6 | transport | | | ABC transporter, | MGLELRAIQSPIFSEPFDFTFHAQAFTLLVGSSGSGK |
| | | | | | | | | ATP-binding protein | SSLFQVIAQVSSLPYSGQVLIDGSEVSQLSIIARVQK |
| | | | | | | | | | VGILFQNPNHQFTMENLFEELIFTLENIGYHLQEIDS |
| | | | | | | | | | KIAEVVQQCRCEAILHRPIHHLSGGEKQKAALAVL |
| | | | | | | | | | FAMNPRVYLLDEPFASIDRKSRIEILEILKELALDGK |
| | | | | | | | | | TVILCDHDLSDYKAYIDHMVELRDGKLREVFQIPS |
| | | | | | | | | | YEMTQVASKEVASSPELFHMNRVTGELGNRPLFSI |
| | | | | | | | | | ADFTFYQGISCILGDNGVGKSTLFRSILQFQKYKGR |
| | | | | | | | | | IAWKGTVLKKKKSLYRDLTGVVQEAEKQFIRVSLR |
| | | | | | | | | | EELQLDGPDSERNQRIFQALRYFDLEQAVDKSPYQ |
| | | | | | | | | | LSGGQQKILQLLTILTSKASVILLDEPFAGLDDRAC |
| | | | | | | | | | HYFCKWIVEERNQGRSFLLISHRLDPLISVVDYWIE |
| | | | | | | | | | MTSQGLRHVKEVTITKPLTSQSSNTQGEVR |
| 167 | peg.2055 | SP_0729 | 0.083 | 6 | Metal | | Yes | p-type ATPase | MTEIVKASLENGIQKIRIRSEKGYHPAHIQLQKGIPA |
| | | | | | transport | | | | EITFHRATPSNCYKEILFEEEGILEPIGVDEEKVIRFT |
| | | | | | | | | | PQELGRHEFSCGMKMQKGSYTVVEKTRKSLSLLQ |
| | | | | | | | | | RFWITSIFTVPLVILMIGMLTGSISHQVMHWGTFLA |
| | | | | | | | | | TTPIMLVAGKPYIQSAWASFKKHNANMDTLVALG |
| | | | | | | | | | TLVAYFYSLVALFAGLPVYFESAGFILFFVLLGAVF |
| | | | | | | | | | EEKMRKNTSQAVEKLLDLQAKTAEVLSDDSYVQV |
| | | | | | | | | | PLEQVKVGDLIRVRPGEKIAVDGVVVEGVSSIDES |
| | | | | | | | | | MVTGESLPVDKTVGDTVIGSTINHSGTLVFRAEKV |
| | | | | | | | | | GSETVLAQIVDFVKKAQTSRAPIQDLTDKISGIFVP |
| | | | | | | | | | VVVILGIMTFWVWFVLLRDSVVVLGASFVSSLLYG |
| | | | | | | | | | VAVLIIACPCALGLATPTALMVGTGRSAKMGVLLK |
| | | | | | | | | | NGTVLQEIQKVQTLVFDKTGTLTEGKPVVTDVIGD |
| | | | | | | | | | EVEVFGLAASLEDASQHPLAEAIVKRASEAGLEFQ |
| | | | | | | | | | TVENFQTLHGKGVSGRINGKQVLLGNAKMLDGM |
| | | | | | | | | | DISNTYQDKLEELEKEAKTVVFLAVDNEIKGLLAL |
| | | | | | | | | | QDIPKENAKLAISQLKKRGLRTVMLTGDNAGVAR |
| | | | | | | | | | AIADQIGIEEVIAGVLPEEKAHEIHKLQQSGKVAFV |
| | | | | | | | | | GDGINDAPALSVADVGIAMGAGTDIAIESADLVLT |
| | | | | | | | | | TNNLLGVVRAFDMSKKTFHRILLNLFWAFIYNVVG |
| | | | | | | | | | IPIAAGVFSGVGLALNPELAGLAMAFSSVSVLTSSL |
| | | | | | | | | | LLNFSKID |
| 168 | peg.2083 | SP_0758 | 0.083 | 6 | CHO | | Yes | PTS System | MMKDTFKNVLSFEFWQKFGKALMVVIAVMPAAG |
| | | | | | transport | | | | LMISIGKSIVMINPTFTPLVITGGILEQIGWGVIGNLH |
| | | | | | | | | | ILFALAIGGSWAKERAGGAFAAGLAFILINRITGTIF |
| | | | | | | | | | GVSGDMLKNPDAMVTTFFGGSIKVADYFISVLEAP |
| | | | | | | | | | ALNMGVFVGIISGFVGATAYNKYYNFRKLPDALSF |
| | | | | | | | | | FNGKRFVPFVVILRSAIAAILLAAFWPVVQTGINNF |
| | | | | | | | | | GIWIANSQETAPILAPFLYGTLERLLLPFGLHHMLTI |
| | | | | | | | | | PMNYTALGGTYDILTGAAKGTQVFGQDPLWLAW |
| | | | | | | | | | VTDLVNLKGTDASQYQHLLDTVHPARFKVGQMIG |
| | | | | | | | | | SFGILMGVIVAIYRNVDADKKHKYKGMMIATALA |
| | | | | | | | | | TFLTGVTEPIEYMFMFIATPMYLVYSLVQGAAFAM |
| | | | | | | | | | ADVVNLRMHSFGSIEFLTRTPIAISAGIGMDIVNFV |
| | | | | | | | | | WVTVLFAVIMYFIANFMIQKFNYATPGRNGNYET |
| | | | | | | | | | AEGSEETSSEVKVAAGSQAVNIINLLGGRVNIVDV |
| | | | | | | | | | DACMTRLRVTVKDADKVGNAEQWKAEGAMGLV |
| | | | | | | | | | MKGQGVQAIYGPKADILKSDIQDILDSGEIIPETLPS |
| | | | | | | | | | QMTEAQQNTVHFKDLTEEVYSVADGQVVGLEQV |
| | | | | | | | | | KDPVFAQKMMGDGFAVEPANGNIVSPVSGTVSSIF |
| | | | | | | | | | PTKHAFGIVTEAGLEVLVHIGLDTVSLEGKPFTVHV |
| | | | | | | | | | AEGQKVAAGDLLVTADLDAIRAAGRETSTVVVFT |
| | | | | | | | | | NGDAIKSVKLEKTGSLAAKTAVAKVEL |
| 169 | peg.2115 | SP_0790 | 0.083 | 6 | transport | | | NA | MKKMKYYEETSALLHEFSEENQKYFEELWESFNL |
| | | | | | | | | | AGFLYDEDYLREQIYLMMLDFSEAERDGMSAEDY |
| | | | | | | | | | LGKNPKKIMKEILKGAPRSSIKESLLMPILVLAVLR |
| | | | | | | | | | YYQLLSDFSKGPLLTVNLLTFLGQLLIFLIGFGLVA |
| | | | | | | | | | TILRRSLVQDSPKMKIGTYIVVGTIVLLVVLGYVG |
| | | | | | | | | | MASFIQEGAFYIPAPWDSLSVFTISLVIGIWNWKEA |
| | | | | | | | | | VFRPFVSMIIAHLVVGSLLRYYEWMGISNVFLTKVI |
| | | | | | | | | | PSAVLFIGIFVLFRGFKKIKWSEV |
| 170 | peg.2138 | SP_0820 | 0.083 | 6 | Protein | | | ATP-dependent clp | MLCQNCKINDSTIHLYTNLNGKQKQIDLCQNCYKII |
| | | | | | processing | | | protease, ATP- | KTDPNNSLFKGMTDLNNRDFDPFGDFFNDLNNFRP |
| | | | | | | | | binding subunit | SSNTPPIPPTQSGGGYGGNGGYGSQNRGSAQTPPPS |
| | | | | | | | | | QEKGLLEEFGINVTEIARRGDIDPVIGRDDEIIRVIEI |
| | | | | | | | | | LNRRTKNNPVLIGEPGVGKTAVVEGLAQKIVDGD |
| | | | | | | | | | VPHKLQGKQVIRLDVVSLVQGTGIRGQFEERMQK |
| | | | | | | | | | LMEEIRKREDIILFIDEIHEIVGAGSASDGNMDAGNI |
| | | | | | | | | | LKPALARGELQLVGATTLNEYRIIEKDAALERRMQ |
| | | | | | | | | | PVKVDEPTVDETITILKGIQKKYEDYHHVQYTDAAI |
| | | | | | | | | | EAAATLSNRYIQDRFLPDKAIDLLDEAGSKMNLTL |
| | | | | | | | | | NFVDPKVIDQRLIEAENLKSQATREEDFEKAAYFR |
| | | | | | | | | | DQIAKYKEMQKKKITDQDTPIISEKTIEHIIEQKTNIP |
| | | | | | | | | | VGDLKEKEQSQLIHLAEDLKSHVIGQDDAVDKIAK |
| | | | | | | | | | AIRRNRVGLGTPNRPIGSFLFVGPTGVGKTELSKQL |
| | | | | | | | | | AIELFGSADSMIRFDMSEYMEKHSVAKLVGAPPGY |
| | | | | | | | | | VGYDEAGQLTEKVRHNPYSLILLDEVEKAHPDVM |
| | | | | | | | | | HMFLQVLDDGRLTDGQGRTVSFKDAIIIMTSNAGT |
| | | | | | | | | | GKTEASVGFGAAREGRTNSVLGELGNFFSPEFMNR |
| | | | | | | | | | FDGIIEFKALSKDNLLQIVELMLADVNKRLSSNNIR |
| | | | | | | | | | LDVTDKVKEKLVDLGYDPKMGARPLRRTIQDYIE |
| | | | | | | | | | DTITDYYLENPSEKDLKAVMTSKGNIQIKSAKKAE |
| | | | | | | | | | VKSSEKEK |
| 171 | peg.0038 | SP_0920 | 0.1 | 5 | metabolism | | | Catalyzes the | MKLEQVPTPAYVIDLAKLEANCRILQYVQEEAGCK |
| | | | | | | | | decarboxylation of | VLLAQKAYSLYKTYPLISQYLSGTTASGLYEAKLA |
| | | | | | | | | carboxynorspermidine | REEFPGEVHVFAPAFKDADLEELLEIMDHIVFNSER |
| | | | | | | | | and | QLRKHGPRCREAGVSVGLRLNPQCSTQGDHALYD |
| | | | | | | | | carboxy spermidine | PCAPGSRFGVTIDKIPSDLLDLVDGLHFHTLCEQGA |
| | | | | | | | | (By similarity) | DDLQTTLKAVEEQFGPYLHEVKWLNMGGGHHITR |
| | | | | | | | | | EGYDVDLLISEIKRIRKTYNLEIYIEPGEAIALNAGY |
| | | | | | | | | | LATEVLDIVENGMEILVLDASATCHMPDVLEMPYR |
| | | | | | | | | | PPLRNGFESQEKAHTYRLSSNTCLTGDVIGDYSFEN |
| | | | | | | | | | PVQIGDRLYFQDMAIYSFVKNNTFNGIGLPSLYLM |
| | | | | | | | | | DEQGDCSLLKAFGYQDFKGRLS |
| 172 | peg.0047 | SP_0930 | 0.1 | 5 | Cell wall | | | choline binding | MKKKLTSLALVGAFLGLSWYGNVQAQESSGNKIH |
| | | | | | processing | | | protein E | FINVQEGGSDAIILESNGHFAMVDTGEDYDFPDGS |
| | | | | | | | | | DSRYPWREGIETSYKHVLTDRVFRRLKELGVQKLD |
| | | | | | | | | | FILVTHTHSDHIGNVDELLSTYPVDRVYLKKYSDS |
| | | | | | | | | | RITNSERLWDNLYGYDKVLQTAAEKGVSVIQNITQ |
| | | | | | | | | | GDAHFQFGDMDIQLYNYENETDSSGELKKIWDDN |
| | | | | | | | | | SNSLISVVKVNGKKIYLGGDLDNVHGAEDKYGPLI |
| | | | | | | | | | GKVDLMKFNHHHDTNKSNTKDFIKNLSPSLIVQTS |
| | | | | | | | | | DSLPWKNGVDSEYVNWLKERGIERINAASKDYDA |
| | | | | | | | | | TVFDIRKDGFVNISTSYKPIPSFQAGWHKSAYGNW |
| | | | | | | | | | WYQAPDSTGEYAVGWNEIEGEWYYFNQTGILLQN |
| | | | | | | | | | QWKKWNNHWFYLTDSGASAKNWKKIAGIWYYF |
| | | | | | | | | | NKENQMEIGWIQDKEQWYYLDVDGSMKTGWLQY |
| | | | | | | | | | MGQWYYFAPSGEMKMGWVKDKETWYYMDSTG |
| | | | | | | | | | VMKTGEIEVAGQHYYLEDSGAMKQGWHKKAND |
| | | | | | | | | | WYFYKTDGSRAVGWIKDKDKWYFLKENGQLLVN |
| | | | | | | | | | GKTPEGYTVDSSGAWLVDVSIEKSATIKTTSHSEIK |
| | | | | | | | | | ESKEVVKKDLENKETSQHESVTNFSTSQDLTSSTSQ |
| | | | | | | | | | SSETSVNKSESEQ |
| 173 | peg.0250 | SP_1121 | 0.1 | 5 | CHO | | Yes | Catalyzes the | MDNREALKTFMTGENFYLQHYLGAHREELNGEY |
| | | | | | metabolism | | | formation of the | GYTFRVWAPNAQAVHLVGDFTNWIENQIPMVRND |
| | | | | | | | | alpha-1,6-glucosidic | FGVWEVFTNMAQEGHIYKYHVTRQNGHQLMKID |
| | | | | | | | | linkages in glycogen | PFAVRYEARPGTGAIVTELPDKKWRDGLWLARRK |
| | | | | | | | | by scission of a 1,4- | RWGFAERPVNIYEVHAGSWKRNPDGSPYSFAQLK |
| | | | | | | | | alpha-linked | DELIPYLVEMNYTHIEFMPLMSHPLGLSWGYQLM |
| | | | | | | | | oligosaccharide from | GYFALEHAYGRPEEFQDFVEECHTHNIGVIVDWVP |
| | | | | | | | | growing alpha-1,4- | GHFTINDDALAYYDGTPTFEYQDHNKAHNHGWG |
| | | | | | | | | glucan chains and | ALNFDLGKNEVQSFLISCIKHWIDVYHLDGIRVDA |
| | | | | | | | | the subsequent | VSNMLYLDYDDAPWTPNKDGGNLNYEGYYFLQR |
| | | | | | | | | attachment of the | LNEVIKLEYPDVMMIAEESSSATNITGMKEIGGLGF |
| | | | | | | | | oligosaccharide to | DYKWNMGWMNDILRFYEEDPIYRKYDFNLVTFSF |
| | | | | | | | | the alpha-1,6 | MYVFKENYLLPFSHDEVVHGKKSMMHKMWGDR |
| | | | | | | | | position | YNQFAGLRNLYTYQICHPGKKLLFMGSEYGQFLE |
| | | | | | | | | (By similarity) | WKSEEQLEWSNLEDPMNAKMKYFTSQLNQFYKD |
| | | | | | | | | | HRCLWEIDTSYDGIEIIDADNRDQSVLSFIRKGKKG |
| | | | | | | | | | EMLVCIFNMVPVERKDFTIGLPVAGIYEEVWNTEL |
| | | | | | | | | | EEWGGVWKEHNQTVQTQEGLWKDYEQTLTFTLP |
| | | | | | | | | | AMGASVWKIKRRLKSTKTVTNKNQKGVENEK |
| 174 | peg.0251 | SP_1122 | 0.1 | 5 | CHO | | | Catalyzes the | MKNEMLALILAGGQGTRLAKLTQSIAKPAVQFGG |
| | | | | | metabolism | | | synthesis of ADP- | RYRIIDFALSNCANSGIHNVGVVTQYQPLALNNHIG |
| | | | | | | | | glucose, a sugar | NGSSWGLDGIDSGVSILQPYSASEGNRWFEGTSHAI |
| | | | | | | | | donor used in | YQNIDYIDSVNPEYVLILSGDHIYKMDYDDMLQSH |
| | | | | | | | | elongation reactions | KDNNASLTVAVLDVPLKEASRFGIMNTDANNRIVE |
| | | | | | | | | on alpha-glucans (By | FEEKPAQPKSTKASMGIYIFDWQRLRNMLVAAEKS |
| | | | | | | | | similarity) | KVGMSDFGKNVIPNYLELGESVYAYEFSGYWKDV |
| | | | | | | | | | GTIESLWEANMEYISPENALDSRNRQWKIYSRNLIS |
| | | | | | | | | | PPNFLGANAHVEDSLVVDGCFVDGTVKHSILSTGA |
| | | | | | | | | | QVREGAEVLDSVIMSGAIIGQGAKIKRAIIGEGAIIS |
| | | | | | | | | | DGVEIDGTDEVQVVGYNEVVGVATDED |
| 175 | peg.0422 | n/a | 0.1 | 5 | CHO | | | Conserved Protein | MELVEFKNGHVNTVHEHIAITKVKEFLNNHDIPKD |
| | | | | | metabolism | | | | KGGIHKISTDFFYNIIDMETTTEENRPWESHKEYYD |
| | | | | | | | | | VHYLLNGEEIILYNFLSQMELSEYKVDDDWQQMN |
| | | | | | | | | | GTALFSIKLKKDMLLLLEPNDAHKTGLLVEEPINIK |
| | | | | | | | | | KVVFKVKI |
| 176 | peg.0438 | SP_1343 | 0.1 | 5 | transport | | | ABC transporter | MKDVVYSITPENKLSIDFSFKSPFRVLLTGTSGSGK |
| | | | | | | | | | TTILNLINGSLKPQKGYVNLLSHGKKSSDSIPTVDQ |
| | | | | | | | | | TPYIFDTTIRENVTLFQNEYFSDDQIIEVLKKVNLYE |
| | | | | | | | | | ELEKIDILNYQCGENGSNLSGGQKQKIALARALIRN |
| | | | | | | | | | NKVYLFDEISANLDNDNSNSIHDILFNLGISFIEVSH |
| | | | | | | | | | HYDLNDKRYTDI |
| 177 | peg.0440 | SP_1346 | 0.1 | 5 | bacteriocin | | | NA | MKRIIPVYIFQQVNVLLVSLYLLKFLCIGELTILQIL |
| | | | | | | | | | YGSSLISFLWMYGQRKQAHKVNMKSRMKWLGVE |
| | | | | | | | | | FVSLLIISLCFSLIHAQGSTNQANLIGLQHQIPWFSFL |
| | | | | | | | | | LFLINASMVEEFLYREILWNLVRNLDIRVALTSVLF |
| | | | | | | | | | ALAHHTGTILAWCLYVSLGMFLGMVRYKSDLWG |
| | | | | | | | | | SMGLHLVWNLLVYSLLLF |
| 178 | peg.0530 | SP_1450 | 0.1 | 5 | adhesin | | Yes | lipolytic protein | VAVQLLENWLLKEQEKIQTKYRHLNHISVVEPNIL |
| | | | | | | | | G-D-S-L family | FIGDSIVEYYPLQELFGTSKTIVNRGIRGYQTGLLLE |
| | | | | | | | | | NLDAHLYGGAVDKIFLLIGTNDIGKDVPVNEALNN |
| | | | | | | | | | LEAIIQSVARDYPLTEIKLLSILPVNEGEKYQQAVYI |
| | | | | | | | | | RSNEKIQNWNQAYQELASAYMQVEFVPVFNCLTD |
| | | | | | | | | | QAGQLKKEYTTDGLHLSIAGYQALSKSLKDYLY |
| 179 | peg.0635 | SP_1548 | 0.1 | 5 | unknown | | Yes | NA | MKRIVFELIFIATTWYIFLPPLNLTSWEFLFFLCGHL |
| | | | | | | | | | LVVAILFGFGKGINLVKTVHVRHGKAEAALNLEGF |
| | | | | | | | | | KINRLGKILLASIGGILLLAALVSLVTSSMFQAKNY |
| | | | | | | | | | ANVVTVTEKDFTEFPRSDTSKVPILDRSTAEKIGDR |
| | | | | | | | | | YLGSLTDKVSQYVAADTYTQLTIDGKPYRVTPLEY |
| | | | | | | | | | ADPIKWFNNQAKGIGEYIKVDMVTGNADLVDLKT |
| | | | | | | | | | PIKYSDSEYFNRDVKRHLRLKYPTKIFKTPSFEVDD |
| | | | | | | | | | EGNPFYVATVYQKQFGLAVPRPASVIILDATNGET |
| | | | | | | | | | KEYSLSDVPEWVDRIYPAEETIEQINYNGKYKDGF |
| | | | | | | | | | LNAMISKKNVTQTTKGYNYLSIGNDIYLYTGVTSA |
| | | | | | | | | | NADESNLGFILENMRTGEITKYSLASATEESARESA |
| | | | | | | | | | EGAVQEKSYKATFPILINLNDKPLYIMGLKDNAGL |
| | | | | | | | | | VKEYALVDAVEYQNVIVATTVEEMLSKYANKNDL |
| | | | | | | | | | EIDNATTESINGVVADLKSAVIKGDTVYFFKVDGKI |
| | | | | | | | | | YKVKASVSDDLPYLENGKTFEGQVGKDNYLKTFK |
| | | | | | | | | | LR |
| 180 | peg.0658 | SP_1573 | 0.1 | 5 | Cell wall | LytC | Yes | Putative cell wall | LKTKIGLASICLLGLATSHVAANETEVAKTSQDTTT |
| | | | | | processing | | | binding repeat | ASSSSEQNQSSNKTQTSAEVQTNAAAHWDGDYYV |
| | | | | | | | | | KDDGSKAQSEWIFDNYYKAWFYINSDGRYSQNEW |
| | | | | | | | | | HGNYYLKSGGYMAQNEWIYDSNYKSWFYLKSDG |
| | | | | | | | | | AYAHQEWQLIGNKWYYFKKWGYMAKSQWQGSY |
| | | | | | | | | | FLNGQGAMMQNEWLYDPAYSAYFYLKSDGTYAN |
| | | | | | | | | | QEWQKVGGKWYYFKKWGYMARNEWQGNYYLT |
| | | | | | | | | | GSGAMATDEVIMDGTRYIFAASGELKEKKDLNVG |
| | | | | | | | | | WVHRDGKRYFFNNREEQVGTEHAKKVIDISEHNG |
| | | | | | | | | | RINDWKKVIDENEVDGVIVRLGYSGKEDKELAHNI |
| | | | | | | | | | KELNRLGIPYGVYLYTYAENETDAESDAKQTIELIK |
| | | | | | | | | | KYNMNLSYPIYYDVENWEYVNKSKRAPSDTGTW |
| | | | | | | | | | VKIINKYMDTMKQAGYQNVYVYSYRSLLQTRLKH |
| | | | | | | | | | PDILKHVNWVAAYTNALEWENPHYSGKKGWQYT |
| | | | | | | | | | SSEYMKGIQGRVDVSVWY |
| 181 | peg.0697 | n/a | 0.1 | 5 | transport | | | Major Facilitator | MHIFLKNRAFRQLTVNEWISSFGDTIFYLAFINYVS |
| | | | | | | | | | SYAFAPLAIFLISLSETIPQVLQLFTGVIADFQKNRIS |
| | | | | | | | | | KYISILFIKVLLYSGVTLLLTSTDFSLFSVFFICSMNL |
| | | | | | | | | | ISDTIGFLAGYMLTPIYIRLINDDMTEAMGFRQSTSS |
| | | | | | | | | | IVRLIGNLSGGVFLGLFSISTLAFVNVLTFLFAFLGS |
| | | | | | | | | | LLIRNRLKKEEEKIEVPPYVGMSSFFQHLKESMKLL |
| | | | | | | | | | MTMEDVMVLLWILSISQAVLMMVEPVSAILLIHHP |
| | | | | | | | | | FMGLSTGQSLAILIMISLLHVILGGLLSGFLSKKISIR |
| | | | | | | | | | LNIYWSLLMESLIVIDFLRGSFLLILLGSAGDAFSAG |
| | | | | | | | | | VLSPRLQAMIFGIIPEELMGSVQSSINVINLLIPAVLS |
| | | | | | | | | | LALVFLATSAGLEVVAFALIILLLIAAYLVHQMKNL |
| | | | | | | | | | PNQEEV |
| 182 | peg.0820 | SP_1743 | 0.1 | 5 | regulation | | Yes | Methyltransferase | MATYETFAAVYDAVMDDSLYDKWTNFSLRHLPK |
| | | | | | | | | Type | TKERKKLLELACGTGIQSVRFSQAGFDVTGLDLSA |
| | | | | | | | | | DMLKIAEKRATSAKQKIAFIEGNMLNLSKAGKYDF |
| | | | | | | | | | VTCYSDSICYMQDEVEVGDVFKDVYNALNEEGVF |
| | | | | | | | | | IFDVHSTYQTDEVFPGYSYHENAEDFAMLWDTYE |
| | | | | | | | | | DEAPHSIVHELTFFIKEADGSFSRHDEVHEERTYEIL |
| | | | | | | | | | TYDILLEQAGFKSFKLYADFEDKEPTETSTRWFFV |
| | | | | | | | | | AQK |
| 183 | peg.0888 | SP_1821 | 0.1 | 5 | regulation | | Yes | Transcriptional | MKNINGKKVTIYDIARLSGFSPKTVSRVINGGVNV |
| | | | | | | | | regulator | KEETYQAIQKVIEELSYIPNAYAKNLTKKEAINILIS |
| | | | | | | | | | VKKIDSFPLIWFHTLLDKVLRTCKEFGVNAIVEYFG |
| | | | | | | | | | EEDTISNSIISSTGSLVDGVIVFYESVDDIRIQYLKKN |
| | | | | | | | | | HMPFLVFGESQTSGVVYVSNNNFQATYDMMKAV |
| | | | | | | | | | TEEKFKNMWLLMGGESHVNKDRERGVRSFLNDK |
| | | | | | | | | | NYFMDLKVIYGLSTIDSVYSYAMQHLTTQNYPDIIF |
| | | | | | | | | | VSGDEKVQGLIRACYEKGILIPDDISIIGFDNIPISQY |
| | | | | | | | | | YTPALSTIAPNYVKLAKEMIEGVLAIIKGESVTSVE |
| | | | | | | | | | VSPKFVRRQSF |
| 184 | peg.0927 | SP_1861 | 0.1 | 5 | Amino acid | | | ABC transporter | MIEYKNVALRYTEKDVLRDVNLQIEDGEFMVLVG |
| | | | | | transport | | | | PSGSGKTTMLKMINRLLEPTDGNIYMDGKRIKDYD |
| | | | | | | | | | ERELRLSTGYVLQAIALFPNLTVAENIALIPEMKGW |
| | | | | | | | | | SKEEITKKTEELLAKVGLPVAEYGHRLPSELSGGEQ |
| | | | | | | | | | QRVGIVRAMIGQPKIFLMDEPFSALDAISRKQLQVL |
| | | | | | | | | | TKELHKEFGMTTIFVTHDTDEALKLADRIAVLQDG |
| | | | | | | | | | EIRQVANPETILKAPATDFVADLFGGSVHD |
| 185 | peg.0934 | SP_1869 | 0.1 | 5 | Metal | | | permease protein | MKLSHYLIGLLLLLVFLSISIGTSDFSWGKLFDFDQ |
| | | | | | transport | | | | QTWLLFQESRLPRTISILLTASSMSMAGLLMQTITQ |
| | | | | | | | | | NQFAAPSTVGTTEAAKLGMVLSLFVFPSASLTQKM |
| | | | | | | | | | LFAFVSSIVFTLFFLAFMTIFTVKERWMLPLIGIIYSG |
| | | | | | | | | | IIGSVTEVIAYRFNLVQSMTAWTQGSFSMIQTHQYE |
| | | | | | | | | | WLFLGLIILITVWKLSQTFTIMNLGKETSESLGISYS |
| | | | | | | | | | LLEKLALFLVALTTSVTMITVGGLPFLGVIVPNLVR |
| | | | | | | | | | KRYGDNLSQTKLMVALVGANLVLACDILSRVLIRP |
| | | | | | | | | | YELSVSLLLGIIGSLVFILLLWRGGRKDAD |
| 186 | peg.1014 | SP_1925 | 0.1 | 5 | unknown | | | NA | MYICIDNSMTKNKELVKNQTFKPALLTRRLVKNF |
| | | | | | | | | | MIEEPDLARNPFTRPTLIDLDKVFMLDNTVIPTSYL |
| | | | | | | | | | ARRRRNVSEELYEEILDYLVQPRLISLNKSEFMQLN |
| | | | | | | | | | PGTY |
| 187 | peg.1055 | SP_1967 | 0.1 | 5 | signaling | | Yes | domain protein | MKKKIRWPLYVIAALIVTFLAFVVPLPYYIEVPGGS |
| | | | | | | | | | EDIRQVLKVNDTEDKEAGAYQFVTVGVQHATLAH |
| | | | | | | | | | MIYAWLTPFTDIRSAQETTGGSSDVEFMRINQFYM |
| | | | | | | | | | QTSQNMAKYQGLKTAGKDIELKYFGVYVLNVTD |
| | | | | | | | | | NSTFKGILNISDTVTAVNDQTFDSSKDLIDYVSSQK |
| | | | | | | | | | LGDSVKVTYEEDGQTKSAEGKIITLENGKNGIGIGL |
| | | | | | | | | | IDRTEVISNVPISFSTAGIGGPSAGLMFSLAIYTQIAH |
| | | | | | | | | | PDLRNGRIVAGTGTIDRDGNVGDIGGIDKKVVASA |
| | | | | | | | | | RAGAAIFFAPDNPVSEEEQKAHPDAKNNYQTALEA |
| | | | | | | | | | AKTIKTDMKIVPVKTLQDAIDYLKNNP |
| 188 | peg.1060 | SP_1972 | 0.1 | 5 | unknown | | Yes | Membrane | MNHTIIHDRAGLNQFYAKVYAFVGLGIGLSALVSG |
| | | | | | | | | | LMLTVFQSQLVYFLMQGRLWLTIATFAELALVFV |
| | | | | | | | | | ASSMASRNSPSALPVFLLYSVLNGFTLSFVVAFYTP |
| | | | | | | | | | GTVLSAFVSSALLFFVMAAVGIFTKKDLSGIGRAM |
| | | | | | | | | | MAALIGLLIAMVVNIFLASGFFDYMISVAMVLVFS |
| | | | | | | | | | GLIAWDNQKIRLAYEQSQGRVATGWVVSMALSIY |
| | | | | | | | | | LDFINLFLSILRIFGRND |
| 189 | peg.1195 | SP_2109 | 0.1 | 5 | transport | | | binding-protein- | MEKQQPSKAALLSIIPGLGQIYNKQKAKGFIFLGVT |
| | | | | | | | | dependent transport | IVFVLYFLALATPELSNLITLGDKPGRDNSLFMLIR |
| | | | | | | | | systems inner | GAFHLIFVIVYVLFYFSNIKDAHTIAKRINNGIPVPR |
| | | | | | | | | membrane | TLKDMIKGIYENGFPYLLIIPSYVAMTFAIIFPVIVTL |
| | | | | | | | | Component | MIAFTNYDFQHLPPNKLLDWVGLTNFTNIWSLSTF |
| | | | | | | | | | RSAFGSVLSWTIIWALAASTLQIVIGIFTAIIANQPFI |
| | | | | | | | | | KGKRIFGVIFLLPWAVPAFITILTFSNMFNDSVGAIN |
| | | | | | | | | | TQVLPILAKFLPFLDGALIPWKTDPTWTKIALIMMQ |
| | | | | | | | | | GWLGFPYIYVLTLGILQSIPNDLYEAAYIDGANAW |
| | | | | | | | | | QKFRNITFPMILAVAAPTLISQYTFNFNNFSIMYLFN |
| | | | | | | | | | GGGPGSVGGGAGSTDILISWIYRLTTGTSPQYSMA |
| | | | | | | | | | AAVTLIISIIVISISMIAFKKLHAFDMEDV |
| 190 | peg.1450 | n/a | 0.1 | 5 | CHO | | | Inherit from NOG: | MRYDFGKVYKEIRESKGLTQEEVCGGVLSRTSLSK |
| | | | | | metabolism | | | Transcriptional | IESGKTTPKYENMEFLLRQINMSFEEFEYICQLYQP |
| | | | | | | | | regulator | SQRTEIMQTYLNMRSIIGTSDLVNLFQKCQDYLKT |
| | | | | | | | | | HHDLPIEEIRDMLEVVIYIRQHGAGELSDHAEQVV |
| | | | | | | | | | KKLWRKIEKQDTWYESDLKILNTILFSFPIEYLHLIT |
| | | | | | | | | | GKILQRLEVYKNYQHLYDLRIAILLNLSTLYLYNQ |
| | | | | | | | | | DKNMCKQICYTLLEDAKNKKSYDRLAICYVRIGIC |
| | | | | | | | | | TDDSKLIQKGFSLLELTEETSMLSHLKKEVEIYYQA |
| | | | | | | | | | KER |
| 191 | peg.1459 | n/a | 0.1 | 5 | unknown | | | NA | MELVLPNNYVVIDEEEMMYLDGGAYLSKSACQGI |
| | | | | | | | | | CVALAMSPGTFIALTGAAVLTKKLINYIKVGGLGG |
| | | | | | | | | | WLIGAAAGVLATAAGKIAYCIGYGALNRGCDISGN |
| | | | | | | | | | PYPWDGFISATVR |
| 192 | peg.1493 | SP_0152 | 0.1 | 5 | transport | | Yes | ABC transporter, | MESLIQTYLPNVYKMGWAGQAGWGTAIYLTLYM |
| | | | | | | | | permease | TVLSFIIGGFLGLVAGLFLVLTAPGGVLENKVVFWI |
| | | | | | | | | | LDKITSIFRAVPFIILLAILSPLSHLIVKTSIGPNAAL |
| | | | | | | | | | VPLSFAVFAFFARQVQVVLAELDGGVIEAAQASGAT |
| | | | | | | | | | FWDIVGVYLSEGLPDLIRVTTVTLISLVGETAMAG |
| | | | | | | | | | AVGAGGIGNVAIAYGFNRYNHDVTILATIIIILIIFAI |
| | | | | | | | | | QFLGDFLTKKLSHK |
| 193 | peg.1494 | SP_0153 | 0.1 | 5 | unknown | | | integral membrane | MKKSILTTLLFAVLYFLCMGIGVLLGNLFDQTGNM |
| | | | | | | | | protein TIGR02185 | FYAPAFTALVGGSVYMILVAKVPRFGAITTIGLVIA |
| | | | | | | | | | FFFLGTKHGAGSFLPGIICGLLADEVAHLGKYKDK |
| | | | | | | | | | TKNFLSFIIFAFSTTGPILLMWIAPKAYMATLLARG |
| | | | | | | | | | KSQEYIDRIMVAPNPGTVLLFIASIVIGALVGALIGQ |
| | | | | | | | | | ALSKKFAQKI |
| 194 | peg.1509 | SP_0167 | 0.1 | 5 | transport | | Yes | Major Facilitator | MKNLIKLLIIRLIVNLADSVFYIVALWHVSNNYSSS |
| | | | | | | | | | MFLGIFIAVNYLPDLLLIFFGPVIDRVNPQKILIISIL |
| | | | | | | | | | VQLAVAVIFLLLLNQISFWVIMSLVFISVMASSISYVI |
| | | | | | | | | | EDVLIPQVVEYDKIVFANSLFSISYKVLDSIFNSFAS |
| | | | | | | | | | FLQVAVGFILLVKIDIGIFLLALFILLLLKFRTSNANI |
| | | | | | | | | | ENFSFKYYKREVLQGTKFILNNKLLFKTSISLTLINF |
| | | | | | | | | | FYSFQTVVVPIFSIRYFDGPIFYGIFLTIAGLGGILGN |
| | | | | | | | | | MLAPIVIKYLKSNQIVGVFLFLNGSSWLVAIVIKDY |
| | | | | | | | | | TLSLILFFVCFMSKGVFNIIFNSLYQQIPPHQLLGRV |
| | | | | | | | | | NTTIDSIISFGMPIGSLVAGTLIDLNIELVLIAISIPY |
| | | | | | | | | | FLFSYIFYTDNGLKEFSIY |
| 195 | peg.1535 | SP_0200 | 0.1 | 5 | unknown | | Yes | NA | MSVYGRVEEVHKENREPLEYQIEQESHHRESSRLP |
| | | | | | | | | | LVKILLWSTLVTGITLGVPLLLDLMSAQEVQDFYA |
| | | | | | | | | | GWALHQTGKIYSDYYGSQGLLYYLLTYVSQGGFF |
| | | | | | | | | | FAIFEWLALVAGGFFLFRSADTLTEQGDQAGHLVT |
| | | | | | | | | | IFYMLVTGLAFGGGYATLLALPFLFAAFSLVAAYL |
| | | | | | | | | | SNPSHDKGFVRIGLALAGGFFFAPLSSLLFIAVVSL |
| | | | | | | | | | GLLVFNLGHRRFAHGFYQFLAVALGFSLVFYPTAY |
| | | | | | | | | | YSAATGSFGDAISGIRYPIDSIRFDFTSKILENMFFY |
| | | | | | | | | | GLLSLGLGFVVCIFLGLFQSKPFKLYVISVPASLVVI |
| | | | | | | | | | LGLILLFFSQEPLHASYLMVVFPVFLLLLVTNIKSQ |
| | | | | | | | | | QRGRSARRSLRETPVSLWSRFFKGNLYLLVFGFVY |
| | | | | | | | | | LLSVPFLMKFVLYPVPYQERNRLADLVKEETNTED |
| | | | | | | | | | AIYAWDDTATLYRKSERLSPSAILSPLHYTATEENR |
| | | | | | | | | | NKLLNDLKEKQPKVIVVNDKVVVWSEVETLLKEN |
| | | | | | | | | | YQQVKTDYSEFKVYKIK |
| 196 | peg.1636 | n/a | 0.1 | 5 | transport | | | pts system | MSKYNVLAKDIVTYVGGAENVKSLRHCATRLRFE |
| | | | | | | | | | LKDESQADKEALMNLDILQVVQASGQYQVVVGPH |
| | | | | | | | | | VASVYEAVMAELPVIGGASSINEDTTAEEKKWYD |
| | | | | | | | | | VIFDTISGSFTPLIPVFCGSGLVKALGSVLLMTGLLT |
| | | | | | | | | | AESSTYAVLNAAGNAVFYFLPILLAISVANKLGTNP |
| | | | | | | | | | YIAGAIGAALLEPSFTALAGNESNTFLGLPIIINNYA |
| | | | | | | | | | SSVFPAFMAVVGLYFLDKFLKKVVPATIHLVVLPFI |
| | | | | | | | | | EIVAMVPLTIFIFGPFGTYLSTGLSMFVTNLLDLNA |
| | | | | | | | | | MLAGAVLSMIWIYVVMLGLHWALIPIMINNIATNG |
| | | | | | | | | | SDPLMGLMIGTVWVAGGVSLGVALKAKDKKLKGI |
| | | | | | | | | | AWTGMIPCFLSGVSEPIMYGILFRYKKSLMWATV |
| | | | | | | | | | MNAVTGAMAGLIGISGTQIAGGVFTIPTFSPILGYL |
| | | | | | | | | | ALIAVSVFGTAALIVIFGFGEEGSKA |
| 197 | peg.1660 | SP_0311 | 0.1 | 5 | unknown | | | NA | MSLDIDKEKMTIMGIAFENRSVFKSVWYALSTNMI |
| | | | | | | | | | EGWRPTVSDVEKLRDEALSLGMT |
| 198 | peg.1670 | SP_0322 | 0.1 | 5 | bacteriocin | | Yes | Glycosyl Hydrolase | MIKKVTIEKIKSPERFLEVPLLTKEEVGQAIDKVIRQ |
| | | | | | | | | Family 88 | LELNLDYFKEDFPTPATFDNVYPIMDNTEWTNGF |
| | | | | | | | | | WTGELWLAYEYSQQDAFKNIAHKNVLSFLDRVNK |
| | | | | | | | | | RVELDHHDLGFLYTPSCMAEYKINGDGEAREATL |
| | | | | | | | | | KAADKLIERYQEKGGFIQAWGDLGKKEHYRLIIDC |
| | | | | | | | | | LLNIQLLFFAYQETGDQKYYDIAESHFYASANNVIR |
| | | | | | | | | | DDASSFHTFYFDPETGQPFKGVTRQGYSDDSCWA |
| | | | | | | | | | RGQSWGVYGIPLTYRHLKDESCFDLFKGVTNYFLN |
| | | | | | | | | | RLPKDHVSYWDLIFNDGSAQSRDSSATAIAVCGIH |
| | | | | | | | | | EMLKHLPEVDADKDIYKHAMHAMLRSLIEHYAND |
| | | | | | | | | | QFTPGGTSLLHGVYSWHSGKGVDEGNIWGDYYYL |
| | | | | | | | | | EALIRFYKDWNLYW |
| 199 | peg.1677 | SP_2241 | 0.1 | 5 | unknown | | | NA | MKSLARLLIIHVFISIFLFFALISGAVSHTVLLLLLLF |
| | | | | | | | | | LPALNKGLEKIQSKRIPVLNAALFFLLISFPQLLTNP |
| | | | | | | | | | VQWKFSIFLVVTIISSLAYFYNFYQVVKEVDQKQLI |
| 200 | peg.1875 | SP_0536 | 0.1 | 5 | unknown | | | NA | MRIKEFEDDDLVSPRTNQLMFIGLTGFMSIICLYRGI |
| | | (weak) | | | | | | | TAGESYQQLIAYIGAILCLIIMLLLIWGLKYYKK |
| 201 | peg.1877 | SP_0545 | 0.1 | 5 | bacteriocin | | Yes | caax amino terminal | MKKYQLLFKISAVFSYLFFVFGLSQLTLIVQNYWQ |
| | | | | | | | | protease family | FSSQIGNLFWIQNILSLLFIGVMIVVLVKTGHGYLFR |
| | | | | | | | | protein | IPRKKWLWYSILTVLVVVFQISFNVQTAKHVQSTA |
| | | | | | | | | | EGWAVLIGYSGTNFAELGIYIALFFLVPLMEELIYR |
| | | | | | | | | | GLLQHAFFKHSRFGLDLLLPSILFALPHFLSLPSLLD |
| | | | | | | | | | IFVFATVGIIFAGLTRYTKSIYPSYAVHVINNIVATFP |
| | | | | | | | | | FLLTFLHRVLG |
| 202 | peg.1928 | SP_0599 | 0.1 | 5 | transport | | | ABC transporter, | MNPIQRSWAYVSRKRLRSFILFLILLVLLAGISACLT |
| | | | | | | | | permease | LMKSNKTVESNLYKSLNTSFSIKKIENGQTFKLSDL |
| | | | | | | | | | ASVSKIKGLENVSPELETVAKLKDKEAVTGEQSVE |
| | | | | | | | | | RDDLSAADNNLVSLTALEDSSKDVTFTSSAFNLKE |
| | | | | | | | | | GRHLQKGDSKKILIHEELAKKNGLSLHDKIGLDAG |
| | | | | | | | | | QSESGKGQTVEFEIIGIFSGKKQEKFTGLSSDFSENQ |
| | | | | | | | | | VFTDYESSQTLLGNSEAQVSAARFYVENPKEMDG |
| | | | | | | | | | LMKQVENLALENQGYQVEKENKAFEQIKDSVATF |
| | | | | | | | | | QTFLTIFLYGMLIAGAGALILVLSLWLRERVYEVGI |
| | | | | | | | | | LLALGKGKSSIFLQFCLEVVLVSLGALLPAFVAGN |
| | | | | | | | | | GITTYLLQTLLASGDQASLQDTLAKASSLSTSILSFA |
| | | | | | | | | | ESYVFLVLLSCLSVALCFLFLFRKSPKEILSSIS |
| 203 | peg.2015 | SP_0687 | 0.1 | 5 | Peptide | | | abc transporter atp- | MIELKNISKKFGSRQLFSDTNLHFEGGKIYALIGTS |
| | | | | | transport | | | binding protein | GCGKTTLLNMIGRLEPYDKGQIIYDGTSLKDIKPSV |
| | | | | | | | | | FFRDYLGYLFQDFGLIESQTVKENLNLGLVGKKLK |
| | | | | | | | | | EKEKISLMKQALNRVNLSYLDLKQPIFELSGGEAQ |
| | | | | | | | | | RVALAKIILKDPPLILADEPTASLDPKNSEELLSILES |
| | | | | | | | | | LKNPNRTIIIATHNPLIWEQVDQVIRVTDLSHR |
| 204 | peg.2041 | SP_0719 | 0.1 | 5 | Metal | | | TENA THI-4 family | METQDYAFQPGLTVGELLKSSQKDWQAAINHRFV |
| | | | | | transport | | | protein | KELFAGTIENKVLKDYLIQDYHFFDAFLSMLGACV |
| | | | | | | | | | AHADKLESKLRFAKQLGFLEADEDGYFQKAFKEL |
| | | | | | | | | | KVAENDYLEVTLHPVTKAFQDLMYSAVASSDYAH |
| | | | | | | | | | LLVMLVIAEGLYLDWGSKDLALPEVYIHSEWINLH |
| | | | | | | | | | RGPFFAEWVQFLVDELNRVGKNREDLTELQQRWN |
| | | | | | | | | | QAVALELAFFDIGYNV |
| 205 | peg.2157 | SP_0840 | 0.1 | 5 | unknown | | | NA | MKILKRYILELCFILSFALPFIKGANADNGRCFVET |
| | | (weak) | | | | | | | YYGFTFLMEHAIVTAVFIYSFLIAFLLKKRWAKWI |
| | | | | | | | | | AAGSYCFLVLWIATEGYFFRMSLEDLIRLWTSLEIL |
| | | | | | | | | | TQTYQLGFYLNILLGILLIIKYFKVKQ |

*in van Opijnen and Camilli (2012) Genome Res. 22(12):2541-2551
**Protein functions were predicted with the eggNOG pipeline, using HMMR on the bacteria database.
https://academic.oup.com/mbe/article/34/8/2115/3782716

### 3. Vaccine Compositions

Vaccine compositions are provided that comprise at least one immunogenic polypeptide comprising at least one S. pneumoniae protein that is required for or involved in mammalian transmission of S. pneumoniae. In some embodiments, the S. pneumoniae protein required for or involved in mammalian transmission of S. pneumoniae has an amino acid sequence set forth as any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, and 205, or an immunogenic fragment or variant of any thereof, and a non-naturally occurring pharmaceutically acceptable carrier.

In certain embodiments, the vaccine composition comprises an immunogenic polypeptide comprising a S. pneumoniae protein (or an immunogenic fragment or variant thereof) that is required for or involved in mammalian transmission of S. pneumoniae and is naturally expressed on the surface of at least one strain of S. pneumoniae. Alternatively, the S. pneumoniae protein is naturally expressed on the surface of at least one strain of S. pneumoniae when the bacterium is undergoing autolysis. In particular embodiments, the vaccine composition comprises an immunogenic polypeptide comprising a S. pneumoniae protein (or an immunogenic fragment or variant thereof) that is required for or involved in mammalian transmission of S. pneumoniae, wherein the immunogenic polypeptide does not comprise a transmembrane domain.

In certain embodiments, the vaccine composition comprises an immunogenic polypeptide comprising a S. pneumoniae choline-binding protein or an immunogenic fragment or variant thereof. In some of these embodiments, the choline-binding protein has an amino acid sequence selected from the group consisting of SEQ ID NO: 27, 39, and 82; or an immunogenic fragment or variant of any thereof.

In other embodiments, the vaccine composition comprises an immunogenic polypeptide comprising a S. pneumoniae sensor kinase of the competence cascade (ComD), the homolog of putative C3-degrading protease (CppA), or the iron transporter PiaA, or an antigenic fragment or variant of any thereof. In some of these embodiments, the ComD protein has the amino acid sequence set forth as SEQ ID NO: 92 or an immunogenic fragment or variant thereof. In other embodiments, the CppA protein has the amino acid sequence set forth as SEQ ID NO: 44 or an immunogenic fragment or variant thereof. In still other embodiments, the PiaA protein has the amino acid sequence set forth as SEQ ID NO: 10 or an immunogenic fragment or variant thereof.

A “vaccine composition” is a formulation containing at least one immunogenic polypeptide comprising at least one S. pneumoniae protein that is required for or involved in mammalian transmission of S. pneumoniae in a form suitable for administration to a subject that results in a reduction in the transmissibility of S. pneumoniae upon infection.

As used herein, the terms “peptide,” “polypeptide,” or “protein” are used interchangeably herein and are intended to encompass a singular “polypeptide” as well as plural “polypeptides,” and refers to a molecule composed of monomers (amino acids) linearly linked by amide bonds (also known as peptide bonds). The terms “peptide” and “polypeptide” refer to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “peptide” and “polypeptide”. The terms apply to amino acid polymers in which one or more amino acid residues is an artificial chemical analogue of a corresponding naturally occurring amino acid, as well as to naturally occurring amino acid polymers. Non-limiting examples of artificial amino acid residues include norleucine and selenomethionine. An amino acid residue is a molecule having a carboxyl group, an amino group, and a side chain and having the generic formula H₂NCHRCOOH, where R is an organic substituent, forming the side chain. An amino acid residue, whether it is artificial or naturally occurring, is capable of forming a peptide bond with a naturally occurring amino acid residue.

The immunogenic polypeptides used in the presently disclosed compositions and methods can be recombinantly produced, chemically synthesized, or purified from a biological sample. In some embodiments, the immunogenic polypeptide is an isolated polypeptide.

An “isolated” or “purified” peptide is substantially or essentially free from components that normally accompany or interact with the peptide as found in its naturally occurring environment. Thus, an isolated or purified peptide is substantially free of other cellular material, or culture medium when produced by recombinant techniques, or substantially free of chemical precursors or other chemicals when chemically synthesized. A peptide that is substantially free of cellular material includes preparations of peptide having less than about 30%, 20%, 10%, 5%, or 1% (by dry weight) of contaminating protein. When the peptide is recombinantly produced, optimally culture medium represents less than about 30%, 20%, 10%, 5%, or 1% (by dry weight) of chemical precursors or non-peptide-of-interest chemicals.

The presently disclosed invention involves immunogenic fragments and variants of the various S. pneumoniae proteins. Such immunogenic fragments can comprise at least about 5, at least about 10, at least about 15, at least about 20, at least about 50, at least about 60, at least about 80, at least about 100, at least about 150, at least about 200, at least about 300, at least about 400, at least about 500, at least about 600, at least about 700, at least about 800, at least about 900, at least about 1,000 contiguous amino acid residues or up to the entire contiguous amino acid residues of the protein. Methods for obtaining such fragments are known in the art and are described in further detail elsewhere herein.

By “variant” is intended substantially similar sequences. Thus, immunogenic variants include sequences that are functionally equivalent to the protein sequence of interest and retain immunogenic activity. Generally, amino acid sequence variants of the invention will have at least 40%, at least about 50%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to a respective amino acid sequence. Methods of determining sequence identity are also discussed elsewhere herein.

While the proteins identified in the presently disclosed screens were derived from the ferret-transmissible BHN97 (serotype 19F) strain, the immunogenic polypeptides used in the presently disclosed compositions and methods may be derived from any strain or serotype of S. pneumoniae. There are more than 90 serotypes known, with the most commonly used vaccines targeting 13 (1, 3, 4, 5, 6A, 6B, 7F, 9V, 14, 19A, 19F, 18C, and 23F) or 23 of these (1, 2, 3, 4, 5, 6B, 7F, 8, 9N, 9V, 10A, 11A, 12F, 14, 15B, 17F, 18C, 19F, 19A, 20, 22F, 23F, and 33F). Thus, in some embodiments, the immunogenic S. pneumoniae polypeptides are derived from any one of serotypes 1, 2, 3, 4, 5, 6A, 6B, 7F, 8, 9N, 9V, 10A, 11A, 12F, 14, 15B, 17F, 18C, 19A, 19F, 20, 22F, 23F, and 33F, or a combination thereof. In some embodiments, the immunogenic polypeptide is one that is conserved in sequence (fully conserved or comprise conservative amino acid differences) among two or more of the sequenced strains of S. pneumoniae. The BHN97 (serotype 19F) genome can be found at NCBI Accession No. PRJNA420094.

With respect to the amino acid sequences for the various full length polypeptides, variants include those polypeptides that are derived from the native polypeptides by deletion (so-called truncation) or addition of one or more amino acids to the N-terminal and/or C-terminal end of the native polypeptide; deletion or addition of one or more amino acids at one or more sites in the native polypeptide; or substitution of one or more amino acids at one or more sites in the native polypeptide. Such variants may result from, for example, genetic polymorphism or from human manipulation. Methods for mutagenesis and nucleotide sequence alterations are well known in the art. See, for example, Walker and Gaastra, eds. (1983) Techniques in Molecular Biology (MacMillan Publishing Company, New York); Kunkel (1985) Proc. Natl. Acad. Sci. USA 82:488-492; Kunkel et al. (1987) Methods Enzymol. 154:367-382; Sambrook et al. (1989) Molecular Cloning: A Laboratory Manual (2d ed., Cold Spring Harbor Laboratory Press, Plainview, N.Y.); U.S. Pat. No. 4,873,192; and the references cited therein; herein incorporated by reference. Guidance as to appropriate amino acid substitutions that may not affect biological activity of the various proteins may be found in the model of Dayhoff et al. (1978) Atlas of Polypeptide Sequence and Structure (Natl. Biomed. Res. Found., Washington, D.C.), herein incorporated by reference. Conservative substitutions, such as exchanging one amino acid with another having similar properties, may be preferred. The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays.

By “sequence identity” is intended the same nucleotides or amino acid residues are found within the variant sequence and a reference sequence when a specified, contiguous segment of the nucleotide sequence or amino acid sequence of the variant is aligned and compared to the nucleotide sequence or amino acid sequence of the reference sequence. Methods for sequence alignment and for determining identity between sequences are well known in the art. With respect to optimal alignment of two nucleotide sequences, the contiguous segment of the variant nucleotide sequence may have additional nucleotides or deleted nucleotides with respect to the reference nucleotide sequence. Likewise, for purposes of optimal alignment of two amino acid sequences, the contiguous segment of the variant amino acid sequence may have additional amino acid residues or deleted amino acid residues with respect to the reference amino acid sequence. The contiguous segment used for comparison to the reference nucleotide sequence or reference amino acid sequence will comprise at least 20 contiguous nucleotides, or amino acid residues, and may be 30, 40, 50, 100, or more nucleotides or amino acid residues. Corrections for increased sequence identity associated with inclusion of gaps in the variant's nucleotide sequence or amino acid sequence can be made by assigning gap penalties. Methods of sequence alignment are well known in the art.

The determination of percent identity between two sequences can be accomplished using a mathematical algorithm. For example, percent identity of an amino acid sequence can be determined using the Smith-Waterman homology search algorithm using an affine 6 gap search with a gap open penalty of 12 and a gap extension penalty of 2, BLOSUM matrix 62. Alternatively, percent identity of a nucleotide sequence is determined using the Smith-Waterman homology search algorithm using a gap open penalty of 25 and a gap extension penalty of 5. Such a determination of sequence identity can be performed using, for example, the DeCypher Hardware Accelerator from TimeLogic Version G. The Smith-Waterman homology search algorithm is taught in Smith and Waterman (1981) Adv. Appl. Math 2:482-489, herein incorporated by reference. Alternatively, the alignment program GCG Gap (Wisconsin Genetic Computing Group, Suite Version 10.1) using the default parameters may be used. The GCG Gap program applies the Needleman and Wunch algorithm and for the alignment of nucleotide sequences with an open gap penalty of 3 and an extend gap penalty of 1 may be used. Another preferred, non-limiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and) (BLAST programs of Altschul et al. (1990) J. Mol. Biol. 2/5:403. BLAST nucleotide searches can be performed with the NBLAST program, score=100, wordlength 12, to obtain nucleotide sequences having sufficient sequence identity. BLAST protein searches can be performed with the) (BLAST program, score=50, wordlength=3, to obtain amino acid sequences having sufficient sequence identity. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST) can be used. See www.ncbi.nlm.nih.gov. Another non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

The presently disclosed vaccine compositions comprise immunogenic polypeptides. The term “immunogenic” or “immunogenic activity” refers to the ability of a polypeptide to elicit an immunological response in a subject (e.g., a mammal). An immunological response to a polypeptide is the development in an animal of a cellular and/or antibody-mediated immune response to the polypeptide. Usually, an immunological response includes but is not limited to one or more of the following effects: the production of antibodies, B cells, helper T cells, suppressor T cells and/or cytotoxic T cells, directed to an epitope or epitopes of the polypeptide. The term “epitope” refers to the site on an antigen to which specific B cells and/or T cells respond so that antibody is produced. The immunogenicity of a polypeptide can be assayed for by measuring the level of antibodies or T cells produced against the polypeptide. Assays to measure for the level of antibodies are known, for example, see Harlow and Lane (1988) Antibodies, A Laboratory Manual (Cold Spring Harbor Publications, New York), for a standard description of antibody generation, immunoassay formats and conditions that can be used to determine specific immunoreactivity. Assays for T cells specific to a polypeptide are known. See, for example, Rudraraju et al. (2011) Virology 410:429-36, herein incorporated by reference.

In some embodiments, the immunogenic polypeptide comprises a fusion protein. In some of these embodiments, the fusion protein comprises not only the S. pneumoniae protein that is required for or involved in mammalian transmission, but also an additional S. pneumoniae immunogen (such as one that inhibits colonization), a peptide adjuvant, a tag or a combination thereof. Adjuvants generally are substances that can enhance the immunogenicity of polypeptides. Adjuvants may play a role in both acquired and innate immunity (e.g., toll-like receptors) and may function in a variety of ways, not all of which are understood. For example, the peptide adjuvant can comprise at least one of a tetanus toxoid, pneumolysis keyhole limpet hemocyanin or the like. Conjugation may be direct or indirect (e.g., via a linker). A tag may be N-terminal or C-terminal For instance, tags may be added to polypeptide to facilitate purification, detection, solubility, or confer other desirable characteristics on the protein. For instance, a purification tag may be a peptide, oligopeptide, or polypeptide that may be used in affinity purification. Examples include His, GST, TAP, FLAG, myc, HA, MBP, VSV-G, thioredoxin, V5, avidin, streptavidin, BCCP, Calmodulin, Nus, S tags, lipoprotein D, and β-galactosidase.

In certain embodiments, a S. pneumoniae protein or immunogenic fragment or variant thereof is covalently bound to another molecule. This may, for example, increase the half-life, solubility, bioavailability, or immunogenicity of the antigen. Molecules that may be covalently bound to the antigen include a carbohydrate, biotin, poly(ethylene glycol) (PEG), polysialic acid, N-propionylated polysialic acid, nucleic acids, polysaccharides, and PLGA. There are many different types of PEG, ranging from molecular weights of below 300 g/mol to over 10,000,000 g/mol. PEG chains can be linear, branched, or with comb or star geometries. In some embodiments, the naturally produced form of a protein is covalently bound to a moeity that stimulates the immune system. An example of such a moeity is a lipid moeity. In some instances, lipid moieties are recognized by a Toll-like receptor (TLR) such as TLR2, and activate the innate immune system.

The presently disclosed vaccine compositions comprise an immunogenic polypeptide and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. In certain embodiments, the presently disclosed vaccine compositions comprise a non-naturally occurring pharmaceutically acceptable carrier. That is, a carrier that is not normally found in nature or not normally found in nature in combination with the immunogenic polypeptide.

In one embodiment, the vaccine composition is in bulk or in unit dosage form. The unit dosage form is any of a variety of forms, including, for example, a capsule, an IV bag, a tablet, a single pump on an aerosol inhaler or a vial. The quantity of active ingredient in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this invention include powders, sprays, ointments, pastes, creams, lotions, gels, solutions, patches and inhalants. In one embodiment, the active compound (i.e., immunogenic polypeptide comprising S. pneumoniae protein or variant or fragment thereof) is mixed under sterile conditions with a pharmaceutically acceptable carrier, and with any preservatives, buffers or propellants that are required. In specific embodiments the immunogenic polypeptide is administered as a solution, dispersion, suspension, powder, capsule, tablet, pill, time release capsule, time release tablet, and/or time release pill.

Moreover, the administration may be by continuous infusion or by single or multiple boluses. In specific embodiments, an immunogenic polypeptide can be infused over a period of less than about 4 hours, 3 hours, 2 hours or 1 hour. In still other embodiments, the infusion occurs slowly at first and then is increased over time.

Vaccine compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In some embodiments, the vaccine composition is formulated for intranasal administration (i.e., inhalation) or pulmonary delivery. For administration by inhalation, the compounds are delivered in the form of an aerosol spray from pressured container or dispenser, which contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The presently disclosed vaccine compositions may comprise an adjuvant or an additional S. pneumoniae immunogen (such as one that inhibits colonization), which can be fused to the immunological polypeptide as described elsewhere herein. Alternatively, the vaccine compositions can be administered along with an adjuvant or an additional S. pneumoniae immunogen (such as one that inhibits colonization), through either simultaneous or subsequent administration. Many substances, both natural and synthetic, have been shown to function as adjuvants. For example, adjuvants may include, but are not limited to, mineral salts, squalene mixtures, muramyl peptide, saponin derivatives, mycobacterium cell wall preparations, certain emulsions, mineral gels such as aluminum hydroxide, surface active substances such as lysolecithin, pluronic polyols, polyanions, peptides, oil or hydrocarbon emulsions, dinitrophenol, monophosphoryl lipid A, mycolic acid derivatives, nonionic block copolymer surfactants, Quil A, complete Freund's adjuvant, incomplete Freund's adjuvant, cholera toxin B subunit, polyphosphazene and derivatives, immunostimulating complexes (ISCOMs), cytokine adjuvants, MF59 adjuvant, lipid adjuvants, mucosal adjuvants, certain bacterial exotoxins and other components, certain oligonucleotides, PLG, and others.

4. Methods for Reducing the Transmissibility of S. pneumoniae and Incidence Rate of Invasive Disease

Methods are provided for reducing the mammalian transmission of S. pneumoniae by administering to a mammalian subject infected with S. pneumoniae or at risk of infection by S. pneumoniae a vaccine composition comprising at least one immunogenic polypeptide comprising at least one S. pneumoniae protein that is required for or involved in mammalian transmission of S. pneumoniae. In some embodiments, the S. pneumoniae protein required for or involved in mammalian transmission of S. pneumoniae has an amino acid sequence set forth as any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, and 205, or an immunogenic fragment or variant of any thereof, and a non-naturally occurring pharmaceutically acceptable carrier.

As used herein, a “mammalian subject” can be any mammal, e.g., a human, primate, bird, mouse, rat, fowl, dog, cat, cow, horse, goat, rabbit, camel, sheep or a pig. In certain embodiments, the mammal is a human. In some embodiments, the human being administered the vaccine composition can be a newborn, infant, toddler, preadolescent, adolescent, or adult.

In particular embodiments, the mammalian subject is infected with S. pneumoniae or at risk of infection by S. pneumoniae. Although any individual has a certain risk of becoming infected with S. pneumoniae, certain sub-populations have an increased risk of infection. Those with a higher risk of infection include, but are not limited to, mammals whose immune system is compromised and/or have chronic illnesses, newborns, infants, toddlers, seniors, children or adults with asplenia, splenic dysfunction, sickle-cell disease, cochlear implants or cerebrospinal fluid leaks, childcare workers, and healthcare workers.

As used herein, the term “transmission” refers to the mammal-to-mammal spread of S. pneumoniae by direct contact with respiratory secretions, such as saliva or mucus. In some embodiments, the presently disclosed compositions and methods reduce transmission of S. pneumoniae from a mother to an offspring—prenatally, postnatally, or both.

The compositions and methods disclosed herein result in the reduced mammalian transmission of S. pneumoniae. Specifically, those mammals that have been administered the vaccine compositions disclosed herein are less likely to transmit S. pneumoniae if or when they are infected with the bacteria than a mammal that has not received the vaccine composition. In some embodiments, the transmission rate from a vaccinated mammal or population thereof to another vaccinated or non-vaccinated mammal or population thereof is reduced by at least about 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100% when compared to a mammal that has not received the vaccine composition disclosed herein or a population thereof. A “mammalian population” refers to a group of more than one mammal.

The mammalian transmission rate can be measured using any method known in the art, including those described in the examples. For example, mammalian transmission rates can be determined by measuring the colonization of S. pneumoniae within members of a population comprising at least one individual subject that has been infected with S. pneumoniae and wherein all other members of the population have been brought into physical contact with the infected individual(s), followed by determining the infection burden of the contact mammals by quantification of viable bacteria present in the anterior nares by nasal lavage of the nares and culturing lavage fluid, or by direct contact of the anterior nares with bacteriological growth media.

In another aspect, methods are provided for reducing the mammalian transmissibility of S. pneumoniae by reducing the levels or activity of a S. pneumoniae protein required for or involved in mammalian transmission of S. pneumoniae. In some embodiments, the S. pneumoniae protein required for or involved in mammalian transmission of S. pneumoniae has an amino acid sequence set forth as any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, and 205.

In yet another aspect, methods are provided for reducing the mammalian transmissibility of S. pneumoniae by increasing the levels or activity of a S. pneumoniae protein that decreases tolerance of desiccation stress.

As used herein, “desiccation” refers to the state of S. pneumoniae outside of a liquid culture at ambient temperatures and humidity levels. As used herein, “tolerance of desiccation stress” refers to the ability of S. pneumoniae to remain viable after desiccation. In some embodiments, S. pneumoniae may remain viable after at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 1.5 days, 2 days, 2.5 days, 3 days, 3.5 days, 4 days, 4.5 days, 5 days, 5.5 days, 6 days, 6.5 days, 1 week, 1.5 weeks, 2 weeks, 2.5 weeks, 3 weeks, 3.5 weeks, 1 month, 1.5 months, 2 months, 3 months or more.

In some embodiments, the tolerance of desiccation stress is reduced in S. pneumoniae with increased levels or activity of a S. pneumoniae protein that decreases tolerance of desiccation stress such that the length of desiccation after which the engineered S. pneumoniae is still viable is reduced by at least about 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100% when compared to a proper control S. pneumoniae (e.g., a S. pneumoniae of the same strain in which the levels or activity of these proteins have not been manipulated by the hand of man).

In some embodiments, the S. pneumoniae protein that decreases tolerance of desiccation stress is a spxB protein or a spxR protein. In some of these embodiments, the spxB protein has an amino acid sequence set forth as SEQ ID NO: 228 or a variant or fragment thereof.

SEQ ID NO: 228
MTQGKITASAAMLNVLKTWGVDTIYGIPSGTLSSLMDALAEDKDIRFLQV
RHEETGALAAVMQAKFGGSIGVAVGSGGPGATHLINGVYDAAMDNTPFLA
ILGSRPVNELNMDAFQELNQNPMYNGIAVYNKRVAYAEQLPKVIDEACRA
AVSKKGPAVVEIPVNFGFQEIDENSYYGSGSYERSFIAPALNEVEIDKAV
EILNNAERPVIYAGFGGVKAGEVITELSRKIKAPIITTGKNFEAFEWNYE
GLTGSAYRVGWKPANEVVFEADTVLFLGSNFPFAEVYEAFKNTEKFIQVD
IDPYKLGKRHALDASILGDAGQAAKAILDKVNPVESTPWWRANVKNNQNW
RDYMNKLEGKTEGELQLYQVYNAINKHADQDAIYSIDVGNTTQTSTRHLH
MTPKNMWRTSPLFATMGIALPGGIAAKKDNPDRQVWNIMGDGAFNMCYPD
VITNVQYDLPVINLVFSNAEYGFIKNKYEDTNKHLFGVDFTNADYAKIAE
AQGAVGFTVDRIEDIDAVVAEAVKLNKEGKTVVIDARITQHRPLPVEVLE
LDPKLHSEEAIKAFKEKYEAEELVPFRLFLEEEGLQSRAIK

In other embodiments, the spxR protein has an amino acid sequence set forth as SEQ ID NO: 229 or a variant or fragment thereof.

SEQ ID NO: 229
MSKHQEILSYLEELPVGKRVSVRSISNHLGVSDGTAYRAIKEAENRGIVE
TRPRSGTIRVKSQKVAIERLTFAEIAEVTSSEVLAGQEGLEREFSKFSIG
AMTEQNILSYLHDGGLLIVGDRTRIQLLALENENAVLVTGGFQVHDDVLK
LANQKGIPVLRSKHDTFTVATMINKALSNVQIKTDILTVEKLYRPSHEYG
FLRETDTVKDYLDLVRKNRSSRFPVINQHQVVVGVVTMRDAGDKSPSTTI
DKVMSRSLFLVGLSTNIANVSQRMIAEDFEMVPVVRSNQTLLGVVTRRDV
MEKMSRSQVSALPTFSEQIGQKLSYHHDEVVITVEPFMLEKNGVLANGVL
AEILTHMTQDLVVNSGRNLIIEQMLIYFLQAVQIDDILRIQARIIHHTRR
SAIIDYDIYHGHQIVSKANVTVKIN

As used herein, “mammalian transmissibility” refers to the ability of a S. pneumoniae bacterium to be transmitted from one infected mammal to another mammal by direct contact with respiratory secretions, such as saliva or mucus.

In some embodiments, the mammalian transmissibility of a S. pneumoniae in which the levels or activity of at least one S. pneumoniae protein required for or involved in mammalian transmission of S. pneumoniae is reduced, or a S. pneumoniae in which the levels or activity of at least one S. pneumoniae protein that decreases tolerance of desiccation stress is increased, is reduced by at least about 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100% when compared to a proper control S. pneumoniae (e.g., a S. pneumoniae of the same strain in which the levels or activity of these proteins have not been manipulated by the hand of man).

In some embodiments, the levels or activity of a S. pneumoniae protein is specifically increased or reduced. As used herein, the term “specifically” means the ability of a molecule or method to increase or reduce the levels or activity of a S. pneumoniae protein without impacting the level or activity of other proteins.

Methods of increasing levels of a S. pneumoniae protein are known in the art and include bacterial transformation of a polynucleotide encoding the S. pneumoniae protein or an active variant or fragment thereof or the introduction of the protein itself or an active variant or fragment thereof. Alternatively, the expression level of the gene encoding the S. pneumoniae protein can be activated by introducing transcription factors that activate the promoter regulating the transcription of the gene.

In some embodiments, levels of the S. pneumoniae protein can be reduced by reducing the expression of a gene encoding the same by any method known in the art. For example, the expression of a gene can be reduced by using antisense RNA, by knocking out the gene, using RNA-guided CRISPR enzymes, such as a nuclease deficient Cas enzyme that is fused to a transcriptional repressor domain, engineered zinc finger nucleases, transcription activator-like effector nucleases (TALEN5), or peptide nucleic acids.

Reduction (i.e., decreasing) of the levels of a S. pneumoniae protein can be achieved by any means known in the art. For example, gene expression can be decreased by a mutation. The mutation can be an insertion, a deletion, a substitution or a combination thereof, provided that the mutation leads to a decrease in the expression of the S. pneumoniae protein. In specific embodiments, recombinant DNA technology can be used to introduce a mutation into a specific site on the chromosome. Such a mutation may be an insertion, a deletion, a replacement of one nucleotide by another one or a combination thereof, as long as the mutated gene leads to a decrease in the expression of a S. pneumoniae protein. Such a mutation can be made by deletion of a number of base pairs. In one embodiment, the deletion of one single base pair could render a gene encoding a S. pneumoniae protein non-functional, thereby decreasing the levels of a S. pneumoniae protein, since as a result of such a mutation, the other base pairs are no longer in the correct reading frame. In other embodiments, multiple base pairs are removed, such as about 2, 5, 10, 20, 30, 40, 50, 75, 100, 150, 200, 300, 400, 500, or more base pairs. In still other embodiments, the entire length of the gene encoding a S. pneumoniae protein is deleted. Mutations introducing a stop-codon in the open reading frame, or mutations causing a frame-shift in the open reading frame could be used to reduce the expression of a gene encoding a S. pneumoniae protein.

Other techniques for decreasing the expression of a gene encoding a S. pneumoniae protein are well-known in the art. For example, techniques may include modification of the gene by site-directed mutagenesis, restriction enzyme digestion followed by re-ligation, PCR-based mutagenesis techniques, allelic exchange, allelic replacement, RNA-guided CRISPR enzymes, or post-translational modification. Standard recombinant DNA techniques are all known in the art and described in Maniatis/Sambrook (Sambrook, J. et al. Molecular cloning: a laboratory manual. ISBN 0-87969-309-6). Site-directed mutations can be made by means of in vitro site directed mutagenesis using methods well known in the art.

Inhibitory molecules, such as inhibitory small molecules, nucleic acid molecules, such as antisense RNA, ribozymes, peptides, antibodies, antagonists, aptamers, and peptidomimetics that reduce the levels or activity of a S. pneumoniae protein can be introduced into S. pneumoniae cells using any method known in the art for introduction of molecules into bacterial cells. By “introducing” is intended presenting to the bacterial cell the expression cassette, mRNA, or polypeptide in such a manner that the sequence gains access to the interior of the bacterial cell. The methods provided herein do not depend on a particular method for introducing an expression cassette or sequence into a bacterial cell, only that the polynucleotide or polypeptide gains access to the interior of at least one bacterial cell. Methods for introducing sequences into bacterial cells are known in the art and include, but are not limited to, stable transformation methods, transient transformation methods, and virus-mediated methods.

In specific embodiments, the levels or activity of a S. pneumoniae protein is reduced or increased by at least about 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100% when compared to a proper control S. pneumoniae (e.g., a S. pneumoniae of the same strain in which the levels or activity of these proteins have not been manipulated by the hand of man). Gene or protein expression can be measured by any means known in the art.

Methods are also provided for reducing the incidence rate of at least one invasive disease caused by S. pneumoniae in a mammalian population by administering to at least one mammalian subject within the mammalian population a vaccine composition comprising at least one immunogenic polypeptide comprising at least one S. pneumoniae protein that is required for or involved in mammalian transmission of S. pneumoniae. In some embodiments, the S. pneumoniae protein required for or involved in mammalian transmission of S. pneumoniae has an amino acid sequence set forth as any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, and 205, or an immunogenic fragment or variant of any thereof, and a non-naturally occurring pharmaceutically acceptable carrier.

The incidence rate of any invasive disease caused by S. pneumoniae within a population can be reduced by the presently disclosed methods and compositions. S. pneumoniae is considered “invasive” when it is found in the blood, cerebrospinal fluid, pleural fluid, joint fluid, peritoneal fluid, or other normally sterile sites. Non-limiting examples of invasive diseases caused by S. pneumoniae include pneumonia, otitis media, bacterial meningitis, bacteremia, sinusitis, septic arthritis, osteomyelitis, peritonitis, sepsis, and endocarditis.

As used herein, “incidence rate” refers to the numbers or percentage of subjects within a population that have newly acquired an invasive disease. Administering to at least one mammalian subject within a mammalian population a presently disclosed vaccine composition can reduce the incidence rate within the population of an invasive disease caused by S. pneumoniae by at least about 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100% when compared to a mammalian population in which none of the individual mammals were administered a presently disclosed vaccine composition.

The presently disclosed vaccine compositions can be administered in an effective amount in order to reduce the mammalian transmission of S. pneumoniae or to reduce the incidence rates of an invasive disease caused by S. pneumoniae. In certain embodiments, an “effective amount” of a vaccine composition can be an amount sufficient to achieve the desired result (i.e., reduced mammalian transmission of S. pneumoniae or to reduce the incidence rates of an invasive disease caused by S. pneumoniae). The vaccine compositions can be administered to a subject prior to infection by S. pneumoniae or prior to an invasive disease caused by S. pneumoniae or can be administered to a subject that has been infected by S. pneumoniae or that has an invasive disease caused by S. pneumoniae or is exhibiting symptoms of the same.

Generally, the presently disclosed compositions are administered in order to reduce the transmission of S. pneumoniae to other subjects within a population, but in those embodiments wherein the vaccine composition also comprises an additional S. pneumoniae immunogen that when targeted prevents colonization of the bacteria, the vaccine composition also serves to prevent colonization or to reduce the duration of colonization and functions prophylactically, and even therapeutically, in the subject to which it has been administered to prevent colonization and subsequent invasive disease within the vaccinated subject. In some of these embodiments, the vaccine compositions confer protective immunity, allowing a vaccinated individual to exhibit delayed onset of symptoms or reduced severity of symptoms, as the result of his or her exposure to the vaccine. In certain embodiments, the reduction in severity of symptoms is at least 25%, 40%, 50%, 60%, 70%, 80% or even 90%. In particular embodiments, vaccinated individuals may display no symptoms upon contact with S. pneumoniae, do not become colonized by S. pneumoniae, or both.

The specific effective dose level for any particular subject will depend upon a variety of factors including the specific composition employed; the age, body weight, general health, sex and diet of the patient; the time of administration; the route of administration; the rate of excretion of the composition employed; the duration of the treatment; drugs used in combination or coincidental with the specific compound employed; and like factors well known in the medical arts (see e.g., Koda-Kimble et al., (2004), Applied Therapeutics: The Clinical Use of Drugs, Lippincott Williams & Wilkins, ISBN 0781748453; Winter, (2003), Basic Clinical Pharmacokinetics, 4.sup.th ed., Lippincott Williams & Wilkins, ISBN 0781741475; Sharqel, (2004), Applied Biopharmaceutics & Pharmacokinetics, McGraw-Hill/Appleton & Lange, ISBN 0071375503). If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Administration of compositions described herein can occur as a single event, a periodic event, or over a time course of treatment. For example, agents can be administered daily, weekly, bi-weekly, or monthly. As another example, agents can be administered in multiple treatment sessions, such as 2 weeks on, 2 weeks off, and then repeated twice; or every 3rd day for 3 weeks.

5. Methods for Identifying Additional Genetic Factors Involved in Mammalian Transmission of S. pneumoniae

Methods are provided for identifying additional genetic factors involved in mammalian transmission of S. pneumoniae. The methods comprise infecting an influenza co-infected ferret with a ferret-transmissible strain of S. pneumoniae comprising a gene mutant library, and analyzing members of the gene mutant library that are able to colonize the infected ferret but not able to transmit or had a reduced transmission rate to contact ferrets.

Any ferret-transmissible strain of S. pneumoniae may be used in the presently disclosed methods. In certain embodiments, the ferret-transmissible strain of S. pneumoniae comprises serotype 19F strain BHN97.

Any mode of administration may be used to administer the S. pneumoniae comprising the gene mutant library to the ferret, although in some embodiments, the S. pneumoniae is administered to the ferret intranasally.

As used herein, a “gene mutant library” refers to a population of organisms in which, collectively within the members of the library, each non-essential gene within the genome of the organism has been mutated. The mutations can be introduced via any method known in the art for making genetic mutations, such as site-directed mutagenesis or randomized mutagenesis. In some embodiments, the gene mutant library comprises a transposon insertion library or transposon sequence (Tn-seq) library generated using transposon insertional mutagenesis. See, for example, Carter et al. (2014) Cell Host Microbe 15:587-599; Mann et al. (2012) PLoS Pathog 8:e1002788; van Opijnen et al. (2016) PLoS Pathog 12:e1005869; Verhagen et al. (2014) PLoS One 9:e89541; each of which is incorporated herein by its entirety.

Seasonal patterns of pneumococcal disease and colonization patterns support a role of respiratory viruses in promoting pneumococcal transmission, particularly the Influenza A virus (Althouse et al. (2017) Epidemiol Infect 145:2750-2758; Grijalva et al. (2014) Clin Infect Dis 58:1369-1376). Therefore, in particular embodiments, the ferrets are co-infected with an influenza virus. In some of these embodiments, the influenza virus comprises an influenza A virus. In particular embodiments, the influenza virus comprises Influenza/A/5/97 strain (H3N2). In certain embodiments, the ferrets are infected intranasally with influenza virus. In some embodiments, the ferret is infected with influenza prior to infection with S. pneumoniae. In some of these embodiments, the ferret is infected with influenza at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 1.5 days, 2 days, 2.5 days, 3 days, 3.5 days, 4 days or more before infection with S. pneumoniae. In particular embodiments, the ferret is infected with influenza about three days prior to infection with the ferret-transmissible strain of S. pneumoniae.

Contact ferrets are those ferrets that have been put into close physical contact with the infected ferret (i.e., placed within the same cage). The bacterial burden of the donor ferret (infected ferret) and contact ferrets can be assessed via induced sneezing and/or nasal lavage collection.

Analyzing members of the gene mutant library that are able to colonize the infected ferret but not able to transmit or had a reduced transmission rate to contact ferrets can be performed using any method known in the art of sequencing, including Illumina sequencing (van Opijnen (2015) Curr Protoc Microbiol 36, 1E 3 1-24; which is incorporated by reference herein in its entirety)

Identifying those members of the gene mutant library that were not able to transmit or had a reduced transmission rate to contact ferrets can be performed using any method known in the art, including using those described in the examples.

Once the mutated gene associated with a reduced transmission rate is identified, the identified genetic factor can be deleted or mutated in a murine-transmissible strain of S. pneumoniae, such as S. pneumoniae serotype 19F strain BHN97, followed by infection of a mouse with the mutated S. pneumoniae. Following infection, the transmissibility of the mutated S. pneumoniae to contact mice can be analyzed using similar methods as those described for the ferret model.

### EMBODIMENTS

1. A vaccine composition comprising at least one immunogenic polypeptide comprising at least one Streptococcus pneumoniae (S. pneumoniae) protein having an amino acid sequence set forth as any one of SEQ ID NOs: 1-205 or an immunogenic fragment or variant of any thereof, and a non-naturally occurring pharmaceutically acceptable carrier.

2. The vaccine composition of embodiment 1, wherein said S. pneumoniae protein is naturally expressed on the surface of S. pneumoniae.

3. The vaccine composition of embodiment 2, wherein said immunogenic polypeptide lacks a transmembrane domain.

4. The vaccine composition of embodiment 1, wherein said S. pneumoniae protein is naturally expressed on the surface of S. pneumoniae when S. pneumoniae is undergoing autolysis.

5. The vaccine composition of any one of embodiments 1-4, wherein said S. pneumoniae protein is conserved among two or more sequenced strains of S. pneumoniae.

6. The vaccine composition of embodiment 1, wherein said S. pneumoniae protein comprises at least one choline binding protein or an immunogenic fragment or variant thereof.

7. The vaccine composition of embodiment 6, wherein said S. pneumoniae protein comprises at least one choline binding protein having an amino acid sequence selected from the group consisting of SEQ ID NO: 27, 39, and 82; or an immunogenic fragment or variant of any thereof.

8. The vaccine composition of embodiment 1, wherein said S. pneumoniae protein comprises at least one protein selected from the group consisting of the sensor kinase of the competence cascade (ComD), the homolog of putative C3-degrading protease (CppA), and the iron transporter PiaA, or an immunogenic fragment or variant of any thereof.

9. The vaccine composition of embodiment 8, wherein said S. pneumoniae protein comprises at least one protein having an amino acid sequence selected from the group consisting of SEQ ID NOs: 10, 44, and 92, or an immunogenic fragment or variant of any thereof.

10. The vaccine composition of embodiment 8 or 9, wherein said S. pneumoniae protein comprises at least one of CppA and PiaA.

11. The vaccine composition of any one of embodiments 1-10, wherein said immunogenic polypeptide further comprises an additional pneumococcal immunogen.

12. The vaccine composition of any one of embodiments 1-10, further comprising an additional pneumococcal immunogen.

13. The vaccine composition of any one of embodiments 1-12, further comprising an immunological adjuvant.

14. The vaccine composition of any one of embodiments 1-13, wherein said composition is formulated for intranasal administration.

15. A method for reducing the mammalian transmission of Streptococcus pneumoniae (S. pneumoniae) by administering to a mammalian subject infected with S. pneumoniae or at risk of infection by S. pneumoniae a vaccine composition of any one of embodiments 1-14.

16. The method of embodiment 15, wherein said vaccine composition is administered to said mammalian subject intranasally.

17. The method of embodiment 15 or 16, wherein said method reduces the transmission of S. pneumoniae from a mother to its offspring.

18. A method for reducing the incidence rate of at least one invasive disease caused by Streptococcus pneumoniae (S. pneumoniae) in a mammalian population by administering to at least one mammalian subject within said mammalian population a vaccine composition of any one of embodiments 1-14.

19. The method of embodiment 18, wherein said vaccine composition is administered to said mammalian subject intranasally.

20. The method of embodiment 18 or 19, wherein said method reduces the transmission of S. pneumoniae from a mother to its offspring.

21. The method of any one of embodiments 18-20, wherein said at least one invasive disease is selected from the group consisting of pneumonia, acute otitis media, sepsis, meningitis, and bacteremia.

22. A method for identifying genetic factors involved in mammalian transmission of Streptococcus pneumoniae (S. pneumoniae), wherein said method comprises infecting an influenza co-infected ferret with a ferret-transmissible strain of S. pneumoniae comprising a gene mutant library, and analyzing members of said gene mutant library that are able to colonize said infected ferret but not able to transmit or had a reduced transmission rate to contact ferrets to identify genetic factors involved in mammalian transmission.

23. The method of embodiment 22, wherein said gene mutant library comprises a transposon sequencing (Tn-seq) library.

24. The method of embodiment 22 or 23, wherein said ferret-transmissable strain of S. pneumoniae is administered to said ferret intranasally.

25. The method of any one of embodiments 22-24, wherein said ferret-transmissible strain of S. pneumoniae comprises serotype 19F strain BHN97.

26. The method of any one of embodiments 22-25, wherein said influenza co-infected ferret is co-infected with Influenza/A/5/97 (H3N2).

27. The method of any one of embodiments 22-26, wherein said influenza co-infected ferret is co-infected intranasally with influenza three days prior to infection with said ferret-transmissible strain of S. pneumoniae.

28. The method of any one of embodiments 22-27, wherein said method further comprises deletion or mutation of said identified genetic factor in a murine-transmissible strain of S. pneumoniae, infection of a mouse with said murine-transmissible strain of S. pneumoniae, and analyzing transmissibility of said murine-transmissible S. pneumoniae to contact mice.

29. A method for reducing the mammalian transmissibility of Streptococcus pneumoniae by reducing the levels or activity of a protein having an amino acid selected from SEQ ID NOs: 1-205.

30. A method for reducing the mammalian transmissibility of Streptococcus pneumoniae by increasing the levels or activity of a protein that decreases tolerance of desiccation stress.

31. The method of embodiment 30, wherein said protein comprises a spxB protein or a spxR protein.

32. The method of embodiment 31, wherein said spxB protein comprises the amino acid sequence set forth as SEQ ID NO: 228 or said spxR protein comprises the amino acid sequence set forth as SEQ ID NO: 229.

33. The vaccine composition of any one of embodiments 1-14 for use as a medicament.

34. The vaccine composition for use according to embodiment 33, wherein said medicament is used to reduce the transmission of Streptococcus pneumoniae.

35. The vaccine composition of any one of embodiments 1-14 for use in reducing the transmission of Streptococcus pneumoniae.

### EXPERIMENTAL

### Example 1. Bacterial Factors Required for Transmission of Streptococcus pneumoniae in Mammalian Hosts

The first genetic screen for transmission factors in S. pneumoniae was performed by leveraging a highly saturated transposon sequencing (Tn-Seq) library of >6500 unique inserts in a ferret transmissible strain of pneumococci using influenza virus co-infected ferrets to recover enough unique transposon inserts from donor and contact ferrets to sufficiently power statistical predictions of the contribution of pneumococcal genes to transmission. These data revealed critical metabolic and regulatory cues that facilitate bacterial transmission as well as validated vaccine antigens designed to specifically inhibit bacterial transmission.

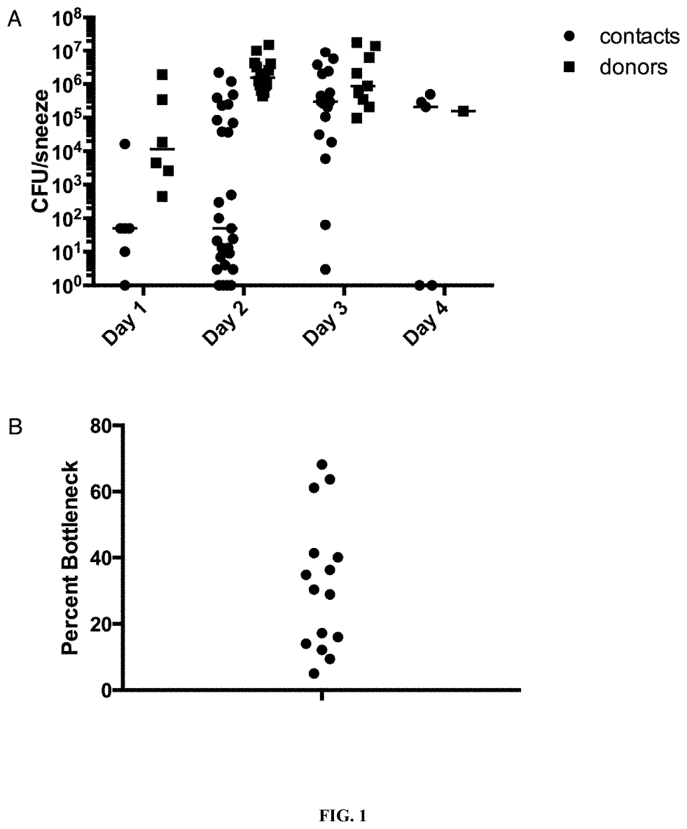

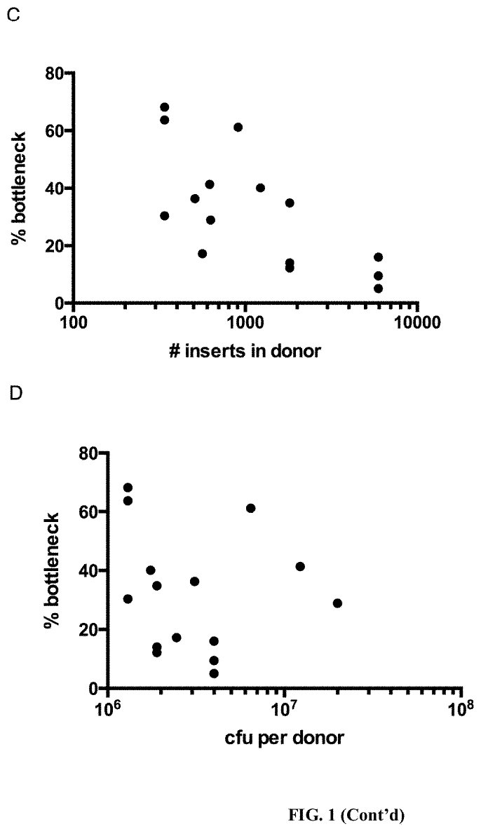

To enable the most permissive pneumococcal population bottlenecks, both donor and contact ferrets were intranasally infected with A/Sydney/5/97 (H3N2) influenza virus as previously described (McCullers et al. (2010) J Infect Dis 202:1287-1295), a strain and dosage designed for maximal recovery of pneumococcal populations from both donor and recipient animals. Three days following influenza virus challenge, one ferret per cage (donor) was infected with a TnSeq library generated in transmissible serotype 19F strain BHN97. Bacterial burden in donors and cagemates (contacts) was determined by daily induced sneezing and nasal lavage collection. Pneumococcal transmission was rapid and robust (FIG. 1A). Over 80% of contact ferrets were infected within 24 hours, and all contact ferrets were infected by three days post infection with greater than 85% of contact ferrets reaching a bacterial density of greater than 10⁴ CFU/sneeze. Total bacterial populations in nasal discharge were collected daily for four days, and total nasal bacterial population was collected by retrotracheal lavage at the end of the study. Input and total recovered bacterial populations from both donor and contact animals were prepared for Illumina sequencing as previously described (van Opijnen et al. (2015) Curr Protoc Microbiol 36,1E 3:1-24) and mapped to a high-quality closed genome of BHN97 (NCBI Accession No. PRJNA420094). It should be noted that insertion mutants that failed to initially colonize were eliminated from this analysis as the initial colonization event is a prerequisite for subsequent transmission. Comparison of output libraries of unique inserts from donor ferrets to contact ferrets identified a transmission bottleneck where between 5 and 68 percent of inserts in the donor animal are found in the contacts, with between 103-953 unique inserts present in contact ferrets (FIG. 1B). The transmission bottlenecks varied based on donor transposon and bacterial loads (FIGS. 1C and D). Comparison between the co-housed contacts indicated they share approximately 30% of inserts. These data indicate sufficient transmission was occurring for prediction of S. pneumoniae genes that are required for transmission between hosts.

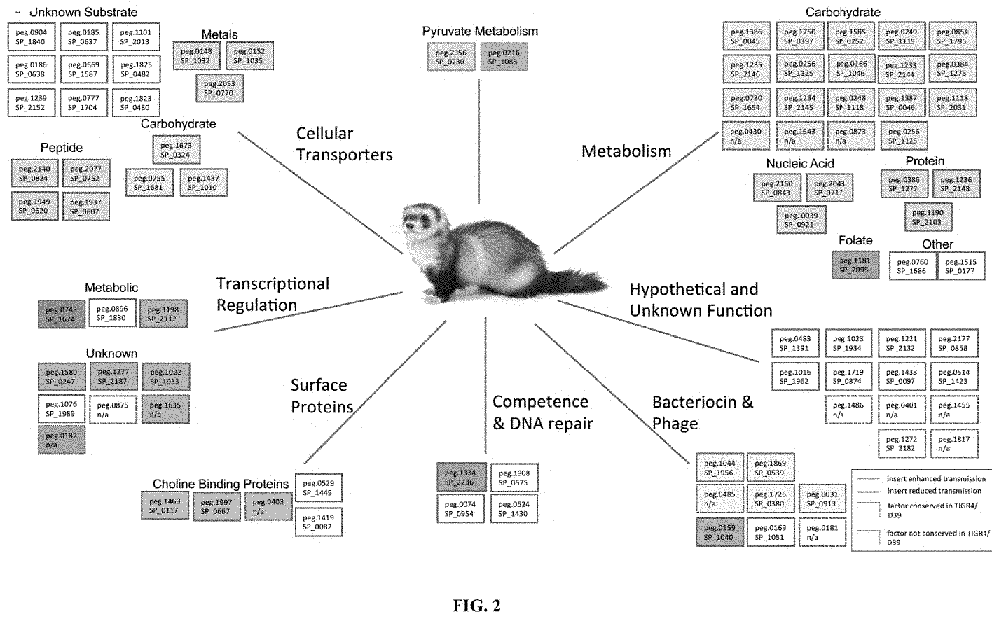

These data were evaluated to identify insertion mutants that were able to colonize the donor ferrets but were rarely or not recovered from contact ferrets. For each animal, the abundance of each mutant strain was quantified by counting the number of corresponding reads at each transposon insert site per gene obtained by next-generation sequencing. For contact animals, the read counts were dichotomized indicating whether the animal was infected (read count >10) or not infected (read count <10) by each strain. A cutoff of 10 reads was used because OTUs with zero counts in the input had up to 10 counts in the donors. Thus, it was recognized that spurious read counts of up to 10 were possible. 87 factors were identified (Table 1 and FIG. 2), 85% of which are conserved amongst the model strains of pneumococcus TIGR4 and D39, that while able to colonize at least 5 donors to high levels, were absent from all contact animals (Table 1). Using the density of colonization at each gene in the donor, a model was built to predict transmission based on colonization density. The model was used to compare the actual recovery from the contact animals to the modeled predictions to identify an additional 118 genes as significantly reduced (estimated transmission probability <10%) but not absent in recipient animals (Table 2). Many putative transmission factors were found to be involved in metabolism or metabolite transport (FIG. 2), suggesting metabolic sensing is key for transmission. Other classes of genes identified were regulatory, including four two-component systems: ComD (SP_2236), the sensor kinase of the competence cascade, SP_0662 whose cognate response regulator has been suggested to control expression of pilus (Basset et al. (2017) J Bacteriol 199:e00078-17), both SP_2000 and SP_2001, and response regulator SP_0156, which have not been implicated in other cellular processes. Additional transcriptional regulators were identified, many controlling expression of metabolic genes, further supporting a role for metabolic sensing and control during transmission. Three choline binding proteins were identified in the class of factors absent in all contact animals, including a choline binding protein (gene id=peg.403) that is encoded rarely in nasopharyngeal isolates, but is notably absent from laboratory strains TIGR4 and D39. These and other surface exposed factors could serve as adhesins or release factors during inter-host transmission. In addition to gene deletions with defects in transmission, two mutants over-represented in contact animals were identified, being successfully transmitted to 100% of the recipient animals despite not being overrepresented in the donors, suggesting deletion of these factors promote transmission. These factors were a known virulence factor, the pyruvate oxidase spxB (SP_0730) (Echlin et al. (2016) PLoS Pathog. 12(10), e1005951, published online Oct. 21, 2016, DOI: 10.137/journal.ppat.1005951; Pericone et al. (2000) Infect Immun. 68(7):3990-3997; Spellerberg et al. (1996) Mol Microbiol. 19(4):803-813) and its positive regulator, spxR (SP_1038) (Ramos-Montanez et al. (2008) Mol Microbiol 67:729-745).

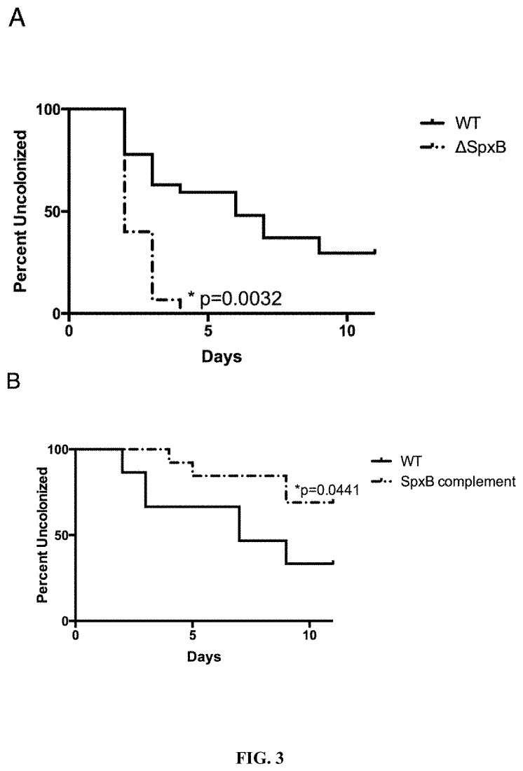

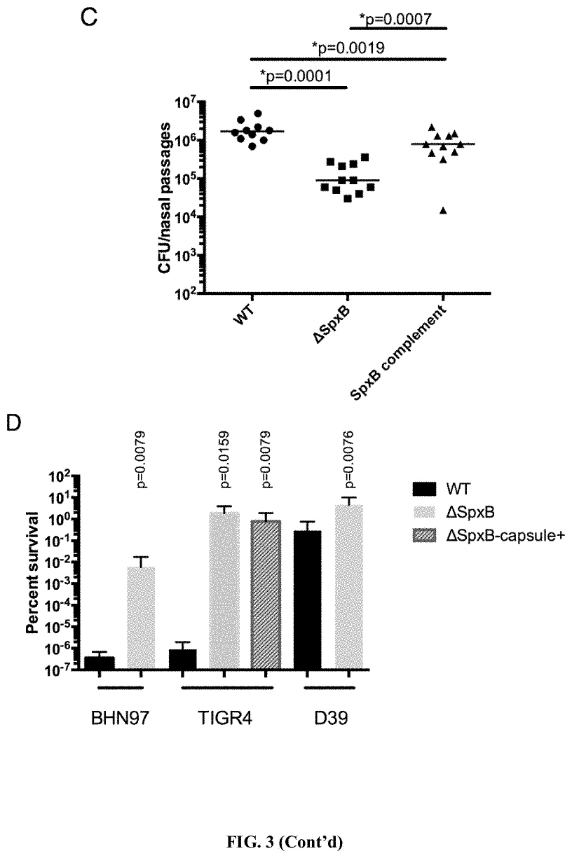

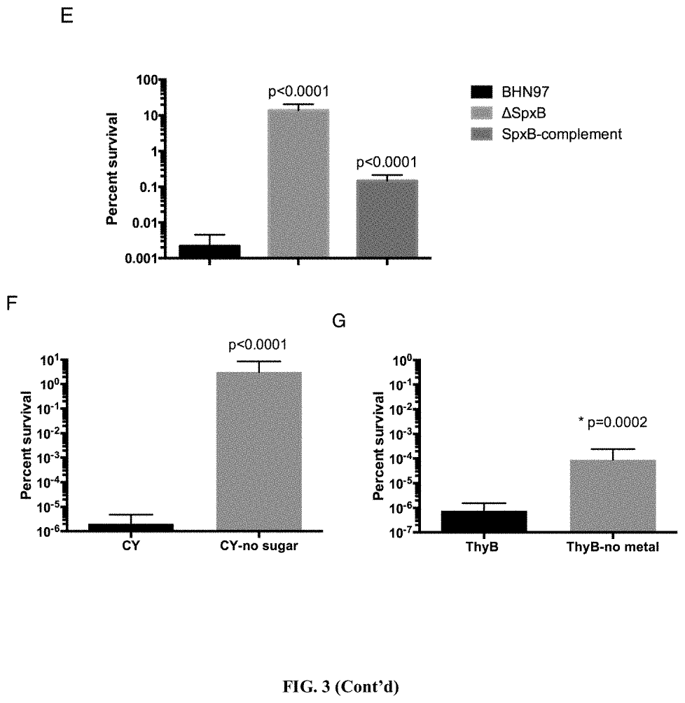

To confirm the Tn-seq predictions of gene deletions with altered transmission, targeted mutations in the transmissible strain background were generated and tested in the infant mouse model (Zafar et al. (2016)). Four day old pups were infected in a 1:1 donor to contact ratio and were sampled daily for ten days by taping the nares of the pup on an agar plate to identify bacteria present in the anterior nares. Pups were deemed colonized when bacteria were present on two consecutive sampling days. Confirmation of pneumococci was confirmed by random serotyping of the recovered colonies. Entire nasal passages were collected from donor and contact pups ten days post infection to enumerate colonization burden. The wild type strain, BHN97, is able to transmit in the absence of influenza co-infection, with 75-80% of contact pups becoming colonized within 10 days and 50% colonized by day 5 (FIG. 3A). The AspxB strain is able to transmit to 100% of contact pups, with 50% being colonized on day 2, highlighting a significant enhancement in transmission kinetics (FIG. 3A), even though this mutant is defective in colonization of donor animals (FIG. 3C). Complementation of SpxB resulted in reduction of transmission (FIG. 3B) and enhanced colonization of donor animals compared to the deletion strain (FIG. 3C). Identification of spxB was unexpected, as this virulence factor is best known for its contribution to the production of millimolar quantities of hydrogen peroxide generated by the pneumococcus and it would be expected that the remaining mutants with a functional copy of spxB would rescue the phenotype of the mutant if secreted hydrogen peroxide was the primary factor underlying altered transmissibility. It was hypothesized that additional cellular consequences arising from loss of SpxB activity were responsible for the heightened transmission phenotype.

Transmission can be contact dependent or airborne, requiring the bacteria to be outside of the host, either briefly as it transmits through the air, or for more prolonged periods of time in the environment. This study showed evidence of airborne transmission, as transposon inserts were present in contacts that were not present in cagemate donor animals, however were present in another donor in the room. However, the relative contributions of airborne versus direct nose-to-nose contact transmission between cagemate ferrets were unable to be determined. S. pneumoniae is capable of survival for prolonged periods in the extracellular environment following dehydration (Walsh and Camilli (2011) MBio 2:e00092-00011). It was hypothesized that both the reduced hydrogen peroxide production and other cellular consequences of glycolytic metabolic alterations by the spxB mutant displaying heightened transmissibility may impart fitness benefits during dehydration stress. Twenty-four hours post desiccation the AspxB strain displayed dramatically increased environmental stability via retention of viability when compared to the parental wild-type (FIG. 3D) with complementation of SpxB partially restoring desiccation sensitivity (FIG. 3E). The fitness benefit of the spxB deletion was independent of capsule type and strain background with similar phenotypes being observed in the serotype 4 TIGR4 and serotype 2 D39 strain backgrounds (FIG. 3D). Tolerance of desiccation stress was not related to metabolically induced alterations in capsule production (Echlin et al. (2016) PLoS Pathog 12:e1005951), as both encapsulated and non-encapsulated TIGR4 spxB mutants displayed similar desiccation stress phenotypes (FIG. 3D). The enzymatic reaction carried out by SpxB requires the glycolytic product pyruvate for its metabolic activity. As such, a similar desiccation tolerant phenotype might be expected to be imparted by nutrient starvation conditions. Upon shifting cultures of S. pneumoniae into media deficient of a carbon source, a significant desiccation tolerance phenotype was conferred to the wild type cultures (FIG. 3F).

Carbon source limitation is not the only means by which bacterial pathogens are metabolically constrained in the mammalian host. Transition metal bioavailability is also a critical aspect of successful pneumococcal colonization (Turner et al. (2017) Adv Microb Physiol 70:123-191) with both bacterial and host (Palmer et al. (2016) Annu Rev Genet 50:67-91) strategies for metal acquisition and sequestration, respectively. It was hypothesized that metal limitation would impart a similar desiccation tolerance phenotype to S. pneumoniae due to the reduced metabolic activity under such metal starvation conditions. Upon transfer to metal-depleted media conditions, a similar phenotype was observed to that of carbohydrate deprived cells, with a significant increase in desiccation tolerance being observed in cells cultured under metal-limiting conditions compared to cells cultured in standard non-depleted media (FIG. 3G). Taken together these data indicate that nutrient limiting conditions significantly increase the capacity of the pneumococcus to survive desiccation stress and in turn promote both environmental stability and transmissibility of S. pneumoniae.

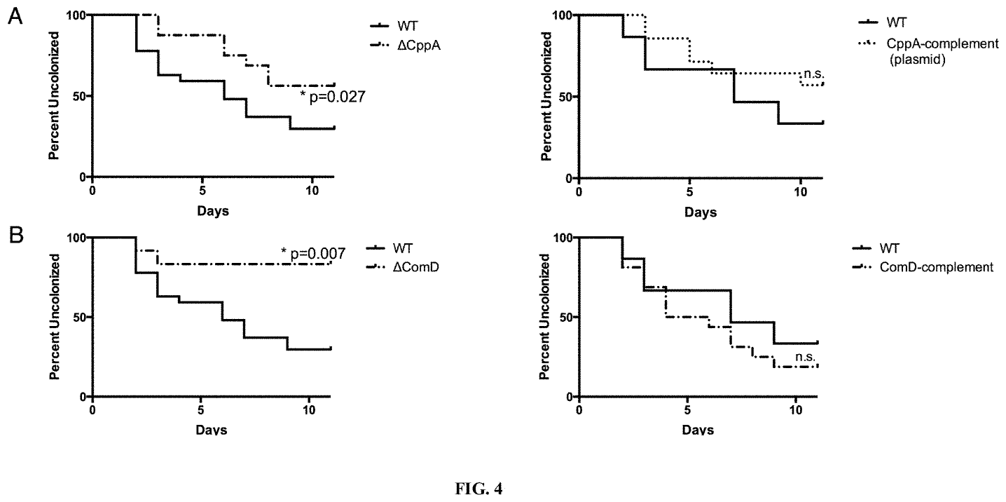

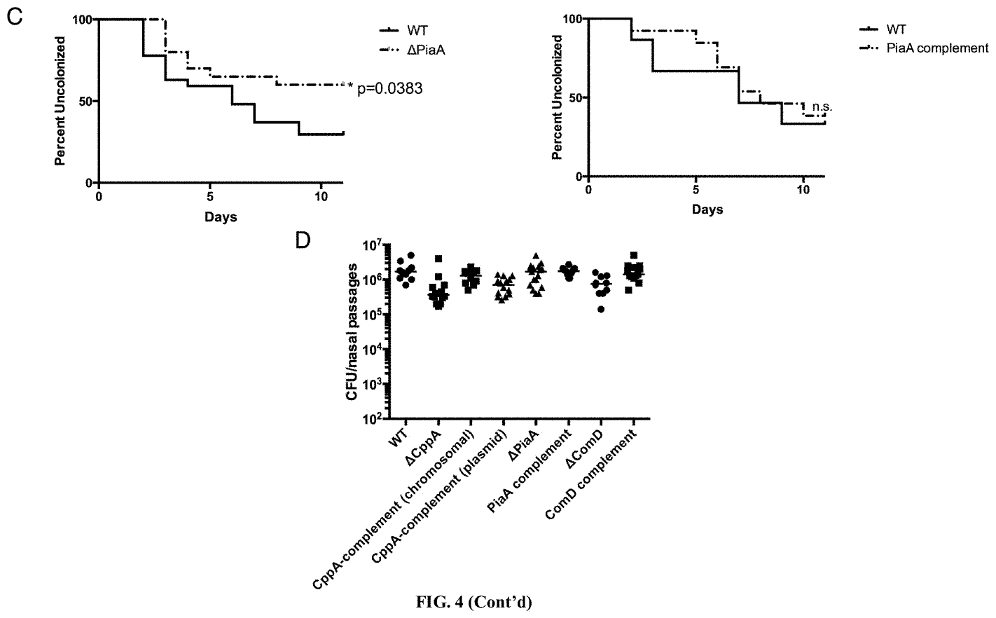

To confirm the factors required for transmission identified in the ferret screen, targeted deletions were again made in the transmissible BHN97 strain and transmission dynamics tested in the infant mouse model. Deletion of homolog of putative C3-degrading protease SP_1449 (CppA), iron transporter PiaA (SP_1032 homolog), or competence regulatory histidine kinase ComD (SP_2236 homolog), significantly decreased transmissibility from 75-80% by wild type BHN97 to 50, 45 and 13% respectively (FIG. 4A-C) despite similar colonization burdens in donor pups (FIG. 4D). Complementation of ComD and PiaA by insertion into an inert region of the chromosome, or CppA by overexpression on a plasmid, restored transmission levels to levels statistically indistinguishable from the parental wild-type (FIG. 4A-C). BlpM (SP_0539 homology) and LivG (SP_0752 homolog) were unable to be confirmed in the influenza-naïve pups. This could suggest these factors may only be important for transmission in the context of IAV co-infection in the context of the ferret respiratory tract, or in the context of adult animals, or a combination of such factors. These data generally confirm the Tn-seq predictions of the contribution of pneumococcal genes to mammalian transmission.

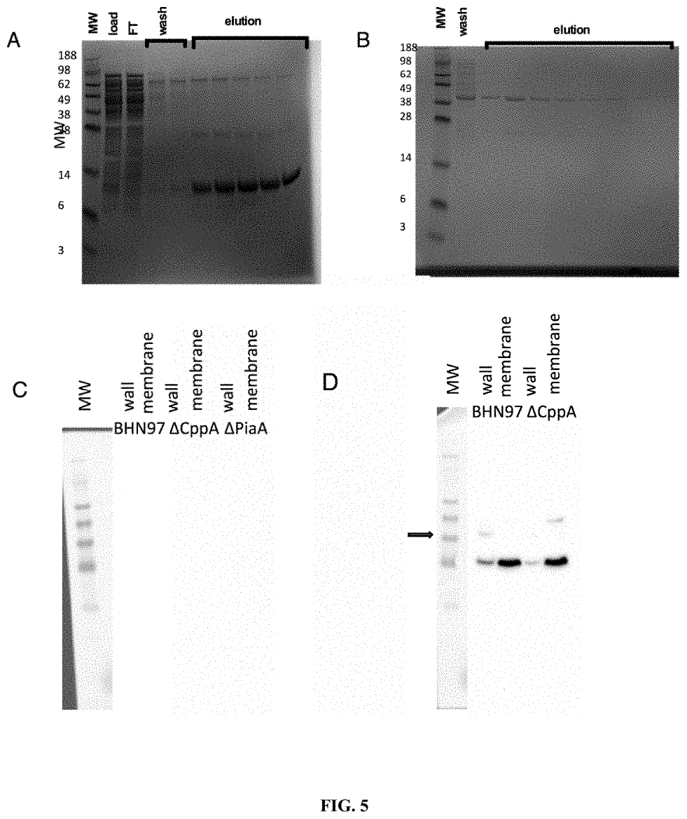

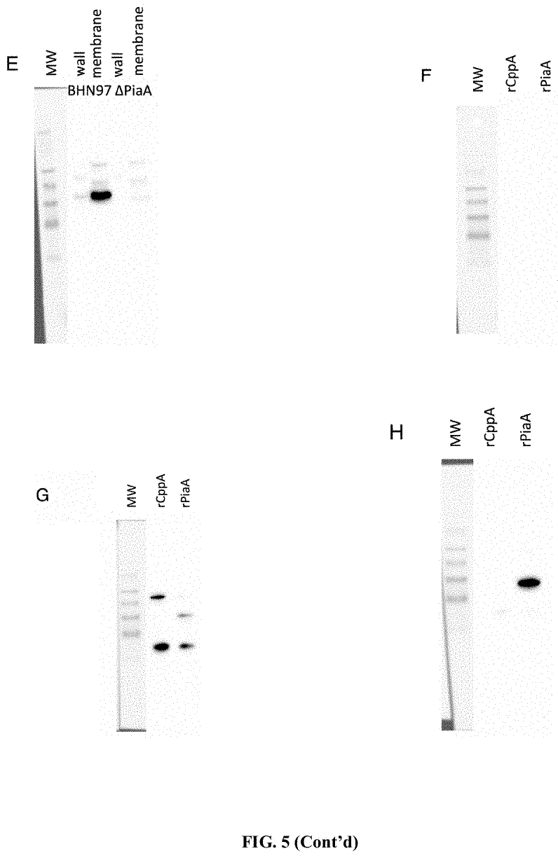

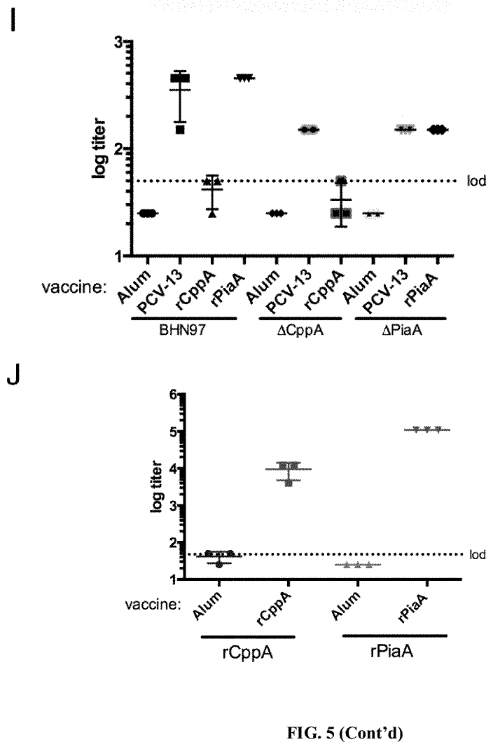

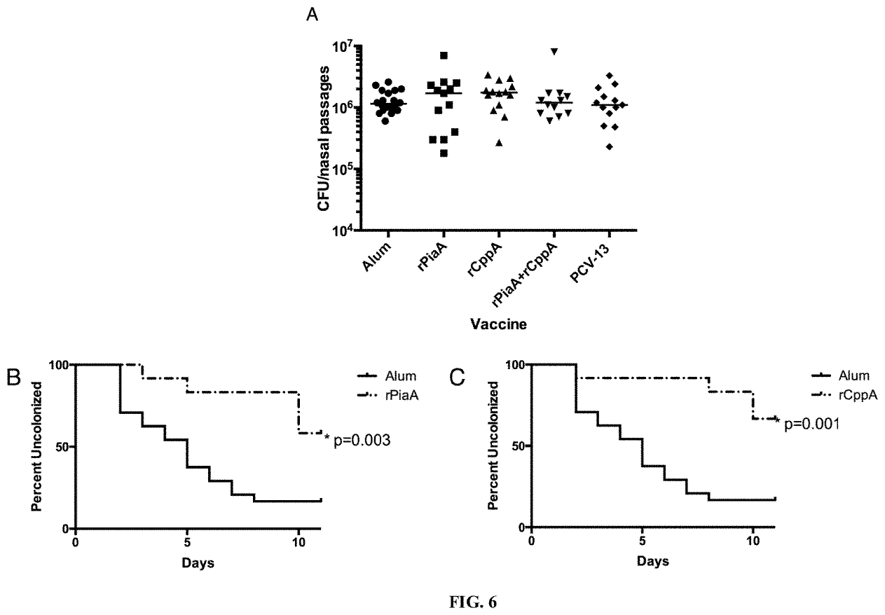

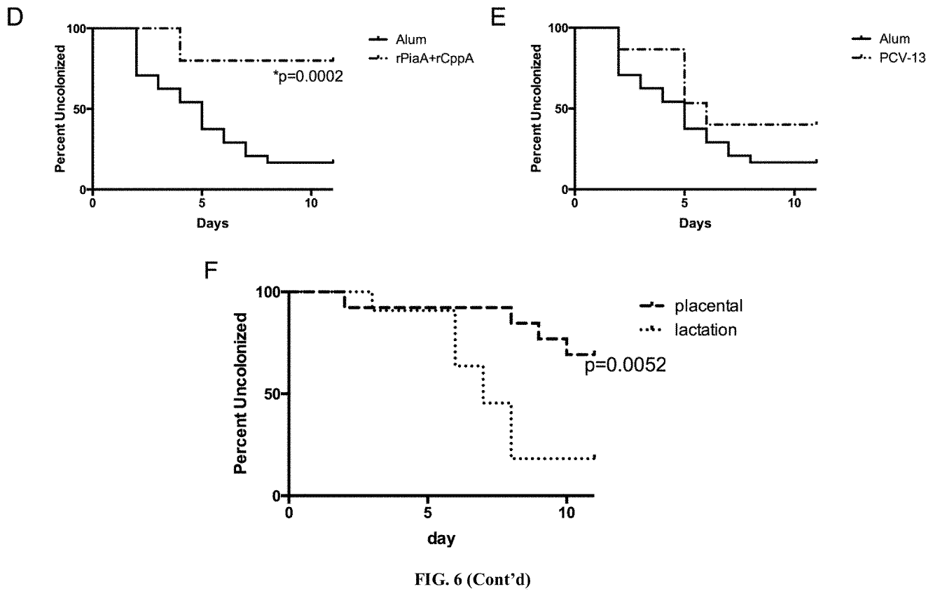

Capsule-based vaccines are extremely effective against invasive pneumococcal disease due to the requirement for capsule during systemic infection. Upon introduction of the conjugate vaccine, pneumococcal populations rapidly undergo a shift towards non-vaccine serotypes that continue to colonize at equivalent rates (Weinberger et al. (2011) Lancet. 378(9807):1962-1973). It was hypothesized that vaccination with antigens based on the pneumococcal factors required for transmission may result in effective inhibition of bacterial spread between hosts and hence represent a novel vaccination strategy for eliminating this opportunistic pathogen from the population. Recombinant forms of the transmission factors PiaA (Brown et al. (2001) Infect Immun. 69(11):6702-6706) and CppA (Carter et al. (2014) Cell Host Microbe. 15(5):587-599), both alone or in combination, were utilized to vaccinate female mice, which were subsequently allowed to breed. Both of these factors are highly conserved, with PiaA being conserved in a majority of available S. pneumoniae genomes, and CppA present in all publicly available pneumococcal genomes. Additionally, these antigens have been shown to be immunogenic in mice and protective against systemic challenge (Carter et al. (2014)). As controls, mice were also vaccinated with either alum control or the currently licensed PCV-13 vaccine. ELISAs and Western Blots indicated that vaccination induced antibody responses against both antigens (FIG. 5). Neonatal mice from these respective litters were utilized in the murine transmission model with half the pups receiving S. pneumoniae and the other half marked as recipient animals. Vaccination with any of these antigens conferred no significant protection benefit during the initial colonization of the donor animals (FIG. 6A). A high degree of protection from pneumococcal transmission was engendered by vaccination with either PiaA (FIG. 6B) or CppA (FIG. 6C), or using both proteins (FIG. 6D), resulting in a significant delay and lower overall rates of transmission due to maternal vaccination. In contrast, no significant protection against transmission was observed in the pups from dams vaccinated with PCV-13 compared to alum controls (FIG. 6E). This was of particular interest as serotype 19F is included in the PCV-13 vaccine, indicating lack of efficacy even in cases of homologous challenge. Maternal vaccination can provide protection both through transplacental antibody trafficking and the presence of maternal antibody in milk. Cross-fostering experiments where pups of vaccinated dams were nursed by non-vaccinated dams, and pups of non-vaccinated dams were nursed by vaccinated dams suggest the protection primarily comes from transplacental antibody (FIG. 6F). Thus, vaccines specifically tailored to target factors involved in pneumococcal transmission may be a vital strategy for elimination of S. pneumoniae from populations in a serotype independent manner.

These data represent a comprehensive genetic screen to identify bacterial factors required for the mammalian transmission of S. pneumoniae. These data suggest that under conditions of metabolic limitation, the pneumococcus demonstrate heightened environmental stability. This may result in the bacteria becoming increasingly transmissible, a phenotype mimicked via deletion of the pyruvate oxidase SpxB. The advantage for transmissibility of loss of SpxB would suggest that this factor should have been lost to evolution. However, SpxB is also important for colonization, indicating that striking a balance between colonization and transmissibility is vital for pneumococcal biology. This screening methodology allowed for the identification of numerous pneumococcal genes that were required for successful transmission between mammalian hosts. The identification of ComD as a transmission factor complements findings of the importance of pneumococcal competence in colonization (Marks et al. (2010); Shen et al. (2019); Zheng et al. (2017)) in addition to its role in genetic exchange. Three two-component regulatory systems previously not implicated in transmission were also identified, as well as iron acquisition and complement evasion surface proteins. A number of hypothetical proteins of unknown function were also identified, indicating a number of important functional aspects of transmission remain to be characterized. Utilization of the surface exposed transmission factors as vaccine antigens proved extremely efficacious at preventing transmission between donor and contact animals independent of donor colonization burden. Rationally designed combination vaccines could prove to be especially effective at blocking both transmission and invasive disease. These data indicate that vaccines targeting transmission may prove an efficacious strategy for the elimination of S. pneumoniae from populations.

### Materials and Methods

### Ferret Transmission Factors Screen

Nine week old male castrated ferrets (Triple F Farms) housed three to four per cage were infected intranasally under 4% isoflurane sedation, with 10⁵ TCID₅₀/mL A/Sydney/5/1997 (H3N2) influenza virus in a volume of 1 mL PBS (0.5 mL per nostril) (McCullers et al. (2010) J Infect Dis 202:1287-1295). Three days post viral challenge, one ferret per cage (donor) was infected with 10⁷ CFU BHN97 transposon library, in a volume of 0.6 mL PBS (0.3 mL per nostril). Each day post bacterial infection, 1 mL nasal washes (0.5 mL per nostril of PBS) were collected following ketamine sedation of ferrets from donor and cage mate (contact) ferrets for four days, and additionally at day four, ferrets were euthanized and post terminal retro-tracheal lavage collected in 2 mL PBS. Aliquots were removed for bacterial and viral burden determination, and the remainder was plated on selective media.

### Neonatal Mouse Transmission

One male and one female adult C57BL/6 mice (Jackson Labs) were housed per cage. Four days after pups were born, all pups (both male and female) were toeclipped for identification, one half of the litter (the donors) was infected intranasally with 2000 CFU BHN97 or mutant strain in 3 μL PBS without sedation. The rest of the litter was designated contacts and was not infected. Each day for 10 days post infection of the donors, the nares of each pup were tapped 20 times on a TSA/blood agar plate supplemented with 20 μg/mL neomycin, and spread with a sterile loop for CFU enumeration. Following two consecutive positive samples, a contact pup was determined to be colonized. On day 10 post infection of donors, all pups were euthanized by CO2 asphyxiation followed by cervical dislocation. Heads were defleshed, bottom jaw and brain removed and entire skull was homogenized with a 5 mL syringe plunger and a 100 μm cell strainer. Sample was collected in 750 μL of PBS, diluted and plated for CFU enumeration.

### Maternal Vaccination

Adult female C57BL/6 mice (Jackson Labs) were vaccinated by intraperitoneal injection with 1:50 human dose PCV-13 or 10 μg recombinant protein: rCppA or rPiaA (prepared as described below) conjugated to 130 μg alum in a volume of 100 μL PBS two weeks prior to mating and then boosted every two weeks for the duration of the study. Pups were infected with wild type BHN97 and monitored as above. For analysis of circulating antibody response by western and ELISA, two additional adult male mice were vaccinated with each antigen and boosted twice. One week following the final boost, the mice were anesthetized with 4% isoflurane and bled by retro-orbital route and maximum blood volume was collected. Mice were then euthanized by CO2 asphyxiation followed by cervical dislocation.

### Bacterial Growth Conditions

Streptococcus pneumoniae strains were grown in ThyB or CY (see below) in static conditions at 37° C. +5% CO2 for liquid culture and on TSA/blood agar at 37° C. +5% CO2 for solid culture. TSA/blood agar plates were prepared from 40 mg/L tryptic soy agar (EMD Millipore, GranuCult, item number 105458) in distilled water, and then autoclaved for 45 minutes. After cooling to 55° C., 3% defibrinated sheep blood was added (Lampire biological, item number 7239001) and poured into 100×15 mm round petri dishes. Media was supplemented with 20 μg/mL neomycin for all animal derived samples to reduce contamination with environmental Staphylococci endemic to our animal colonies. Media for Tn-Seq samples were additionally supplemented with 200 μg/mL spectinomycin to select for transposon. Deletion mutants were selected on TSA/blood agar with 1 μg/mL erythromycin. Chromosomal complementation mutants were selected on TSA/blood agar supplemented with 1 μg/mL erythromycin and 150 μg/mL spectinomycin. Plasmid complementation of CppA was selected on TSA/blood agar supplemented with 1 μg/mL erythromycin and 400 μg/mL kanamycin.

ThyB was prepared from 30 g/L Todd Hewitt (BD item number 249240) with 2 g/L yeast extract (BD item number 212750) in distilled water and autoclaved 45 minutes.

ThyB metal deplete was made by mixing ThyB with 15 g Chelex resin (BioRad item number 14201253) overnight followed by filtration to remove resin.

CY media was prepared as follows:

Supplement: Prepare the supplement “3 in 1” salts by adding 50 g MgCl₂ 6H2O, 0.25 g CalCl2 anhydrous and 0.1 mL (0.1 M) Manganese sulfate 4 H₂O in 500 ml distilled water (dH2O), mix well, and autoclave. Prepare 20% Glucose, 50% Sucrose, 2 mg/ml Adenosine and 2 mg/ml Uridine 500 ml each respectively, and filter sterilize. Combine all 5 components at the following ratio: 60 ml “3 in 1” salts, 120 ml 20% Glucose, 6 ml 50% Sucrose, 120 ml 2 mg/ml Adenosine, 120 ml 2 mg/ml Uridine, mix in a beaker and filter sterilize, label as Supplement, store at 4° C.

Adams Solutions: Prepare Adams I by combining the following chemicals: 30 mg Nicotinic Acid (Niacin stored at 4° C.), 35 mg Pyridoxine HCl (B6), 120 mg Ca-Pantothenate (stored at 4° C.), 32 mg Thiamine-HCl, 14 mg Riboflavin, and 0.06 ml Biotin (0.5 mg/ml stock). Add dH2O to 200 ml, then add 1-5 drops of 10N NaOH to dissolve chemicals, filter sterilize and store in foiled bottles at 4° C. Prepare Adams II by adding the following chemicals: 50 mg FeSO47H2O, 50 mg CuSO4, 50 mgZnSO47H2O, 20 mg MnCl2 and lml HCl, up to 100 ml dH2O, filter to sterilize, store at 4° C. Prepare Adams III by adding the following 5 components: 800 mg Asparagine, 80 mg Choline Chloride, 64 ml Adams I, 16 ml Adams II and 0.64 ml CaCl2 (1% stock), to 400 ml d H2O. Filter sterilize solutions and store in foiled bottle at 4° C.

Buffers: Prepare 1M KH2PO4 and 1M K2HPO4 (autoclaved) as the stocks, mix 26.5 ml 1M KH2PO4 and 473 1 M K2HPO4 and stir well, do not titrate, filter to sterilize, 4° C.

PreC: Prepare PreC by mixing the following chemicals: 4.83 g Sodium Acetate (Anhydrous), 20 g Difco Casamino Acids/technical, 20 mg/L-Tryptophan, and 200 g/L Cysteine HCl, dissolve in 800 d H2O, adjust pH to 7.4-7.6 by adding 10 N NaOH, stir well for 60 minutes, fill up to 4 liter dH2O, mix well, aliquot 400 ml portions in 500 ml flasks, autoclave for 30 minutes, and store at 4° C.

C+Y: Add 0.5 g glutamine to 500 ml dH2O, filter to sterilize and store at 4° C. Add 2 g pyruvic acid (stored at 4° C.) to 100 ml dH2O, filter to sterilize, and store at 4° C. Solve 5 g yeast to 100 ml dH2O (25 g in 500 ml), and autoclave (filter to sterilize if necessary). Add 6 of the following solutions to 400 ml PreC: 13 ml Supplement, 10 ml Glutamine, 10 ml Adams III, 5 ml Pyruvate, 15 ml K-Phosphate buffer, and 9 ml Yeast. Filter sterilize and store at 4° C. CY sugar deplete media was made as above but with the omission of glucose, sucrose and yeast extract.

### Construction of Mutants in BHN97

Allelic replacement deletion mutants were made by replacement of the gene of interest with an erythromycin cassette by splicing by overlap extension PCR. A region 1.5-2 kb upstream and downstream of the gene of interest was amplified by PCR using Takara HotStart polymerase according to manufacturer instructions from BHN97 genomic DNA using the primers in Table 3 with an overhang corresponding to the beginning and/or end of the erythromycin cassette. The upstream and downstream fragments were mixed with the erythromycin cassette and amplified with Takara polymerase and upstream forward and downstream reverse primer. The resultant PCR product was gel purified using Qiagen MinElute kit (item number 28606) according to manufacturer's instructions. BHN97 was transformed with the purified PCR product in CY media using both CSP-1 and CSP-2.

Chromosomal complements were made by insertion of the gene and 150-200 bases upstream containing the promoter, followed by a spectinomycin cassette into a region downstream of amiF similar to as described in (Guiral et al. (2006) Microbiology 152(Pt 2):343-349) except a phage is inserted into the exact chromosomal region described therein in BHN97; therefore, insert regions were changed slightly, as to not disrupt the downstream gene. A region of approximately 1.5 kb downstream of amiF was amplified with primers BHN97 insert UP FWD and BHN97 insert DOWN-Spec (see Table 3) as described above, and then mixed with spectinomycin cassette and amplified. The gene plus upstream primer region was amplified with an overlap of the spectinomycin cassette at the 3′ end and an overlap of the region containing amiF at the 5′ end (see Table 3 for primers). The region containing amiF was amplified with primers BHN97 insert amiF FWD and BHN97 insert amiF REV (see Table 3). All three fragments were mixed and amplified with primers BHN97 insert amiF REV and BHN97 insert UP FWD. The resulting PCR product was gel purified and transformed into the deletion strain as described above. Except for complementation of comD, where due to deletion of comD the strain was no longer competent, and therefore the complementation construct was transformed into BHN97 and then the deletion construct was transformed in to the resultant strain.

Complementation of cppA transmission phenotype was not able to be accomplished via chromosomal complementation. Therefore an overexpression construct was made in pABG5 (Granok et al. (2000) J Bacteriol. 182(6):1529-1540) by amplification of cppA from the BHN97 genome using primers 5′ BHN97 CppA EcoRI and 3′ BHN97 CppA PstI and digested with EcoRI and PstI (NEB) according to manufacturer's instructions. pABG5 was also digested with EcoRI and PstI. Plasmid and insert were ligated overnight at 14° C. in a thermocycler and transformed into One Shot TOP10 E. coli according to manufacturer's instructions. Transformants were selected on LB agar (BD, BP1425-500) supplemented with 50 μg/mL kanamycin. Following confirmation by Sanger sequencing, the plasmid was transformed into the cppA deletion strain as described above. Maintenance of plasmid was ensured by addition of 400 μg/mL to any broth during culture of this strain. SpxB mutants in strain D39 and TIGR4 were previously constructed (Echlin et al. (2016) PLoS Pathog. 12(10):e1005951).

### Viral Culture

Influenza virus was grown in the allantoic fluid of 10-11 day embryonated chicken eggs and titered on MDCK (Manin Darby Canine Kidney) cells by infection with 100 μL 10-fold serial dilutions of sample and incubated at 37° C. for 72 hours. Following incubation, viral titers were determined by hemagglutination assay using 0.5% turkey red blood cells and analyzed by the method of Reed and Munch (Reed (1938) The American Journal of Hygiene 27:493-497).

Preparation of BHN97 TnSeq Library TnSeq library was prepared in strain BHN97 as previously described (van Opijnen et al. (2015) Curr Protoc Microbiol. 36:1E 3 1-24). Briefly, in vitro transposition was performed using purified BHN97 genomic DNA, plasmid pMagellan6 as source of transposon and purified MarC9 protein as transposase. DNA was purified by ethanol precipitation. Transposition junctions were repaired with T4 DNA polymerase (NEB) and E. coli DNA ligase (NEB). Ligated product was transformed into strain BHN97 as described above in construction of mutants. Transformations were plated on selective media and grown overnight at 37° C.+5% CO2. All growth was collected into ThyB media, and glycerol was added to a final concentration of 20%, and libraries were stored at −80° C. Six libraries were combined and expanded in 100 mL of ThyB until at midlog growth. Glycerol was added to a final concentration of 20% and 1 mL aliquots frozen for use in all experiments. For each ferret infection, one 1 mL aliquot was added to 9 mL ThyB and grown to midlog (OD₆₀₀=0.4).

### Preparation of Output Libraries

All of the sneezed material or retrotracheal lavage was plated on 3-5 plates of selective media and grown at 37° C. +5% CO2 overnight. 5 mL PBS was added to each plate and all growth resuspended and collected. Bacteria were pelleted by centrifugation, supernatants discarded and pellets stored at −80° C. Genomic DNA was extracted from bacterial pellets using the Blood and Tissue Kit (Qiagen) according to manufacturer's instructions for Gram-positive bacteria. Tn-Seq libraries were prepared as previously described (van Opijnen et al. (2009) Nat Methods. 6(10):767-772; van Opijnen and Camilli (2010) Curr Protoc Microbiol. Chapter 1, Unit1E 3). Briefly, genomic DNA was digested with MmeI (NEB) and cleaned up via standard phenol chloroform extraction. Then adapters were ligated onto the digested DNA and PCR amplified with Q5 polymerase (NEB). PCR products were gel extracted. Sequencing was performed on Illumina HiSeq platform.

### Desiccation Resistance of Bacterial Hyper-Transmission Mutants

Bacteria were grown in ThyB to midlog, and 1 mL aliquots transferred to 1.7 mL microcentrifuge tubes and centrifuged to remove all residual media. Tubes were spun open for 90 minutes in a SpeedVac until pellet was dry. Tubes were closed and stored in the dark at room temperature. 24 hours post desiccation, bacterial pellets were resuspended in 100 μL PBS and plated for viability. For desiccation resistance in sugar depleted media, pneumococci were grown in CY until midlog and then shifted to CY or CY lacking glucose, sucrose and yeast extract and allowed to grow for two hours, then desiccation protocol followed. Metal depleted media was made by depletion of ThyB using Chelex resin. Midlog bacteria grown in ThyB were shifted to metal deplete or replete media and exposed for two hours, followed by the desiccation protocol. All were done with at least 4 technical replicates, and three biological replicates. Percent survival was calculated for each technical replicate by dividing the post desiccation CFU/mL with the pre-desiccation CFU/mL of the culture and then multiplied by 100 to give percent survival. Desiccation resistance of the SpxB complemented strain was done using a different vacuum pump and desiccation proceeded more rapidly.

Expression and Purification of rCppA and rPiaA

Both rCppA and rPiaA were generated by the protein production facility at St. Jude Children's Research Hospital. PiaA was expressed and purified as described in (Carter et al. (2014) Cell Host Microbe. 15(5):587-599). The coding sequence for CppA was amplified from TIGR4 and cloned into the pET28b cloning vector in BL21-DE3 cells. Cultures were grown to OD600=0.5 and induced with 0.07 mM IPTG overnight at 23° C. Bacterial pellets were lysed with Bugbuster (Novogen) reagent according to manufacturer's protocols. CppA was purified on His-Selected Nickel Affinity Gel following manufacturer's protocol for native conditions. Protein was dialyzed using Pierce Slide-A-Lyzer dialysis cassette overnight with sterile PBS. Dialyzed protein was stored at −80° C. in a 10% glycerol solution until further use. PiaA was expressed and purified as previously described (Brown et al. (2001) Infect Immun. 69(11):6702-6706). High-level expression of His₆-PiaA was achieved by the addition of isopropyl-β-d-thiogalactoside (IPTG) to a final concentration of 2 mM, and the cultures were incubated for a further 4 h. The cells were harvested by centrifugation at 6,000×g for 10 min and resuspended in lysis buffer (50 mM sodium phosphate, pH 8.0; 2 M NaCl; 40 mM imidazole). The cells were lysed in a French pressure cell (SLM Aminco, Inc.) at 12,000 lb/in², and the lysates were centrifuged at 100,000×g for 1 h. Then, 20 mM β-mercaptoethanol was added to the resultant supernatants, which were loaded onto 2-ml nickel-nitrilotriacetic acid resin columns (ProBond; Invitrogen) previously equilibrated with five column volumes of lysis buffer. The columns were washed with 10 column volumes of 10 mM sodium phosphate, 20 mM imidazole, and 1 M NaCl (pH 6.0), and the proteins were eluted with a 30-ml gradient of 0 to 500 mM imidazole in 10 mM sodium phosphate (pH 6.0). Fractions of 3 ml were collected and analyzed by SDS-PAGE to identify fractions containing abundant purified protein. The selected fractions were dialyzed extensively against 10 mM sodium phosphate (pH 7.0) to remove the imidazole. The purified His₆-PiaA protein was then resuspended in 50 mM sodium phosphate (pH 7.0), glycerol was added to a final concentration of 50%, and the proteins stored at −15° C. Purity of protein was determined to be >95% by visualization on a 10% Bis-Tris gel stained with Simply Blue Safestain (Invitrogen).

### Fractionation

Sera from vaccinated animals were analyzed against cell wall and cell membrane fractions from BHN97, ΔCppA, and ΔPiaA.

Cell wall fraction was prepared by the following methods. A 100 mL culture of each bacteria were grown in ThyB to OD₆₂₀˜0.6. Bacteria were pelleted by centrifugation, washed in 50 mM Tris-HCl, 1 mM EDTA pH 8.0 supplemented with 1×HALT protease inhibitor (Thermo) and pelleted by centrifugation. Pellets were resuspended in 1 mL 50 mM Tris-HCl pH 8.1, 1 mM EDTA pH 8.1 plus 20% (w/v) sucrose, 10 mg/mL chicken egg white lysozyme and 250 U mutanolysin, and incubated 2 hours at 37° C. with shaking. Protoplasts were pelleted at 14,000×g for 10 minutes. Supernatant (cell wall fraction) was removed and stored at −20° C. Membranes were prepared by resuspending protoplasts (pellet from cell wall prep) in 10 mM Tris-HCl pH 8.1, 50 mM MgCl₂ and 10 mM glucose, supplemented with 1×HALT protease inhibitor and lysed with 0.1 mm zirconia/silica beads in a FastPrep 24 system (MP Biologicals) for 3, 20 second pulses with one minute rests on ice between pulses. Beads and intact cells were pelleted at 6000×g for 10 minutes. Supernatant containing membranes was transferred to ultracentrifuge tube and ultra-centrifuged 30 minutes at 45,000×g at 4° C. Supernatant containing cytoplasmic proteins was removed. Pellet was resuspended in 10 mM Tris-HCl pH 8.1, 20 mM MgCl₂ and 50 mM NaCl and ultra-centrifuged 45 minutes at 100,000×g at 4° C. Supernatant was discarded and pellet was resuspended in 100 μL 10 mM Tris-HCl pH 8.1, 20 mM MgCl₂ and 50 mM NaCl.

### Western Blot

100 μL sample was combined with 30 μL 4× sample buffer (NuPAGE, Invitrogen) and boiled 10 minutes. 15 μL was loaded into 15 well 4-12% Bis-Tris precast gel, and run 1 hour 45 minutes at 80V in NuPAGE running buffer. Gels were transferred to nitrocellulose membranes at 30V for 90 minutes in NuPAGE transfer buffer supplemented with 20% methanol. Membranes were blocked 1 hour at room temperature in 4% non-fat dry milk in PBS supplemented with 0.1% Tween-20 (PBST). Membranes were incubated with sera from vaccinated mice at a 1:5000 dilution in 4% non-fat dry milk in PBST overnight at 4° C. Blots were washed 3×10 minutes with PBST. Membranes were incubated with secondary antibody, goat anti-mouse IgG-HRP (Invitrogen), 1:5000 in 4% non-fat dry milk in PBST 3 hours at room temperature. Blots were washed 3×10 minutes in PBST and 1×5 minutes in PBS. 2 mL each SuperSignal West Dura Extended Duration Substrate (Thermo Scientific) reagent was added to each blot and incubated for 5 minutes at room temperature. Blots were imaged on a ChemiDoc MP (BioRad) using ImageLab 5.0 software, automatic exposure settings for Chemiluminescence, high specificity, optimizing for bright bands. Purified protein blots were performed as above except 500 ng each protein was used as sample.

### ELISA

Sera from vaccinated animals were analyzed by ELISA. Bacterial strains BHN97, ΔCppA, and ΔPiaA were grown in CY until midlog. Each well of a 96 well high binding ELISA plate (NUNC #430341) was coated with 10⁶ CFU in carbonate-bicarbonate buffer reconstituted from tablet (Sigma C3041). Bacteria were pelleted to bottom of plate by centrifugation and supernatant removed. Plates were air dried overnight. Plates were blocked in 10% heat inactivated fetal bovine serum (FBS) in PBS for two hours. Serum from vaccinated mice was serially diluted 1:2 starting with a 1:50 dilution in 10% FBS in PBS and added to the wells and incubated for one hour at room temperature. For normalization between strains, polyclonal rabbit sera against LytA (gifted by Elaine Tuomanen, St Jude Children's Research Hospital) was initially diluted 1:300 and subsequently diluted 1:2 in 10% FBS. Plates were washed 5× with tris buffered saline (TBS). Secondary antibody (Southern Biotech #1030-04—anti-mouse & 4030-04—anti-rabbit) was diluted 1:2000 in blocking buffer and incubated 1 hour at room temp. Plates were washed 5 times with TBS. Substrate (Sigma #P7998) was added for 30 minutes and OD₄₀₅ read in 96 well plate reader. Normalization of differential coating by wild type and mutant strains was accomplished by dividing all intensities for each strain by the ratio of the intensity of anti-LytA in wild type versus the mutant strain. Purified protein ELISA was performed as above, except wells were coated with 1200 ng purified protein per well overnight at 4° C. in coating buffer.

### Quantification and Statistical Analysis

Unless otherwise specified, all statistics were calculated with Graphpad Prism 6. Mantel-Cox log rank test was used for transmission experiments. Mann-Whitney was used to compare bacterial burdens and viability.

Processing of TnSeq Data and Bottleneck Calculations

The Illumina sequencing read quality was assessed by FastQC (https://www.bioinformatics.babraham.ac.uk/projects/fastqc/). To remove potential phage phiX contamination, the reads were aligned against the phage phiX genome by Bowtie2 (Langmead and Salzberg (2012) Nat Methods 9:357-359) and the unmapped reads were kept for downstream analyses. The adapters and transposon sequences were removed by Trimmomatic (Bolger et al. (2014) Bioinformatics 30:2114-2120). The cleaned reads were demultiplexed by FastX-Toolkit (http://hannonlab.cshl.edu/fastx toolkit/index.html). The reads for each sample were subsequently aligned against the Streptococcus pneumoniae BHN genome sequence by Bowtie2 (parameters: -1 -p 1 -S -n 0 -e 70 -128 -nomaground -y -k 1 -a -m 1 -best).

Each sample from each animal was sequenced individually. Then all the samples from each timepoint in a single animal were combined. This gave us the number and location of unique inserts in each animal.

Bottleneck calculations were determined by dividing the number of unique inserts shared by donor and contact to the number of inserts present in the donor and multiplying by 100 to get a percent. This calculation was done for each cage.

The number of insertions per gene were enumerated and compared between groups by custom R scripts (available at https://github.com/jiangweiyao/FerretTransmission). That data was then used to build the biostatistics model described below to identify transmission factors.

Details of Biostatistics Model

Nine cages were analyzed, with a total of 9 donors and 21 contacts. For each animal, the abundance of each mutant strain was quantified by counting the number of corresponding reads obtained by next-generation sequencing. For donor animals, the infection burden for each strain was defined as log₁₀ (read count+1). For all animals, the read counts were dichotomized to indicate whether the animal was infected (read count >10) or not infected (read count <10) by each strain. Descriptive statistics of infection status of donors and contacts were computed. Also, descriptive statistics of transmission from donor to contact animals were computed in terms of the number of each donor's contacts that became infected. For each donor, the observed transmission rate was computed as the proportion of its contacts that became infected. A final estimate of the probability of transmission was computed as the average of the transmission rates across infected donors. The analyses were performed using version 1.1.13 of the lme4 package for R software (Windows version 3.3.3; www.r-project.org) with script available at https://github.com/jiangweiyao/FerretTransmission.

### Data and Software Availability

BHN97 genome available at https://www.ncbi.nlm.nih.gov/bioproject/420094. Raw TnSeq output available at: https://www.ncbi.nlm.nih.gov/bioproject/497898. R scripts for analysis available at: https://github.com/jiangweiyao/FerretTransmission.

### Primers

Primers used in these studies are provided in Table 3.

TABLE 3

| SEQ | | |
|---|---|---|
| ID | Primer | |
| NO: | Name | Primer Sequence |
| 206 | BHN97 | GGAGCTACTAGATCATGAACTC |
| | ComD | |
| | UP | |
| | FWD | |
| 207 | BHN97 | GAGTCGCTTTTGTAAATTTGGAAGTTTTAATTTT |
| | ComD | AGAAGATGTTATTGAACATC |
| | UP | |
| | REV | |
| 208 | BHN97 | GTTTGCTTCTAAGTCTTATTTCCTCTGCTACTCC |
| | ComD | TTTATCTTCTTCTAAAG |
| | DOWN | |
| | FWD | |
| 209 | BHN97 | GATGTTTCATATTTGCCTCCATATG |
| | ComD | |
| | DOWN | |
| | REV | |
| 210 | BHN97 | GCAAGGTATCAATTAGAAAATAGGC |
| | PiaA | |
| | UP | |
| | FWD | |
| 211 | BHN97 | GTTTGCTTCTAAGTCTTATTTCCAAAAACTCCTT |
| | PiaA | AAACATATTTCAAGTC |
| | UP | |
| | REV | |
| 212 | BHN97 | GAGTCGCTTTTGTAAATTTGGTTAGGAAATGCAT |
| | PiaA | GCAAAAATGCG |
| | DOWN | |
| | FWD | |
| 213 | BHN97 | GCTCCAGGAACAGTTTCA C |
| | PiaA | |
| | DOWN | |
| | REV | |
| 214 | BHN97 | GACCAATCAGAGCAAAAATAAGAAGAG |
| | CppA | |
| | UP | |
| | FWD | |
| 215 | BHN97 | GAG TCG CTT TTG TAA ATT TGG ATG |
| | CppA | AAG TGG ACC AAG ATT ATT AAA AAA |
| | UP | ATA G |
| | REV | |
| 216 | BHN97 | GTTTGCTTCTAAGTCTTATTTCCATTCATAAATT |
| | CppA | CCTCCGTCACTTTTTATTTTAAAG |
| | DOWN | |
| | FWD | |
| 217 | BHN97 | GGTGATTTTGTCAAGGAAGGTACG |
| | CppA | |
| | DOWN | |
| | REV | |
| 218 | BHN97 | CGGATTATTCCTACTTTAAAAGC |
| | CppA | |
| | int | |
| | FWD | |
| 219 | BHN97 | CCA CAA TTC AAC ATT ATT TCT ATC |
| | CppA | |
| | int | |
| | REV | |
| 220 | BHN97 | GCGAGCGAGTGAAGCTGG |
| | SpxB | |
| | UP | |
| | FWD | |
| 221 | BHN97 | GTTTGCTTCTAAGTCTTATTTCC-TTACTATTGA |
| | SpxB | GAGGAAGTTAAAAAAATTTGAACC |
| | UP | |
| | REV | |
| 222 | BHN97 | GAGTCGCTTTTGTAAATTTGG-TTCCTCTCGCCG |
| | SpxB | AAAATCAAATATGAAAC |
| | DOWN | |
| | FWD | |
| 223 | BHN97 | CCATAGTCACTATATACGAGAATTTCGC |
| | SpxB | |
| | DOWN | |
| | REV | |
| 224 | BHN97 | CGGTTAGTGTGGGAGCAACG |
| | SpxR | |
| | UP | |
| | FWD | |
| 225 | BHN97 | GTTTGCTTCTAAGTCTTATTTCCTTAGAATTTCC |
| | SpxR | TGATGCTTACTCATTGAAC |
| | UP | |
| | REV | |
| 226 | BHN97 | GAGTCGCTTTTGTAAATTTGGAAACTAGGAGAAA |
| | SpxR | AGATGATAACATTAAAATCAG |
| | DOWN | |
| | FWD | |
| 227 | BHN97 | GAATCTCTACAGGAACAATAGACTGC |
| | SpxR | |
| | DOWN | |
| | REV | |
| 228 | BHN97 | GCAGAATCTCCCAAGGAAAG |
| | insert | |
| | up | |
| | FWD | |
| 229 | BHN97 | GAAAACAATAAACCCTTGCATATGGTGATAGGAT |
| | insert | AGGAAGCATC |
| | up | |
| | REV- | |
| | SPEC | |
| 230 | BHN97 | CACCTTTTTCATCACCTGTC |
| | insert | |
| | amiF | |
| | FWD | |
| 231 | BHN97 | TTTCCCTTGAACTAGTCGAAG |
| | insert | |
| | amiF | |
| | REV | |
| 232 | PiaA | GTATTCACGAACGAAAATCGATTTATTTCGCATT |
| | comp | TTTGCATGCATTTC |
| | fwd | |
| | Spec | |
| 233 | PiaA | GACAGGTGATGAAAAAGGTGAAAACTAGATCCTT |
| | comp | TTTTGAAAAAATTATATTA |
| | rev | |
| | amiF | |
| 234 | ComD | GTATTCACGAACGAAAATCGATTCATTCAAATTT |
| | comp | CCTCTTAAATCTAATG |
| | fwd | |
| | Spec | |
| 235 | ComD | GACAGGTGATGAAAAAGGTGGAAGGAAAAAGACT |
| | comp | TGCAAAATATTC |
| | rev | |
| | amiF | |
| 236 | SpxB | GTATTCACGAACGAAAATCGATTTATTTAATTGC |
| | comp | GCGTGATTGC |
| | fwd | |
| | Spec | |
| 237 | SpxB | GACAGGTGATGAAAAAGGTGGCATAAATATGATA |
| | comp | CAGTGGAATAG |
| | rev | |
| | amiF | |
| 238 | 5′ | NNNNNGAATTCGGAGGAATTTATGAATGTAAATC |
| | BHN97 | AGATTG |
| | CppA | |
| | EcoRI | |
| 239 | 3′ | NNNNNCTGCAGTCATACTTCTTCAAACCACAATT |
| | BHN97 | CAAC |
| | CppA | |
| | PstI | |
| 240 | Erm F | GGAAATAAGACTTAGAAGCAAAC |
| 241 | Erm R | CCAAATTTACAAAAGCGACTC |
| 242 | Spec F | ATCGATTTTCGTTCGTGAATAC |
| 243 | Spec F | CATATGCAAGGGTTTATTGTTTTC |

We claim:

1. A vaccine composition comprising at least one immunogenic polypeptide comprising at least one Streptococcus pneumoniae (S. pneumoniae) protein having an amino acid sequence set forth as any one of SEQ ID NOs: 1-205 or an immunogenic fragment or variant of any thereof, and a non-naturally occurring pharmaceutically acceptable carrier.

2. The vaccine composition of claim 1, wherein said S. pneumoniae protein is naturally expressed on the surface of S. pneumoniae.

3. The vaccine composition of claim 2, wherein said immunogenic polypeptide lacks a transmembrane domain.

4. The vaccine composition of claim 1, wherein said S. pneumoniae protein is naturally expressed on the surface of S. pneumoniae when S. pneumoniae is undergoing autolysis.

5. The vaccine composition of any one of claims 1-4, wherein said S. pneumoniae protein is conserved among two or more sequenced strains of S. pneumoniae.

6. The vaccine composition of claim 1, wherein said S. pneumoniae protein comprises at least one choline binding protein or an immunogenic fragment or variant thereof.

7. The vaccine composition of claim 6, wherein said S. pneumoniae protein comprises at least one choline binding protein having an amino acid sequence selected from the group consisting of SEQ ID NO: 27, 39, and 82; or an immunogenic fragment or variant of any thereof.

8. The vaccine composition of claim 1, wherein said S. pneumoniae protein comprises at least one protein selected from the group consisting of the sensor kinase of the competence cascade (ComD), the homolog of putative C3-degrading protease (CppA), and the iron transporter PiaA, or an immunogenic fragment or variant of any thereof.

9. The vaccine composition of claim 8, wherein said S. pneumoniae protein comprises at least one protein having an amino acid sequence selected from the group consisting of SEQ ID NOs: 10, 44, and 92, or an immunogenic fragment or variant of any thereof.

10. The vaccine composition of claim 8 or 9, wherein said S. pneumoniae protein comprises at least one of CppA and PiaA.

11. The vaccine composition of any one of claims 1-10, wherein said immunogenic polypeptide further comprises an additional pneumococcal immunogen.

12. The vaccine composition of any one of claims 1-10, further comprising an additional pneumococcal immunogen.

13. The vaccine composition of any one of claims 1-12, further comprising an immunological adjuvant.

14. The vaccine composition of any one of claims 1-13, wherein said composition is formulated for intranasal administration.

15. A method for reducing the mammalian transmission of Streptococcus pneumoniae (S. pneumoniae) by administering to a mammalian subject infected with S. pneumoniae or at risk of infection by S. pneumoniae a vaccine composition of any one of claims 1-14.

16. The method of claim 15, wherein said vaccine composition is administered to said mammalian subject intranasally.

17. The method of claim 15 or 16, wherein said method reduces the transmission of S. pneumoniae from a mother to its offspring.

18. A method for reducing the incidence rate of at least one invasive disease caused by Streptococcus pneumoniae (S. pneumoniae) in a mammalian population by administering to at least one mammalian subject within said mammalian population a vaccine composition of any one of claims 1-14.

19. The method of claim 18, wherein said vaccine composition is administered to said mammalian subject intranasally.

20. The method of claim 18 or 19, wherein said method reduces the transmission of S. pneumoniae from a mother to its offspring.

21. The method of any one of claims 18-20, wherein said at least one invasive disease is selected from the group consisting of pneumonia, acute otitis media, sepsis, meningitis, and bacteremia.

22. A method for identifying genetic factors involved in mammalian transmission of Streptococcus pneumoniae (S. pneumoniae), wherein said method comprises infecting an influenza co-infected ferret with a ferret-transmissible strain of S. pneumoniae comprising a gene mutant library, and analyzing members of said gene mutant library that are able to colonize said infected ferret but not able to transmit or had a reduced transmission rate to contact ferrets to identify genetic factors involved in mammalian transmission.

23. The method of claim 22, wherein said gene mutant library comprises a transposon sequencing (Tn-seq) library.

24. The method of claim 22 or 23, wherein said ferret-transmissable strain of S. pneumoniae is administered to said ferret intranasally.

25. The method of any one of claims 22-24, wherein said ferret-transmissible strain of S. pneumoniae comprises serotype 19F strain BHN97.

26. The method of any one of claims 22-25, wherein said influenza co-infected ferret is co-infected with Influenza/A/5/97 (H3N2).

27. The method of any one of claims 22-26, wherein said influenza co-infected ferret is co-infected intranasally with influenza three days prior to infection with said ferret-transmissible strain of S. pneumoniae.

28. The method of any one of claims 22-27, wherein said method further comprises deletion or mutation of said identified genetic factor in a murine-transmissible strain of S. pneumoniae, infection of a mouse with said murine-transmissible strain of S. pneumoniae, and analyzing transmissibility of said murine-transmissible S. pneumoniae to contact mice.

29. A method for reducing the mammalian transmissibility of Streptococcus pneumoniae by reducing the levels or activity of a protein having an amino acid selected from SEQ ID NOs: 1-205.

30. A method for reducing the mammalian transmissibility of Streptococcus pneumoniae by increasing the levels or activity of a protein that decreases tolerance of desiccation stress.

31. The method of claim 30, wherein said protein comprises a spxB protein or a spxR protein.

32. The method of claim 31, wherein said spxB protein comprises the amino acid sequence set forth as SEQ ID NO: 228 or said spxR protein comprises the amino acid sequence set forth as SEQ ID NO: 229.

33. The vaccine composition of any one of claims 1-14 for use as a medicament.

34. The vaccine composition for use according to claim 33, wherein said medicament is used to reduce the transmission of Streptococcus pneumoniae.

35. The vaccine composition of any one of claims 1-14 for use in reducing the transmission of Streptococcus pneumoniae.
